(19) United States
(12) Patent Application Publication
(10) Pub. No.: US 2026/0174831 A1
(43) Pub. Date: Jun. 25, 2026

(54) METHOD OF TREATMENT OF SOFT TISSUE SARCOMA WITH A PEPTIDE COMPRISING A PCNA INTERACTING MOTIF

(71) Applicant: APIM THERAPEUTICS AS, RISSA (NO)

(72) Inventors: Konstantinos ALEVIZOPOULOS, Yverdon-les-Bains (CH); Marit OTTERLEI, Trondheim (NO)

(21) Appl. No.: 19/463,697

(22) Filed: Jan. 29, 2026

Related U.S. Application Data
(63) Continuation of application No. 17/922,882, now Pat. No. 12,558,400.

(30) Foreign Application Priority Data
May 6, 2020 (GB) 2006699.9

(51) Int. Cl. A61K 38/16; A61P 35/00

(52) U.S. Cl. CPC A61K 38/16; A61P 35/00

(57) ABSTRACT

The present invention relates to pharmaceutical compositions and methods for the treatment of carcinomas and sarcomas. In particular, the invention provides a pharmaceutical composition comprising a peptide or pharmaceutically acceptable salt thereof for use in treating a carcinoma or a sarcoma in a human subject, wherein the peptide comprises an amino acid sequence as set forth in SEQ ID NO: 1 and a cell penetrating peptide and wherein the pharmaceutical composition is systemically administered to the subject weekly to provide a dose of the peptide of about 15-65 mg/m² body surface area (BSA) per week, calculated as the free form of the peptide.

### CROSS REFERENCE TO RELATED APPLICATIONS

This application is a continuation of U.S. application Ser. No. 17/922,882, filed on Nov. 2, 2022, which is a US National stage under § 371 of PCT/EP2021/060992, filed on Apr. 27, 2021, which claims the benefit of priority to GB Patent Application No. 2006699.9, filed on May 6, 2020, which are expressly incorporated herein by reference in their entireties.

### REFERENCE TO SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on Feb. 16, 2026, is entitled “10414-092US2-ST26.xml”, and is 1,048,576 bytes in size.

### FIELD OF THE INVENTION

The present invention relates to pharmaceutical compositions and methods for the treatment of carcinomas and sarcomas. More specifically, the present invention relates to a dosage regimen for a pharmaceutical composition containing a peptide comprising an amino acid sequence as set forth in SEQ ID NO: 1 for use in the treatment of a carcinoma or a sarcoma.

### BACKGROUND TO THE INVENTION

There are more than 100 forms of cancer that can be classified according to the specific cell types from which they originate. The National Cancer Institute (NCI) lists the main types of cancers (https://www.cancer.gov/types), each of which can be further grouped and classified based on the organs or tissues of origin, expression of molecular markers, gene expression profiles, mutational burden, transforming oncogenic mutations and their stage of development.

While different forms and stages of a cancer often have different treatment protocols, some therapeutic molecules have found utility in the treatment of a range of cancers due to their general activity with respect to rapidly proliferating cells.

APIM-peptides are a group of peptides that interact with PCNA (proliferating cell nuclear antigen) via a novel PCNA interacting motif (Gilljam et al., 2009. Identification of a novel, widespread, and functionally important PCNA-binding motif, J. Cell Biol. 186(5), pp. 645-654). The motif has been termed APIM (AlkB homologue 2 (hABH2) PCNA-interacting motif) since it was first identified as mediating the interaction between hABH2 and PCNA, but APIM sequences have now been identified in a wide range of proteins. The PCNA binding motif found in APIM peptides typically is defined using the consensus sequence, [R/K]-[F/W/Y]-[L/I/V/A]-[L/I/V/A]-[K/R] (SEQ ID NO: 2), and it has been determined that a more diverse motif, [R/K/H]-[W/F/Y]-[L/I/V/A/M/S/T/N/Q/C]-[L/I/V/A/M/G/S/T/N/Q/R/H/K/C]-[K/R/H/P] (SEQ ID NO: 3), is present in various proteins that interact with PCNA (see WO2015/067713 incorporated herein by reference). Moreover, an “extended” motif comprising an additional amino acid has also been identified, [R/K/H]-[W/F/Y]-[W/F/Y/L/I/V/M]-[L/I/V/A/M/S/T/N/Q/C]-[L/I/V/A/M/G/S/T/N/Q/R/H/K/C/P]-[K/R/H/P/L/I/V/A/M/G/S/T/N/Q/C] (SEQ ID NO: 4) (see WO2016/177899 incorporated herein by reference).

PCNA is a member of the sliding clamp family of proteins, which is known to be involved in both DNA replication and DNA repair. An essential function of PCNA is to provide replicative polymerases with the high processivity needed for duplication of the genome.

APIM-peptides have been shown to be useful in therapy. Specifically, APIM-peptides have been shown to be effective in sensitizing cells to cytotoxic and cytostatic agents, particularly DNA-damaging agents (WO 2009/104001), microtubule targeting drugs (Søgaard et al., 2018, Oncogene, Vol. 9(14), pp. 11752-11766) and kinase inhibitors (Søgaard et al., 2019, Oncogene, Vol. 10(68), pp. 7185-7197). Thus, APIM peptides have been shown to be useful in combination with other therapeutic agents, such as cytotoxic and/or cytostatic agents, in the treatment of disorders and conditions where it is desirable to inhibit the growth of cells, and in treatments that involve cytostatic therapy, i.e. to prevent or inhibit the unwanted proliferation of cells, e.g. to treat cancer.

While APIM peptides have been shown in some studies to demonstrate activity as apoptosis-inducing cytotoxic agents in their own right, these peptides do not induce apoptosis in healthy cells. For instance, APIM peptides modulate cytokine production in monocytes without inducing apoptosis (e.g. Müller et al., 2013., PLOS One, 8(7), e70430, pp. 1-12 and Olaisen et al., 2015, Cell Signal., Vol. 27(7), pp. 1478-1487). Some animal studies indicate that APIM-peptides have no utility in cancer therapies as a single active agent, e.g. Søgaard et al. (Oncotarget, 2018, Vol. 9(65), pp. 32448-32465) who found that the administration of APIM-peptides alone was ineffective in a muscle-invasive bladder cancer model.

### SUMMARY OF THE INVENTION

In work leading up to the present invention, the inventors have surprisingly determined that APIM-peptides alone are particularly effective in treating carcinomas and sarcomas in human patients. Moreover, the inventors have unexpectedly determined that APIM-peptides are effective at significantly lower concentrations than the concentrations used in animal studies to potentiate the effects of cytotoxic agents.

The therapeutic efficacy of APIM-peptides in humans was particularly surprising because it has been found that the peptides typically cannot be detected in blood within 10-960 minutes after administration. Whilst not wishing to be bound by theory, it is hypothesised that the peptides can enter cells and persist intracellularly for days to provide a long-term action.

Accordingly in one aspect, the invention provides a pharmaceutical composition comprising a peptide or pharmaceutically acceptable salt thereof for use in treating a carcinoma or a sarcoma in a human subject, wherein the peptide comprises an amino acid sequence as set forth in SEQ ID NO: 1 and a cell penetrating peptide and wherein the pharmaceutical composition is systemically administered to the subject weekly to provide a dose of the peptide of about 15-65 mg/m² (e.g. about 15-50 mg/m²) body surface area (BSA) per week, calculated as the free form of the peptide.

Alternatively viewed, the invention provides a method of treating a carcinoma or a sarcoma in a human subject in need thereof, the method comprising administering a pharmaceutical composition comprising a peptide or a pharmaceutically acceptable salt thereof to the subject, wherein the peptide comprises an amino acid sequence as set forth in SEQ ID NO: 1 and a cell penetrating peptide and wherein the pharmaceutical composition is systemically administered to the subject weekly to provide a dose of the peptide of about 15-65 mg/m² (e.g. about 15-50 mg/m²) BSA per week, calculated as the free form of the peptide.

In yet another aspect, the invention provides the use of a peptide or pharmaceutically acceptable salt thereof in the manufacture of a pharmaceutical composition (medicament) for treating a carcinoma or a sarcoma in a human subject, wherein the peptide comprises an amino acid sequence as set forth in SEQ ID NO: 1 and a cell penetrating peptide and wherein the pharmaceutical composition is systemically administered to the subject weekly to provide a dose of the peptide of about 15-65 mg/m² (e.g. about 15-50 mg/m²) body surface area (BSA) per week, calculated as the free form of the peptide.

### DETAILED DESCRIPTION

The term “carcinoma” refers to types of cancer that develop from epithelial cells, including cancers derived from the lining of internal organs. Carcinomas may be sub-categorised based on their histology.

For instance, carcinomas comprising glandular-related tissue cytology, tissue architecture, and/or gland-related molecular products, e.g., mucin, are categorized as adenocarcinomas.

Squamous cell carcinomas include carcinomas have features indicative of squamous cell differentiation (intercellular bridges, keratinization, squamous pearls).

Adenosquamous carcinomas are mixed tumors containing both an adenocarcinoma and squamous cell carcinoma, typically where each of these cell types comprise at least 10% of the tumor volume.

Anaplastic or undifferentiated carcinomas are a heterogeneous group of carcinomas that feature cells lacking distinct histological or cytological evidence of any of the more specifically differentiated neoplasms.

Large cell carcinomas are composed of large, monotonous rounded or overtly polygonal-shaped cells with abundant cytoplasm.

Small cell carcinomas contain cells that are usually round and less than approximately 3 times the diameter of a resting lymphocyte, and with little evident cytoplasm. Occasionally, small cell malignancies may themselves have significant components of slightly polygonal and/or spindle-shaped cells.

Thus, in some embodiments, the carcinoma to be treated according to the invention is an adenocarcinoma, squamous cell carcinoma, adenosquamous carcinoma, anaplastic carcinoma, large cell carcinoma or small cell carcinoma.

In some embodiments, the carcinoma is a carcinoma of the lung, pancreas, cervix, urethra or ovaries, e.g. an adenocarcinoma or squamous cell carcinoma.

Thus, in some embodiments, the adenocarcinoma is a pancreatic or lung adenocarcinoma.

In some embodiments, the squamous cell carcinoma is a squamous cell carcinoma of the cervix or urethra.

In some embodiments, the carcinoma of the lung is non-small cell lung cancer. In some embodiments, the non-small cell lung cancer is a large cell carcinoma or an adenocarcinoma.

In some embodiments, the carcinoma of the ovaries is an ovarian granulosa cell tumor. In some embodiments, the carcinoma of the ovaries includes epithelial carcinomas of the ovaries, fallopian tubes or primary peritoneal cancers. Thus, in some embodiments, the carcinoma is an epithelial carcinoma of the ovaries, an epithelial carcinoma of the fallopian tubes or a primary peritoneal cancer.

The term “sarcoma” refers to types of cancer that develop from mesenchymal cells, which form connective tissue, i.e. bone, cartilage, fat, vascular, or hematopoietic tissues. Sarcomas typically are classified based on the specific tissue and type of cell from which the tumor originates and may be generally categorized as bone or soft tissue sarcomas.

In some embodiments, the sarcoma to be treated according to the invention is a soft tissue sarcoma. In some embodiments, the sarcoma is a myosarcoma (e.g. rhabdomyosarcoma or leiomyosarcoma), liposarcoma, undifferentiated pleomorphic sarcoma or synovial sarcoma.

In some embodiments, the myosarcoma is a leiomyosarcoma, such as a uterine leiomyosarcoma.

In some embodiments, the undifferentiated pleomorphic sarcoma is a metastatic undifferentiated pleomorphic sarcoma.

The terms “treating” or “treatment” as used herein refer broadly to any effect or step (or intervention) beneficial in the management of a clinical condition or disorder. Treatment therefore may refer to reducing, alleviating, ameliorating, slowing the development of, or eliminating one or more symptoms of the carcinoma or sarcoma that is being treated, relative to the symptoms prior to treatment, or in any way improving the clinical status of the subject. A treatment may include any clinical step or intervention which contributes to, or is a part of, a treatment programme or regimen. In particular, said treatment may comprise reduction in the size or volume of the carcinoma or sarcoma being treated.

A treatment may include delaying, limiting, reducing or preventing the onset of one or more symptoms of the carcinoma or sarcoma, for example relative to the carcinoma or sarcoma or symptom prior to the treatment. Thus, treatment explicitly includes both absolute prevention of occurrence or development of symptom of the carcinoma or sarcoma, and any delay in the development of the carcinoma or sarcoma or symptom thereof, or reduction or limitation on the development or progression of the carcinoma or sarcoma or symptom thereof.

Treatment according to the invention thus includes killing, inhibiting or slowing the growth of carcinoma or sarcoma cells, or the increase in size of a body or population of carcinoma or sarcoma cells (e.g. in a tissue, tumor or growth), reducing carcinoma or sarcoma cell number or preventing spread of carcinoma or sarcoma cells (e.g. to another anatomic site), reducing the size of a cell growth etc. The term “treatment” does not necessarily imply cure or complete abolition or elimination of carcinoma or sarcoma cell growth, or a growth of carcinoma or sarcoma cells.

In some embodiments, treatment is measured using the RECIST criteria (Response Evaluation Criteria In Solid Tumors). The RECIST criteria are based on whether tumors shrink, stay the same, or get bigger based on measurements using, for example, x-rays, CT scans, or MRI scans. The types of response a patient can have are a complete response (CR), a partial response (PR), progressive disease (PD), and stable disease (SD). Thus, treatment refers to a patient showing a complete response (CR), a partial response (PR) or stable disease (SD). In some embodiments, treatment refers to a patient showing SD. In some embodiments, treatment refers to a patient showing SD for at least 3 months, e.g. 4, 5, 6 or more months, as measured from the start of treatment.

A “complete response” refers to disappearance of all target lesions.

A “partial response” refers to at least a 30% decrease in the sum of the lesion diameter (LD) of target lesions, taking as reference the baseline sum LD.

“Stable disease” refers to situations where there is neither sufficient shrinkage to qualify as a PR nor sufficient increase to qualify as PD, taking as reference the smallest sum LD since the treatment started.

“Progressive disease” refers to situations where there is at least a 20% increase in the sum of the LD of target lesions, taking as reference the smallest sum LD recorded since the treatment started or the appearance of one or more new lesions

The terms “subject” and “patient” herein refer to a human, i.e. a human having a carcinoma or sarcoma as defined herein in need to treatment.

The peptide for use in the invention contains an amino acid sequence as set forth in SEQ ID NO: 1, which may be viewed as a “PCNA interacting motif”.

The term “PCNA interacting motif” refers to a sequence of consecutive amino acids within a peptide that functions to facilitate the interaction of the peptide with PCNA. Thus, the peptide of the invention may be characterised insofar as it must be capable of interacting with a PCNA protein. In other words, the peptide for use in the invention must be a competent and/or proficient PCNA interacting molecule. The terms “PCNA interacting motif” and “APIM sequence” may be used interchangeably herein.

The PCNA protein used to determine the capacity and/or affinity of the peptide: PCNA interaction may be from any suitable source, e.g. a PCNA from any animal, particularly a mammal such as a human, rodent (e.g. mouse, rat) or any other non-human animal. In preferred embodiments, the peptide: PCNA interaction is determined, characterised or assessed using human PCNA protein. The interaction involves direct binding of the peptide to the PCNA protein.

The peptide is an isolated peptide and most preferably a synthetic peptide. In other words, the peptide is a non-native, i.e. non-naturally occurring, molecule.

In order that the peptide, which is capable of interacting with PCNA, may function in the methods and uses of the invention, the peptide must be capable of entering the target cells, i.e. crossing the cell membrane into the cytosol (cytoplasm), and optionally into one or more other cellular locations, e.g. the nucleus. As noted above, it is hypothesised that the ability of the peptide to persist intracellularly for days may be responsible for its long-term action.

Thus, the peptide comprises a domain that assists the transit of the peptide across the cell membrane, i.e. the peptide is provided as a fusion peptide or chimeric peptide (a peptide formed from two or more domains that are not normally found together in nature). In particular, a peptide for use in the invention comprises a cell penetrating peptide (CPP), which may alternatively be termed an uptake or import peptide, or a peptide transduction domain.

The final size of the peptide will be dependent on the size and number of the domains that make up said peptide, i.e. the PCNA interacting motif and CPP may be viewed as domains of the peptide. Thus, a domain may be viewed as a distinct portion (i.e. a sequence within the full-length amino acid sequence) of the peptide that can be assigned or ascribed a particular function or property.

The peptide comprises at least two domains, i.e. the PCNA interacting motif domain (SEQ ID NO: 1) and the CPP. However, the peptide may comprise additional domains that may facilitate its function and/or stability, e.g. the capacity of the peptide to interact with its target, PCNA. Thus, the peptide may comprise 2, 3, 4 or 5 domains, e.g. 6, 7, 8, 9, 10, 12, 15 or more domains. For example, in some embodiments the peptide may comprise one or more linker domains, i.e. a domain that interspaces between two other domains, i.e. occupies the space in between and connects two domains of the peptide.

In some embodiments, the linker domain may be inert, i.e. it may have no physiological function in the target cell in which the peptide is active and simply functions to physically separate the other domains in the peptide. However, in some embodiments, the linker domain may have an additional function. For instance, the linker domain may also function as a cleavage domain, i.e. the linker domain may contain a peptide bond that is susceptible to cleavage under physiological conditions, e.g. inside the target cell, such that the peptide is cleaved following its uptake.

In some embodiments, the peptide may comprise a domain that directs the peptide to a cellular or subcellular location, e.g. a signal peptide (also known as a target or transit peptide), such as a nuclear localization signal (NLS) sequence. Thus, in some embodiments, the one or more linker domains function as a signal peptide, e.g. an NLS, i.e. the linker may conveniently be a signal peptide, such as an NLS. Alternatively put, a signal peptide domain may function as a linker domain in some embodiments. In some embodiments, the peptide may comprise a signal peptide (e.g. NLS) in addition to one or more linker domains, e.g. inert linker domains.

In an exemplary embodiment, the peptide comprises a PCNA interacting motif domain as set forth in SEQ ID NO: 1, a CPP and a linker domain. In a further exemplary embodiment, the peptide may also comprise a nuclear localisation signal sequence domain. In still another embodiment the nuclear localization signal sequence domain may function as a linker domain.

Thus, it will be seen that in such embodiments the peptide of the invention may take the form of a construct containing (i.e. comprising) a peptide which comprises a PCNA interacting motif as set forth in SEQ ID NO: 1, together with a CPP domain that facilitates its cellular uptake and optionally additional domains. In this aspect the invention may accordingly be seen to provide a construct comprising a peptide which is capable of interacting with PCNA.

Accordingly, the invention may provide a pharmaceutical composition containing a construct for use in the methods and uses of the invention comprising (i) a peptide comprising a PCNA-interacting motif as set forth in SEQ ID NO: 1, and (ii) a cell penetrating peptide.

Cell penetrating peptide (CPP) technology has developed greatly over recent years and a wide variety of cell penetrating peptides are known and described in the art. Indeed a range of such peptides is commercially available. Cell penetrating peptides may vary greatly in size, sequence and charge, and indeed in their mechanism of function (which is presently not known for some peptides and not fully elucidated for others), but share the common ability to translocate across the plasma membrane and deliver an attached or associated moiety (the so-called “cargo”) into the cytoplasm of a cell. CPPs are thus peptide-based delivery vectors.

Whilst CPPs are not characterized by a single structural or functional motif, tools to identify CPPs are available and the skilled person can readily determine whether a peptide sequence may function to facilitate the uptake of the peptide of which it forms a domain, i.e. whether a peptide sequence may function as a CPP. For example, Hansen et al (Predicting cell-penetrating peptides, Advanced Drug Delivery Reviews, 2008, 60, pp. 572-579), provides a review of methods for CPP prediction based on the use of principal component analysis (“z-predictors”) and corresponding algorithms based on original work by Hällbrink et al (Prediction of Cell-Penetrating Peptides, International Journal of Peptide Research and Therapeutics, 2005, 11(4), pp. 249-259). In brief, the methodology works by computing z-scores of a candidate peptide as based on a numerical value and an associate range. If the z-scores fall within the range of known CPP z-scores, the examined peptides are classified as CPPs. The method was shown to have high accuracy (about 95% prediction of known CPPs).

Additional methods for the prediction of CPPs have been developed subsequently (see e.g. Sanders et al., Prediction of Cell Penetrating Peptides by Support Vector Machines, PLOS Computational Biology, 2011, 7(7), pp. 1-12, herein incorporated by reference) and a CPP database is available (Gautam et al., CPPSite: a curated database of cell penetrating peptides, Database, 2012, Article ID bas015 and http://crdd.osdd.net/raghava/cppsite/index.php, both herein incorporated by reference). Accordingly, any suitable CPP may find utility in the invention and, as discussed below, a variety of CPPs have already been identified and tested and could form the basis for determining and identifying new CPPs.

CPPs may be derived from naturally-occurring proteins which are able to translocate across cell membranes such as the Drosophila homeobox protein Antennapedia (a transcriptional factor), viral proteins such as the HIV-1 transcriptional factor TAT and the capsid protein VP22 from HSV-1, and/or they may be synthetically-derived, e.g. from chimeric proteins or synthetic polypeptides such as polyarginine. As noted above, there is not a single mechanism responsible for the transduction effect and hence the design of CPPs may be based on different structures and sequences. Cell penetrating peptides are also reviewed in Jarver et al. 2006 (Biochimica et Biophysica Acta 1758, pages 260-263). U.S. Pat. No. 6,645,501, WO2015/067713 and WO2016/177898 (all herein incorporated by reference) further describe various cell penetrating peptides which might be used.

Antennapedia-derived CPPs (Antp class) represent a class of CPPs based around the 16 amino acid Penetratin sequence, which corresponds to the third loop of antennapedia protein and was shown to be responsible for translocation of the protein. Penetratin has been extensively developed as a delivery vehicle, including particularly for pharmaceutical use, and a wide range of Penetratin derivatives and modified sequences have been proposed and described. Reference may be made in particular to WO 91/1891, WO 00/1417, WO 00/29427, WO 2004/069279 and U.S. Pat. No. 6,080,724 (herein incorporated by reference). Thus, the 16 amino acid sequence of Penetratin may be modified and/or truncated, or the peptide may be chemically-modified or retro-, inverso- or retro-inverso analogues may be made whilst retaining cell-penetrating activity.

Another group of cell penetrating peptides which may be used are based on the HIV-TAT sequence and HIV-TAT and fragments thereof. Various TAT-based CPPs are described in U.S. Pat. No. 5,656,122 (herein incorporated by reference). An exemplary HIV-TAT peptide as used in the Examples below is RKKRRQRRR (SEQ ID NO: 38) but it will readily be appreciated that longer or shorter TAT fragments may be used.

As mentioned above, no particular structural features or sequence motifs are common to all CPPs. However, various classes of CPPs may be identified by particular features, such as for example peptides which are amphipathic and net positively charged. Other groups of CPPs may have a structure exhibiting high α-helical content. Another group may be peptides characterised by a high content of basic amino acids. CPPs may thus be or may comprise oligomers of basic amino acids such as arginine e.g. 5 to 20, 6 to 15 or 6 to 12 R residues e.g. R₇ (SEQ ID NO: 37), R₈ (SEQ ID NO: 39) or R₁₁ (SEQ ID NO: 40) or QSR: (SEQ ID NO: 41). These CPPs represent a preferred group of CPPs for use in the invention.

Thus, in some embodiments, the domain that facilitates the uptake of the oligopeptidic compound (e.g. CPP) may be defined as a peptide of 4-30 amino acids (e.g. 5-29, 6-28, 7-27, 8-26, 9-25 etc. amino acids), wherein at least 4 amino acids, optionally at least 4 consecutive amino acids, (e.g. at least 5, 6, 7, 8, 9, 10 or 11 amino acids, e.g. 4-20, 5-19, 6-18, 7-17, 8-16, 9-15, 10-14, 11-13 amino acids) are positively charged amino acids, preferably selected from K, R or H.

Proline-rich amphipathic peptides are another class of CPP and such peptides characterised by the presence of pyrrolidine rings from prolines are described in Pujals et al. 2008 Advanced Drug Delivery Reviews 60, pages 473-484 (herein incorporated by reference).

Other successfully developed CPPs include pVEC (Elmquist et al. 2003 Biol. Chem 384, pages 387-393; Holm et al. 2005 Febs Lett. 579, pages 5217-5222, all herein incorporated by reference) and calcitonin-derived peptides (Krauss et al. 2004 Bioorg. Med. Chem. Lett., 14, pages 51-54, herein incorporated by reference).

Commercially available CPPs include Chariot, based on the Pep-1 peptide (Active Motif, France), the Syn-B vectors based on the protegrin peptide PG-1 (Syntem, France), and Express-si Delivery based on the MPG peptide from Genospectra, USA.

Other CPPs include the R41, R8, M918 and YTA-4 peptides (SEQ ID NOs: 866-869, respectively) disclosed in Eriksson et al. 2013, Antimicrobial Agents and Chemotherapy, vol. 57(8), pp. 3704-3712 (incorporated herein by reference).

In some embodiments the CPPs may be cyclic peptides, such as those disclosed in Oh et al., 2014, Mol. Pharmaceutics, vol. 11, pp. 3528-3536 (incorporated herein by reference). In particular, the CPPs may be amphiphilic cyclic CPPs, particularly containing tryptophan and arginine residues. In some embodiments the CPPs may be cyclic polyarginine peptides and may be modified by the addition of a fatty acyl moiety, e.g. octanoyl, dodecanoyl, hexadecanoyl, N-acetyl-L-tryptophanyl-12-aminododecanoyl etc. Suitable cyclic CPPs for use in the invention are presented in SEQ ID NOs: 870-876.

In addition to publicly available and reported CPPs, novel or derivative CPP peptides may be designed and synthesized based on known or reported criteria (e.g. known CPP sequences or features such as basic amino acid content, α-helical content etc. as discussed above). Additionally, randomly-designed or other peptides may be screened for CPP activity, for example by coupling or attaching such a peptide containing a reporter molecule, e.g. a detectable label or tag such as a fluorescent tag to the desired cargo (e.g. a peptide comprising SEQ ID NO: 1) and testing to see if the construct is translocated across the cell membrane, for example by adding these peptides to live cells followed by examination of cellular import e.g. using confocal microscopy.

It may in some cases be observed that successful or efficient delivery may be dependent, or may vary depending, on the precise nature of the cargo (e.g. cargo peptide sequence) and/or the CPP used. It would be well within the routine skill of the person skilled in the art to determine optimum peptide sequences and combinations etc, and to test and/or modify cargo and/or CPP sequence or structure etc.

Thus, in some embodiments the CPP is selected from any one of:
(i) an antennapedia class peptide;
(ii) a protegrin class peptide;
(iii) a HIV-TAT class peptide;
(iv) an amphipathic class peptide selected from an amphipathic and net positively charged peptide, a proline-rich amphipathic peptide, a peptide based on the Pep-1 peptide and a peptide based on the MPG peptide;
(v) a peptide exhibiting high α-helical content;
(vi) a peptide comprising oligomers of basic amino acids;
(vii) pVEC;
(viii) a calcitonin-derived peptide and
(ix) an amphiphilic cyclic CPP.

In some embodiments, the CPP is selected from a sequence selected from any one of SEQ ID NOs: 5-876 or a fragment and/or derivative thereof. The details and properties of the CPPs identified in SEQ ID NOs: 43-865 can be found at http://crdd.osdd.net/raghava/cppsite/index.php, CPPSite: A database of cell penetrating peptides (herein incorporated by reference).

In preferred embodiments the CPP comprises an amino acid sequence as set forth in SEQ ID NO: 37, 39 or 40.

In some embodiments, the peptide also comprises one or more domains that provide a signal (target or transit) sequence. In some embodiments, the signal sequence may target the peptide to a specific cell type. Additionally or alternatively, in some embodiments the peptide may comprise a signal peptide that localises the peptide to a specific intracellular compartment, e.g. the nucleus. In some embodiments, the peptide is targeted to the cytosol, which may be achieved without an additional signal peptide, i.e. the CPP, is sufficient to direct or localise the peptide to the cytosol of a cell.

The signal sequence or signal sequence domain may thus be viewed as any sequence which acts to localise, or alternatively put, to direct, translocate or transport, the peptide to any desired location e.g. to any desired cell type or subcellular location, e.g. nucleus.

As mentioned above, the peptide for use in the invention may comprise one or more signal sequences (i.e. one or more domains that function as signal sequences), e.g. a signal peptide which directs the peptide into a particular subcellular compartment, such as the nucleus.

Nuclear localisation signals (NLSs) are again well-known in the art and widely described in the literature. For instance, a searchable database of known and predicted NLSs is available, see e.g. Cokol et al (Finding nuclear localization signals, EMBO Reports, 2000, 1(5), pp. 411-415, herein incorporated by reference). The PSORT II database, http://psort.hgc.jp/(herein incorporated by reference) can be used for the prediction of nuclear localization of proteins based on NLSs. Accordingly, any known or functional NLS may find utility in the invention.

An NLS may vary in length and/or sequence and a wide range of specific NLS sequences have been described. In general, however, it has been found that peptides comprising positively charged amino acids (notably lysine (K), arginine (R) and/or histidine (H)) may function as an NLS. An exemplary NLS may thus be a peptide of e.g. 4-20, more particularly 4-15, 4-12, 4-10 or 4-8 amino acids, wherein at least 4 amino acids (and more particularly at least 60, 70, 75, 80, 85, or 90% of the amino acid residues in the NLS peptide) are positively charged amino acids, preferably selected from K, R or H. Such an exemplary NLS may for example have or comprise the sequence RKRH (SEQ ID NO: 877).

Nuclear localisation signals, including both actual experimentally-determined and predicted or proposed NLS sequences, and strategies for identifying NLSs are also described in Lange et al., J. Biol. Chem. 2007, 282(8), 5101-5105; Makkerh et al., Current Biology 1996, 6(8), 1025-1027; Leslie et al., Methods 2006, 39, 291-308; and Lusk et al. Nature Reviews MCB 2007, 8, 414-420 (all herein incorporated by reference).

A classical NLS consists of either one (monopartite) or two (bipartite) stretches of basic amino acids. A monopartite NLS may be exemplified by the SV40 large T antigen NLS (¹²⁶PKKKRKV¹³² [SEQ ID NO: 878]) and a bipartite NLS by the nucleoplasmin NLS

¹⁵⁵KRPAATKKAGQAKKKK¹⁷⁰ [SEQ ID NO: 879]).

The monopartite NLS consensus sequence K-[K/R]-X-[K/R] (SEQ ID NO: 880) has been proposed and accordingly an NLS according to the present invention may in one embodiment comprise or consist of such a consensus sequence (where X is any amino acid).

A representative bipartite NLS according to the invention may have the sequence KR-[X]₅₋₂₀-KKKK (SEQ ID NO: 881), e.g. KR-X₁₀-KKKK (SEQ ID NO: 882) (where X is any amino acid).

An alternative exemplary bipartite NLS may take the form RKRH-[X]₂₋₁₀-KK (SEQ ID NO: 883) e.g. RKRH-X₂-KK (SEQ ID NO: 884), for example RKRH-II-KK (SEQ ID NO: 885).

The oncoprotein c-myc NLS differs from classical NLSs in that only 3 of 9 amino acid residues are basic (PAAKRVKLD [SEQ ID NO: 886]), indicating that an NLS need not necessarily conform to the consensus or classical sequences given above. Makkerh et al (supra) describe NLS sequences in which a cluster of basic amino acids (e.g. KKKK [SEQ ID NO: 887]) is flanked by neutral and acidic residues, for example PAAKKKKLD (SEQ ID NO: 888).

Other possible NLS sequences which may be given by way of example include: PKKKRKVL (SEQ ID NO: 889), KKKRK (SEQ ID NO: 890), KKKRVK (SEQ ID NO: 891), KKKRKVL (SEQ ID NO: 892) and RKKRKVL (SEQ ID NO: 893). Any NLS which is a derivative of a known NLS e.g. the SV40, nucleoplasmin, UNG2 or c-myc NLS may be used.

A putative, proposed or predicted NLS sequence can be tested for NLS activity using principles and assays known and described in the art. For example, a candidate NLS sequence may be attached to the desired cargo (in this case a peptide as defined herein) and the construct may be provided with a detectable reporter molecule (e.g. a tag or label which may be visualised, for example a fluorescent label) and contacted with a test cell. Distribution of the construct in the cell may then be determined.

Thus, by way of summary, the skilled person will be aware of suitable signal sequences. In a particularly preferred embodiment, the peptide includes an NLS signal sequence from the SV40 protein, which comprising the amino acid sequence KKKRK (SEQ ID NO: 890).

Thus, in some embodiments, the peptide comprises a signal sequence (i.e. a domain comprising a signal peptide) that localizes or directs the peptide to a subcellular location, such as an NLS and may be selected from any one of:
(i) a peptide of 4-20 amino acids, wherein at least 4 amino acids are positively charged amino acids, preferably selected from K, R or H; and/or
(ii) a sequence selected from any one of SEQ ID NOs: 877-893 or a fragment and/or derivative thereof.

In some embodiments the nuclear localisation signal sequence comprises a sequence selected from any one of SEQ ID NOs: 877-893 or a fragment and/or derivative thereof, preferably wherein said fragment and/or derivative comprises at least 4 positively charged amino acids, preferably selected from any of K, R or H.

In some embodiments a peptide or construct according to the present invention may comprise at least three domains, including (i) PCNA interacting motif as set forth in SEQ ID NO: 1 (APIM sequence), (ii) a linker domain, which may in some embodiments comprise a nuclear localisation signal sequence, and (iii) a CPP.

The separate elements or components (domains) of a peptide according to the present invention may be contained or presented in any order, but preferably in the orders indicated above (e.g. APIM sequence-CPP or APIM sequence-linker-CPP).

In some embodiments, the APIM motif is located at or towards the N-terminus of the peptide. For instance, the APIM motif may be described as being N-terminal to the CPP and optionally N-terminal to the linker sequence, if present.

The domains (which may be viewed as components, elements or separate parts) of a peptide of the invention as described herein may be attached or linked to one another in any desired or convenient way according to techniques well known in the art. Thus, the domains may be linked or conjugated chemically, e.g. using known chemical coupling technologies or the compound or constructs may be formed as a single whole using genetic engineering techniques e.g. techniques for forming fusion proteins, or they may simply be synthesized as a whole, e.g. using peptide synthesis techniques. In preferred embodiments, the domains are linked by peptide bonds.

The domains may be linked directly to each other or they may be linked indirectly by means of one or more linker (or spacer) sequences. Thus, a linker sequence may interspace or separate two or more individual domains (i.e. parts, e.g. or separate motif elements) in a peptide. The precise nature of the linker sequence is not critical and it may be of variable length and/or sequence, for example it may have 0-40, more particularly 0-20, 0-15, 0-12, 0-10, 0-8, 0-7, 0-6, 0-5, 0-4 or 0-3 residues e.g. 1, 2 or 3 or more residues. By way of representative example the linker sequence, if present, may have 1-15, 1-12, 1-10, 1-8, 1-7, 1-6, 1-5 or 1-4 residues etc. The nature of the residues is not critical and they may for example be any amino acid, e.g. a neutral amino acid, or an aliphatic amino acid, or alternatively they may be hydrophobic, or polar or charged or structure-forming, e.g. proline. A range of different linker sequences have been shown to be of use, including short (e.g. 1-7) sequences of neutral and/or aliphatic amino acids.

Exemplary linker sequences thus include any single amino acid residue, e.g. A, I, L, V, G, R, Q, T, or W, or a di-, tri-tetra-penta- or hexa-peptide composed of such residues.

As representative linkers may be mentioned I, II, IL, R, W, WW, WWW, RIL, RIW, GAQ, GAW, VAT, IILVI (SEQ ID NO: 894), IILVIII (SEQ ID NO: 895), GILQ (SEQ ID NO: 896), GILQWRK (SEQ ID NO: 897) etc.

As mentioned above, in some embodiments, the linker contains an NLS sequence. Thus, in a particularly preferred embodiment, the linker comprises WKKKRKI (SEQ ID NO: 898).

In a preferred embodiment, the peptide comprises a PCNA interacting motif (APIM sequence) as set forth in SEQ ID NO: 1 and a cell penetrating signal sequence as set forth in SEQ ID NO: 37, 39 or 40. For instance, in some embodiments, the peptide comprises a PCNA interacting motif as set forth in SEQ ID NO: 1, a linker domain as set forth in SEQ ID NO: 890 or 898 and a cell penetrating signal sequence as set forth in SEQ ID NO: 37, 39 or 40, preferably SEQ ID NO: 40. In some particular embodiments, the peptide comprises a PCNA interacting motif as set forth in SEQ ID NO: 1, a linker domain as set forth in SEQ ID NO: 898 and a cell penetrating signal sequence as set forth in SEQ ID NO: 40, e.g. a sequence as set forth in any one of SEQ ID NOs: 914-916 or 918-920, preferably SEQ ID NO: 914 or 918, most preferably SEQ ID NO: 914.

Furthermore, in some embodiments a peptide according to the invention may contain more than one PCNA-interacting motif. A peptide may for example contain 1-10, e.g. 1-6, or 1-4 or 1-3 or one or two motifs. In some embodiments, the motifs may be identical, i.e. the peptide may comprise more than one sequence as set forth in SEQ ID NO: 1. In some embodiments, the motifs may be different, i.e. SEQ ID NO: 1 and one or more other motifs. Suitable alternative motifs are described in the art as described above. Within a peptide also containing a signal sequence, such motifs may be spaced or located according to choice, e.g. they may be grouped together, or they may be separated by other domains, e.g. motif-motif-CPP, motif-linker-motif-CPP; or motif-linker-motif-motif-CPP; or motif-motif-linker-CPP etc.

As referred to herein a “fragment” may comprise at least 30, 40, 50, 60, 70, 80, 85, 90, 95, 96, 97, 98 or 99% of the amino acids of the sequence from which it is derived. Said fragment may be obtained from a central or N-terminal or C-terminal portions of the sequence. Whilst the size of the fragment will depend on the size of the original sequence, in some embodiments the fragments may be 1, 2, 3, 4, 5, 6, 7, 8, 9, 10, 11, 12, 13, 14, 15 or more amino acid residues shorter than the sequence from which it is derived, e.g. 1-10, 2-9, 3-8, 4-7 amino acid residues shorter than the sequence from which it is derived.

As referred to herein a “derivative” of a sequence is at least 55, 60, 65, 70, 75, 80, 85, 90, 95, 96, 97, 98 or 99% identical to the sequence to which it is compared.

Sequence identity may be determined by, e.g. using the SWISS-PROT protein sequence databank using FASTA pep-cmp with a variable pamfactor, and gap creation penalty set at 12.0 and gap extension penalty set at 4.0, and a window of 2 amino acids. Preferably said comparison is made over the full length of the sequence, but may be made over a smaller window of comparison, e.g. less than 200, 100, 50, 20 or 10 contiguous amino acids.

Preferably such sequence identity related peptides, i.e, derivatives, are functionally equivalent to the peptides which are set forth in the recited SEQ ID NOs. Similarly, the peptides with sequences as set forth in the SEQ ID NOs. may be modified without affecting the sequence of the polypeptide as described below.

Furthermore, “fragments” as described herein may be functional equivalents. Preferably these fragments satisfy the identity (relative to a comparable region) conditions mentioned herein.

As referred to herein, to achieve “functional equivalence” the peptide may show some reduced efficacy in performing the function relative to the parent molecule (i.e. the molecule from which it was derived, e.g. by amino acid substitution), but preferably is as efficient or is more efficient. Thus, functional equivalence may relate to a peptide which is effective in localizing or directing the peptide into the cell, e.g. to facilitate to the uptake of the peptide as described above. This may be tested by comparison of the effects of the derivative peptide relative to the peptide from which it is derived in a qualitative or quantitative manner, e.g. by performing the in vitro analyses described above. Where quantitative results are possible, the derivative is at least 30, 50, 70 or 90% as effective as the parent peptide.

Functionally-equivalent peptides which are related to or derived from the parent peptide, may be obtained by modifying the parent amino acid sequence by single or multiple amino acid substitution, addition and/or deletion (providing they satisfy the above-mentioned sequence identity requirements), but without destroying the molecule's function. Preferably the parent sequence has less than substitutions, additions or deletions, e.g. less than 10, 5, 4, 3 or 2 such modifications. Such peptides may be encoded by “functionally-equivalent nucleic acid molecules” which may be generated by appropriate substitution, addition and/or deletion of one or more bases.

Representative peptides containing a PCNA interacting motif as set forth in SEQ ID NO: 1 include:

(SEQ ID NO: 899)
MDRWLVKRILVATK,
(SEQ ID NO: 900)
MDRWLVKRILKKKRKVATKG,
(SEQ ID NO: 901)
MDRWLVKGAQPKKKRKVLRQIKIWFQNRRMKWKK,
(SEQ ID NO: 902)
MDRWLVKGAWKKKRVKIIRKKRRQRRRK,
(SEQ ID NO: 903)
MDRWLVKGAWKKKRKIIRKKRRQRRRG,
(SEQ ID NO: 904)
MDRWLVKGAWKKKRKIIRKKRRQRRRK,
(SEQ ID NO: 905)
MDRWLVKRIWKKKRKIIRKKRRQRRRK,
(SEQ ID NO: 906)
MDRWLVKWWWKKKRKIIRKKRRQRRRK,
(SEQ ID NO: 907)
MDRWLVKWWRKRHIIKKRKKRRQRRRK,
(SEQ ID NO: 908)
MDRWLVKRIWKKKRKIIRRRRRRRRRRRK,
(SEQ ID NO: 909)
MDRWLVKRIWKKKRKIIRQIKIWFQNRRMKWKK,
(SEQ ID NO: 910)
MDRWLVKWKKKRKIRRRRRRRRRRRK,
(SEQ ID NO: 911)
MDRWLVKWKKKRKIRKKRRQRRRK,
(SEQ ID NO: 912)
MDRWLVKWRKRHIRKKRRQRRRK,
(SEQ ID NO: 913)
MDRWLVKGAWRKRHIRKKRRQRRRK,
(SEQ ID NO: 914)
MDRWLVKWKKKRKIRRRRRRRRRRR,
(SEQ ID NO: 915)
MDRWLVKKKKRKRRRRRRRRRRRK,
(SEQ ID NO: 916)
MDRWLVKKKKRKRRRRRRRRRRR,
(SEQ ID NO: 917)
MDRWLVKRIWKKKRKIIRWLVKWWWRKKRRQRRRK

The peptides shown above comprise N-terminal amino acids that do not form part of the domains that are essential for the peptides to have activity in the methods and uses of the invention, i.e. an “MD” sequence. Some of the peptides may also comprise N-terminal modification, e.g. acetyl groups. These additional amino acids and modifications may facilitate the production of the peptides, e.g. in vitro or in vivo, and/or help to protect the peptides from degradation in vivo. It will be evident that the peptides do not require these additional amino acids or modifications for their activity. Accordingly, further representative sequences according to the invention include any of SEQ ID NOs: 899 to 917, omitting the N-terminal “MD”, e.g. the peptide may comprise the amino acid sequence RWLVKWKKKRKIRRRRRRRRRRR, RWLVKKKKRKRRRRRRRRRRRK or RWLVKKKKRKRRRRRRRRRRR (SEQ ID NOs: 918-920). Furthermore, the presence of additional amino acids or modifications at either terminus would not be expected to disrupt or inhibit the function of the peptides described herein. Thus, in some embodiments, the peptide may comprise an N-terminal sequence, e.g. a sequence at the N-terminus that does not comprise a domain defined above, e.g. a so-called N-terminal flanking sequence. In some embodiments, the peptide may comprise a C-terminal sequence, e.g. a sequence at the C-terminus that does not comprise a domain defined above, e.g. a so-called C-terminal flanking sequence. In some embodiments, the peptide may comprise an N-terminal and C-terminal flanking sequence. The peptide may also comprise a C-terminal modification, e.g. an amide group. Thus, in some embodiments, the C-terminal residue may be amidated. In some preferred embodiments, the peptide comprises an amidated C-terminal arginine residue.

A flanking sequence may comprise from about 1-50 amino acids, such as about 1-40, 1-35, 1-30, 1-25, 1-20 etc. Thus, a flanking sequence may comprise 1, 2, 3, 4, 5, 6, 7, 8, 9, 10, 11, 12, 13, 14, 15, 16, 17, 18, 19, 20, 21, 22, 23, 24 or 25 amino acids, e.g. 1-40, 2-39, 3-38, 4-37, 5-36, 6-35, 7-34, 8-33, 9-32, 10-31, 11-30, 12-29, 13-28, 14-27, 15-26 amino acids or any combination thereof.

In some embodiments, the peptide of the invention may be in the form of a salt, i.e. a pharmaceutically acceptable salt. For instance, the peptide may be in the form of an acidic or basic salt, preferably an acidic salt. In some embodiments, the peptide is in a neutral salt form.

Pharmaceutically acceptable salts include pharmaceutical acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylat and benzene sulfonic acid salts.

Preferred salts include acidic salts, such as hydrochloride or organic acid addition salts such as acetate, alkyl sulfonate, arylsulfonate, maleate, fumarate, tartrate, citrate and lactate. In some embodiments, the peptide may be in the form of an acetate salt or derivative thereof, e.g. trichloroacetate (TCA), trifluoroacetate (TFA) etc. In some embodiments, the peptide may be stabilized by preparing it in the form of a salt, e.g. an acetate salt. Hydrochloride salts are particularly preferred.

“Pharmaceutically acceptable” as referred to herein refers to ingredients that are compatible with other ingredients used in the methods or uses of the invention as well as physiologically acceptable to the recipient.

The standard amino acid one letter code is used herein, so K stands for lysine (Lys), I stands for isoleucine (Ile) and so on.

In some embodiments, the peptide may comprise non-conventional or non-standard amino acids, i.e. in domains other than the APIM sequence (SEQ ID NO: 1). In some embodiments, the peptide may comprise one or more, e.g. 1, 2, 3, 4, 5 or more non-conventional amino acids, i.e. amino acids which possess a side chain that is not coded for by the standard genetic code, termed herein “non-coded amino acids”, and these are well-known in the art. For example, these may be selected from amino acids which are formed through metabolic processes such as ornithine or taurine, and/or artificially modified amino acids such as 9H-fluoren-9-ylmethoxycarbonyl (Fmoc), (tert)-(B)utyl (o)xy (c)arbonyl (Boc), 2,2,5,7,8-pentamethylchroman-6-sulphonyl (Pmc) protected amino acids, or amino acids having the benzyloxy-carbonyl (Z) group. In some embodiments, non-coded amino acids are present in more than one domain of the peptide.

In vitro and/or in vivo stability of the peptide may be improved or enhanced through the use of stabilising or protecting means known in the art, for example the addition of protecting or stabilising groups, incorporation of amino acid derivatives or analogues or chemical modification of amino acids. Such protecting or stabilising groups may for example be added at the N and/or C-terminus. An example of such a group is an acetyl group and other protecting groups or groups which might stabilise a peptide are known in the art.

The peptide of the invention will typically comprise only amino acids having the L-configuration, but one or more amino acids having the D configuration may be present. In some embodiments, the peptide contains 1, 2, 3, 4, 5 or more D-amino acids. In some embodiments, the D-amino acids are found in the motif, but in other embodiments, D-amino acids are present only outside of the motif. In a still further embodiment, D-amino acids may be found in more than one domain of the peptide. The peptide may be linear or cyclic, preferably linear.

In preferred embodiments, the peptide consists of L-amino acids. In yet a further preferred embodiment, the peptide consists of standard or coded L-amino acids.

As mentioned above, the peptide may comprise non-standard amino acids. Thus, in some embodiments the peptide may incorporate di-amino acids and/or β-amino acids. However, in preferred embodiments, at least the APIM motif domain, consists of α-amino acids. Most preferably, the peptide, i.e. all domains and optionally all flanking sequences, consists of α-amino acids.

The peptide defined herein comprises more than 5 amino acids, but the length of the peptide will depend on the size of the CPP sequence and on the number and size of other domains, e.g. linker domains, signal peptides, flanking sequences etc., if present. Thus, the term peptide refers to molecules containing a relatively small number of amino acids, i.e. less than 100, preferably less than 90, 80, 70, 60 or 50 amino acids. The peptide of the invention comprises at least 10, 11 or 12 amino acids, such as 13, 14, 15, 16, 17, 18, 19, 20, 21, 22, 23, 24 or 25 amino acids. Alternatively defined it comprises no more than 50, 45, 40, 35, 34, 33, 32, 31 or 30 amino acids. Representative subunit ranges thus include 12-50, 12-45, 12-40, 12-35, 12-30, 12-25, 12-23, 12-20, 12-18 etc, 12-30 and 12-40 being preferred. Further representative subunit ranges include 20-50, 21-45, 22-40, 23-35, 24-30, e.g. 25, 26, 27, 28, 29 or 30.

In some embodiments, the peptide may form part of a larger unit, e.g. it may be fused to a polypeptide to form a recombinant fusion protein or attached to a scaffold to form a peptide aptamer. Thus, fusion proteins or aptamers incorporating the peptide may also find utility in the uses and methods of the invention, i.e. in some embodiments the pharmaceutical composition may comprise a fusion protein or aptamer comprising the peptide defined above.

The pharmaceutical composition comprising peptide, fusion protein or aptamer or pharmaceutically acceptable salt thereof may be formulated together with at least one pharmacologically acceptable carrier or excipient.

The excipient may include any excipients known in the art, for example any carrier (vehicle) or diluent or any other ingredient or agent such as solvent (e.g. water), buffer (e.g., saline), antioxidant, chelator, solubilizer, emulsifier and/or preservative etc.

The pharmaceutical composition described herein may be administered systemically to the subject using any suitable means and the route of administration will depend on formulation of the pharmaceutical composition.

“Systemic administration” includes any form of non-local administration in which the agent is administered to the body at a site other than directly adjacent to, or in the local vicinity of, the carcinoma or sarcoma, resulting in the whole body receiving the administered peptide. Conveniently, systemic administration is by parenteral delivery (e.g. intravenous, intraperitoneal, intramuscular, or subcutaneous).

The pharmaceutical composition may be provided in any suitable form known in the art, for example as a liquid, suspension, emulsion, lyophilisate or any mixtures thereof.

In a preferred embodiment, the peptide is provided in a liquid pharmaceutical composition and methods for preparing such formulations are well-known in the art. Any such formulations may be used in the methods and uses of the invention.

In some embodiments, the pharmaceutical composition is a “ready to use” formulation that contains the peptide in dissolved or solubilized form and is intended to be used as such or upon further dilution in intravenous diluents. However, in some embodiments, the pharmaceutical composition may be provided in a solid form, e.g. as a lyophilisate, to be dissolved in a suitable solvent to provide a liquid formulation.

In a representative example, the peptide is stored as a lyophilisate and a pharmaceutical composition is prepared from the lyophilisate, e.g. by dissolving the peptide in a small volume of sterile water (e.g. 0.5-10 mL, such as about 1-5 mL) and optionally further diluted (e.g. with saline) to provide a suitable volume for infusion. In some preferred embodiments, the pharmaceutical composition is formulated for parenteral infusion or injection, preferably an intravenous or intraperitoneal infusion or injection.

In preferred embodiments, the pharmaceutical composition is formulated as an infusion (e.g. intravenous infusion). The volume and duration of the infusion can be determined by the skilled person and may depend on the characteristics of subject being treated, e.g. age, weight, sex etc. In a representative embodiment, the infusion may have a volume of about 100-750 mL, e.g. about 200-500 mL. In a further representative embodiment, the infusion may be administered over a period of about 30 minutes to about 8 hours, such as at least about 1 hour, e.g. 1-6 or 1-4 hours, e.g. about 1, 1.5, 2, 2.5 or 3 hours. In a further representative embodiment, the infusion may be administered over a period of about 30 minutes up to about 24 hours, such as up to about 23, 22, 21 or 20 hours, e.g. 1-24, 1-23, 1-20 or 1-16 hours.

The infusion flow rate readily may be determined by skilled person. In some embodiments, the infusion flow rate starts at about 5 mg/hr and increases approximately every 30 minutes until the required dose has been administered. A representative embodiment of the infusion flow rate is outlined in the Examples. In some embodiments, the maximum infusion rate should not exceed about 120 mg/hr and/or about 500 mL/hr. In some embodiments, doses of about 45 mg/m² or above (e.g. about 60 mg/m²) are administered in a volume of about 400-600 mL, e.g. about 500 mL.

The pharmaceutical composition is administered weekly to provide a dose of the peptide of about 15-65 mg/m² (e.g. 15-50 mg/m²) body surface area (BSA) per week, calculated as the free form of the peptide. In some embodiments, the pharmaceutical composition is administered weekly to provide a dose of the peptide of about 20-60 or 20-45 mg/m² body surface area (BSA) per week, calculated as the free form of the peptide, such as about 20, 30, 45 or 60 mg/m² body surface area (BSA) per week.

BSA (Body surface area) may be calculated, for example, using the Mosteller formula (√([height (cm)×weight (kg)]/3600)). Where necessary this may be converted to mg/kg by using a conversion factor for an average adult of 0.025 mg/kg=1 mg/m².

In some embodiments of the invention the pharmaceutical composition is administered weekly for at least three weeks, e.g. for 3, 4, 5, 6, 7, 8, 9 10 or more weeks (e.g. 20, 30, 40, 50 or more weeks). This administration may be in a single cycle or in total in multiple cycles.

Weekly administration typically refers regularly spaced administration, e.g. days, 1, 8 and 15 for a 3 week cycle. However, it will be evident that in order to achieve patient compliance, some flexibility may be required, i.e. weekly does not refer strictly to 7 day intervals. Thus, for instance, weekly administration may be days 1, 8±1 day and 15±1 day for a 3 week cycle, e.g. days 1, 7 and 15, days 1, 9 and 14, or days 1, 7 and 16 etc.

As referred to herein a “cycle” is a time period over which a particular treatment regime is applied and is generally repeated to provide cyclical treatment. The treatment in each cycle may be the same or different (e.g. different dosages, timings etc. may be used). In some embodiments, a cycle may be from 3-6 or 3-12 weeks in length, e.g. a 3, 4, 6, 9 or 12 week cycle. In some embodiments, a cycle may be about 1-6 months, i.e. weekly administration for about 1-6, e.g. 1-4 or 1-3 months, such as about 1 or 2 months (e.g. 3-26 weeks, such as about 3-16 or 3-12 weeks, e.g. about 4-8 weeks). In preferred embodiments, the cycle is repeated at least once. Thus, multiple cycles may be used, e.g. at least 2, 3, 4 or 5 cycles, e.g. 6, 7, 8, 9 or 10 (e.g. 10, 20, 30 or more) cycles. In some embodiments, treatment cycles may be continued until disease regression or progression occurs. In some embodiments, treatment cycles may be continued while the patient shows stable disease according to the RECIST criteria. In some embodiments, treatment may be restarted following a period of regression.

In some embodiments, treatment cycles may be delimited by a break in treatment, i.e. a period without weekly administration of the pharmaceutical composition. In some embodiments, the period between cycles is at least one week, e.g. 2, 3, 4 or more weeks. In some embodiments, the period between cycles is at least one month, e.g. 2, 3, 4 or more months.

However, in some embodiments, the second or subsequent treatment cycle may immediately follow the first or previous cycle. For instance, if the third weekly dose of the first cycle was administered on day 15±1 day, the first weekly dose of the second cycle may be administered on day 22±1 day.

In some embodiments of the invention, the patient may be subjected to other treatments prior to, contemporaneously with, or after the treatments of the present invention. For instance, in some embodiments, the patient may be treated with radiation therapy and/or surgery according to procedures known in the art.

Thus, in some embodiments, the patient to be treated has been or will be subjected to other treatments, e.g. with radiation therapy and/or surgery according to procedures known in the art. In some embodiments, the other treatment may be an immunotherapy, targeted therapy, hormone therapy, stem cell transplant or a combination thereof.

Thus, in some embodiments, the methods of the invention may comprise a further step of treating the subject with radiation therapy and/or surgery (prior to, contemporaneously with, or after treatments with the pharmaceutical composition of the present invention). Surgery may include resection of a carcinoma or sarcoma tumor.

In some embodiments, the pharmaceutical composition may contain one or more additional therapeutic agents or may be for administration with one or more additional therapeutic agents.

For instance, the inventors have found that administration of the pharmaceutical composition (particularly the first dose) may result in an allergic reaction (typically Grade 1 or 2) manifesting by one or more symptoms selected from a rash or itch (particularly at the site of administration), sweating, tachycardia, hives and fever, which may be treated by antihistamines.

Thus, in some embodiments, the pharmaceutical composition may contain or be administered (sequentially or simultaneously) with an agent for preventing or treating an allergic reaction, e.g. an antihistamine, a corticosteroid and/or an anti-inflammatory drug.

In some embodiments, the subject may be administered an agent for preventing or treating an allergic reaction prior to administration of the pharmaceutical composition of the invention. In some embodiments, the subject may be administered a corticosteroid (e.g. dexamethasone or a pharmacologically alternative corticosteroid at an equivalent dose), one or more histamine receptor antagonists (e.g. promethazine and/or ranitidine), an analgesic (e.g. acetaminophen) and/or an anti-leukotriene (e.g. montelukast), administered prior to administration of the pharmaceutical composition of the invention. In some embodiments, the subject may be administered a corticosteroid (e.g. dexamethasone or a pharmacologically alternative corticosteroid at an equivalent dose), one or more histamine receptor antagonists (e.g. promethazine and/or ranitidine), an analgesic (e.g. acetaminophen) and an anti-leukotriene (e.g. montelukast), administered prior to administration of the pharmaceutical composition of the invention.

While the inventors have advantageously found that the peptide of the invention is effective in the treatment of carcinomas and sarcomas when administered alone (e.g. in a first line treatment), in some embodiments the pharmaceutical composition may contain or be administered with one or more further therapeutic agent(s) useful in treating a carcinoma or sarcoma, e.g. a chemotherapeutic agent (e.g. a cytotoxic agent or small molecule targeted agent), immunotherapeutic agent (e.g. an immune checkpoint inhibitor, monoclonal antibody), a hormone or an agonist or antagonist thereof (e.g. gonadotropin-releasing hormone (GnRH) agonists, aromatase inhibitors, luteinizing hormone-releasing hormone (LHRH) agonists), stem cells or a combination thereof. For instance, in some embodiments, the peptide of the invention may be used in a combination therapy with one or more further therapy(ies) or therapeutic agent(s) (e.g. cytotoxic agent, immunotherapeutic agent etc.) as a second line treatment, i.e. to subjects refractory to an initial therapy, e.g. therapy with the peptide of the invention or other therapy or therapeutic agent alone. Thus, in some embodiments, the subject to be treated is refractory to one or more other therapies or therapeutic agents, e.g. chemotherapy or immunotherapy based therapies. In some embodiments, the peptide of the invention may be used alone or in a combination therapy with one or more further therapy(ies) or therapeutic agent(s) (e.g. cytotoxic agent, immunotherapeutic agent etc.) as a first line treatment.

In some preferred embodiments, the peptide of the invention is not administered as part of a combination therapy with another therapy or therapeutic agent, e.g. another chemotherapeutic agent or immunotherapy.

In some embodiments, the further or other therapy or therapeutic agent is an immunotherapy, such as an immunotherapy selected from immune checkpoint inhibitors, T-cell transfer therapy, antibody therapy, treatment vaccines or a combination thereof.

In some embodiments, the further or other therapy or therapeutic agent is a targeted therapy, such as a monoclonal antibody, e.g. an antibody-toxin conjugate, or a CAR-T cell therapy. Thus, in some embodiments, the further or other therapy or therapeutic agent is a targeted immunotherapy or immunotherapeutic agent.

In some embodiments, the further or other therapy or therapeutic agent is a chemotherapy or chemotherapeutic agent, such as a cytotoxic agent.

In some embodiments, cytotoxic agents (e.g. anti-cancer agents) described herein may be used to provide a sensitizing effect, in other words to enhance (or alternatively put to increase, augment, or potentiate) the effects of the peptide of the invention (e.g. in the treatment of a carcinoma or sarcoma), or to render a subject (or more particularly carcinoma or sarcoma cells or tumor(s) present in a subject) more susceptible to the effects of the peptide of the invention.

Thus, in some embodiments, the invention provides a pharmaceutical composition comprising a peptide or pharmaceutically acceptable salt thereof as a combined product with another therapeutic agent (e.g. a cytotoxic agent) for separate, simultaneous or sequential administration for use in treating a carcinoma or a sarcoma in a human subject, wherein the peptide comprises an amino acid sequence as set forth in SEQ ID NO: 1 and a cell penetrating peptide and wherein the pharmaceutical composition is systemically administered to the subject weekly to provide a dose of the peptide of about 15-65 mg/m² (e.g. 15-50 mg/m²) body surface area (BSA) per week, calculated as the free form of the peptide.

Alternatively viewed, the method of the invention further comprises administering another therapeutic agent (e.g. cytotoxic agent) to said subject, wherein said therapeutic agent (e.g. cytotoxic agent) is administered separately, simultaneously or sequentially to the pharmaceutical composition comprising the peptide as defined herein.

In some embodiments, the further therapeutic agent (e.g. cytotoxic agent) is an agent which is capable of inhibiting, suppressing the growth, viability and/or multiplication (replication/proliferation) of (e.g. killing) animal cells. In some embodiments, the further therapeutic agent (e.g. cytotoxic agent) is capable of inhibiting, suppressing the growth, viability and/or multiplication (replication/proliferation) of (e.g. killing) human carcinoma and/or sarcoma cells.

Included as cytotoxic agents are anti-neoplastic agents and any agent that may be indicated for an oncological application. Thus, included are agents used in chemotherapeutic treatment protocols (“chemotherapeutic agents” or “anti-cancer” agents).

Cytotoxic agents are typically grouped into different classes according to their mechanism of action and all of these classes are contemplated herein. Thus, the cytotoxic agent may be, for example, an alkylating agent, a cross-linking agent, an intercalating agent, a nucleotide analogue, an inhibitor of spindle formation, and/or an inhibitor of topoisomerase I and/or II. Other types or classes of agent include anti-metabolites, plant alkaloids and terpenoids, or an anti-tumor antibiotic.

Alkylating agents modify DNA by alkylating nucleosides, which leads to the prevention of correct DNA replication. Nucleotide analogues become incorporated into DNA during replication and inhibit DNA synthesis. Inhibitors of spindle formation disturb spindle formation, leading to the arrest of mitosis during metaphase. Intercalating agents intercalate between DNA bases, thereby inhibiting DNA synthesis. Inhibitors of topoisomerase I or II affect the torsion of DNA, thereby interfering with DNA replication.

Suitable cytotoxic agents are known in the art, but by way of example include actinomycin D, bortezeomib, BCNU (carmustine), BI 2536, buparlisib, carboplatin, CCNU, campothecin (CPT), cantharidin, cisplatin, combretastatin A4, CUDC-907, cyclophosphamide, cytarabine, dasatanib, dacarbazine, dactosilib, daporinad, daunorubicin, docetaxel, doxorubicin, duvelisib, DTIC, elesclomol, epirubicin, etoposide, gefinitib, gemcitabine, idelalisib, ifosamide, ispinesib, irinotecan, ionomycin, luminespib, melphalan, methotrexate, mitomycin C (MMC), mitozantronemercaptopurine, molibresib, oxaliplatin, obatoclax, paclitaxel (taxol), PARP-1 inhibitor, pelitinib, perifosine, PX-866, sepantronium bromide, SB-743921, taselisib, taxotere, temozolomide (TZM), teniposide, topotecan, trametinib, treosulfane triptolide, umbralisib, vinorelbine, vincristine, vinblastine, volasertib, voxtalisib, 5-azacytidine, 5,6-dihydro-5-azacytidine and 5-fluorouracil. Any of the aforementioned cytotoxic agents may be used in the combination therapies of the invention.

The cytotoxic agents for use in combination with the pharmaceutical composition comprising the peptide as defined herein may be provided in pharmaceutical compositions as defined above and may be administered as defined above. In some embodiments, the pharmaceutical compositions comprising cytotoxic agents may be formulated for parenteral administration. Thus, the compositions may comprise pharmaceutically acceptable excipients, solvents and diluents suitable for such formulations, e.g. intravenous bolus or injection.

The skilled person will be aware of suitable dosage ranges for any given cytotoxic agent. In preferred embodiments, the cytotoxic agent is present in the pharmaceutical composition, or administered to the subject, in its typical dose range.

Preferred aspects according to the invention are as set out in the Examples in which one or more of the parameters or components used in the Examples may be used as preferred features of the methods described hereinbefore.

The invention will now be further described with reference to the following non-limiting Examples and FIGURE.

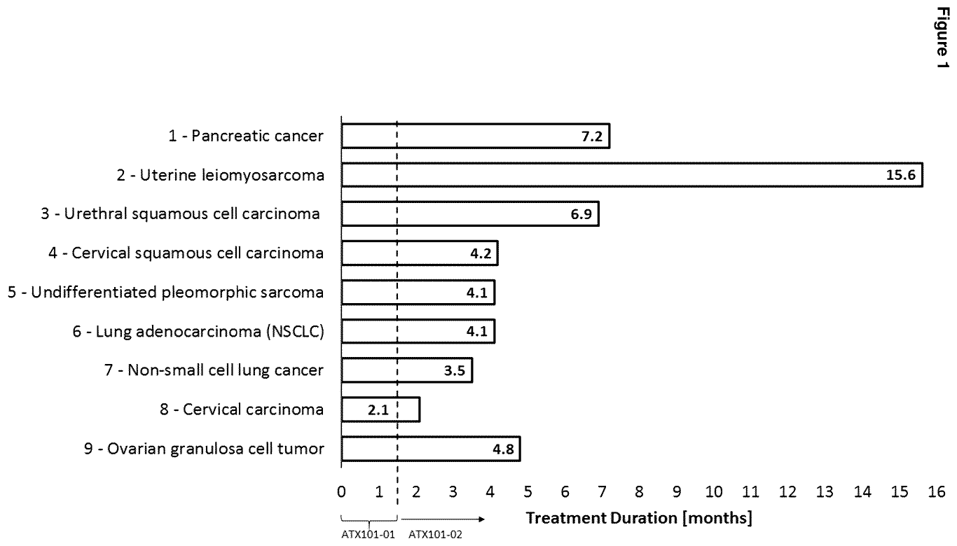

FIG. 1 shows a Swimmer plot of the duration of treatment of subjects treated in the long-term follow-up study (ATX-101-02) as set out in Tables 2-4. The FIGURES in each bar show the total number of months during which the treatment resulted in stable disease and until final study discontinuation due to disease progression or withdrawal. The dotted line indicates the end of the first treatment part after 6 weeks (ATX-101-01), the timepoint of the first tumor assessment as per RECIST V1.1. All patients had stable disease at this point in time and moved to the long-term follow-up treatment (study ATX101-02).

### EXAMPLES

### Example 1—Clinical Study of ATX-101 (SEQ ID NO: 914) in Various Carcinoma and Sarcoma Patients

### Clinical Study Details

A Phase I, open-label, single arm, safety and tolerability study was conducted (study ATX101-01) which evaluated escalating dose cohorts of ATX-101 (SEQ ID NO: 914) in patients with advanced solid tumors. The study was designed to systematically assess safety and tolerability, and to identify the Maximum Tolerated Dose (MTD) and recommended Phase II dose for ATX-101. Pharmacokinetics and preliminary efficacy (anti-tumor activity) were also assessed.

If at the end of the 6-week Phase I study (study ATX101-01) the patient's tumor didn't show signs of progression, treatment could be continued in a long-term follow-up study (study ATX101-02) until disease progression or other reasons for treatment discontinuation, e.g. withdrawal of consent.

### Eligibility for Inclusion in the Clinical Study

### Inclusion Criteria

1. Women or men≥18 years of age
2. Signed written informed consent
3. Advanced disease for which conventional anti-tumor treatment has been exhausted or has been refused
4. Measurable or non-measurable (but radiologically evaluable) disease on CT/MRI scan with at least one lesion outside previously irradiated areas
5. Have an ECOG Performance status 0-2
6. Life expectancy of at least 3 months
7. Meet the following laboratory requirements:
   Absolute neutrophil count (ANC)≥1.5×10⁹/L
   Platelet count≥75×10⁹/L
   aPTT/PT≤1.5×ULN
   Total bilirubin level≤1.5×ULN.
   AST and ALT≤2.5×ULN (≤5×ULN if liver metastasis present).
   Creatinine≤1.5×ULN
   Albumin≥30 g/L
8. Women of child-bearing potential (WOCBP) must use highly effective contraceptive measures (failure rate of <1% per year when used consistently and correctly) and intend to continue use of contraception for at least 1 month following the last infusion. Highly effective contraceptive measures could include: combined (oestrogen and progestogen containing) hormonal contraception associated with inhibition of ovulation, progestogen-only hormonal contraception associated with inhibition of ovulation, intrauterine device, intrauterine hormone releasing system, bilateral tubal occlusion, vasectomized partner, and sexual abstinence
9. Males who are not surgically sterile must use a condom through to study completion and for 30 days after the last treatment administration, unless they have a female partner who is surgically sterile or post-menopausal. They must refrain from fathering a child during this time.

### Exclusion Criteria

1. Have received an investigational drug within 4 weeks (6 weeks for investigational immunotherapy agents) prior to study drug administration or is scheduled to receive such treatment during the defined treatment or the post-treatment period of the study
2 Concurrent anticancer treatment (e.g., cytoreductive therapy, radiotherapy except for palliative bone-directed radiotherapy, immune therapy, or cytokine therapy except for erythropoietin) within 21 days or 5× (five times) their half-lives (whichever is shorter) before the first dose of trial treatment
3. Use of hormonal agents within 7 days before start of trial treatment, except for patients with castration-resistant prostate cancer (CRPC), who may remain on treatment with luteinizing hormone-releasing hormone agonists or antagonists a. Note: Patients receiving bisphosphonate or denosumab are eligible provided that treatment was initiated ≥14 days before first dose of treatment.
4. Anticipated requirement for surgery or initiation of anti-cancer therapy during the study period
5. Have not recovered from AEs (>CTCAE Grade 2 other than alopecia) due to agent(s) administered more than 4 weeks earlier
6. Cardiac failure (per New York Heart Association [NYHA] functional classification) of >Grade 2.
7. Evidence or history of clinically significant cardiac disease including congestive heart failure, unstable angina, acute myocardial infarction or cerebrovascular accident within the last six months, and symptomatic arrhythmia requiring therapy (with the exception of extra systoles or minor conduction abnormalities and controlled and well-treated chronic atrial fibrillation).
8. QTcF>460 ms
9. Active central nervous system (CNS) metastases. Patients with known CNS metastases must have received previous radiotherapy or surgery at least two weeks prior to receiving ATX-101. Any residual neurological deficit must be stable off corticosteroids
10. Lymphangitic carcinomatosis
11. Leptomeningeal involvement
12. Major surgery within 3 weeks of screening
13. Current acute or chronic disease, other than the study indication, that would increase the expected risk of exposure to the investigational product or would be expected to interfere with the planned evaluations, in the judgment of the Investigator
14. Breastfeeding or pregnant as confirmed by a positive serum beta human chorionic gonadotropin (β-HCG) pregnancy test at screening or at subsequent clinic visits
15. Unwilling or unable to follow protocol requirements
16. Known positive status of Human immunodeficiency virus (HIV) and/or active Hepatitis B or C. In patients with a history of Hepatitis B or Hepatitis C infection, resolution of infection must be demonstrated by negative serology for Hepatitis B surface antigen (HBsAg) and Hepatitis C virus (HCV) ribonucleic acid (RNA) must be demonstrated at least 6 weeks following antiviral therapy
17. History of severe allergy (requiring hospital care), severe reaction to any drug, or any known or suspected allergies or sensitivities to the study drug constituents
18. Inadequate venous access to allow collection of blood samples

### ATX-101 Drug Substance

The peptide drug substance is an amorphous material, with no known crystalline or polymorphic forms, freely soluble in water and aqueous media. The product was available as a hydrochloride salt (chloride counter-ion is ionically bound to the basic side chains of the peptide; 18 mol/mol peptide, theoretically; Molecular formula: C₁₅₈H₂₈₅N₇₁O₂₉S, 18 HCl; Relative molecular mass: 4320.9. Free base: 3673.3).

### ATX-101 Drug Product

The peptide was provided as a sterile lyophilisate for reconstitution and dilution prior to injection. The lyophilisate was presented in single use colourless vials of 5 or 80 mg with rubber stoppers and aluminium flip-off seals with plastic discs stored at −20° C.±5° C. protected from light. Prior to dosing, the study drug was removed from the freezer and reconstituted with 1 ml (5 mg) or 4 mL (80 mg) sterile water for injection. To aid with reconstitution prior to intravenous infusion, the vial was gently swirled, not shaken, prior to dilution in normal saline in the appropriate volume (e.g. 100 mL, 250 mL and 500 mL) in infusion bags.

### Treatments and Dose Levels

Four dose levels (20, 35, 45 and 60 mg/m² of body surface area calculated based on the height and weight of patients) of intravenous ATX-101 (net peptide, excluding the mass of the salt component) were tested in the Phase I study (study ATX101-01). ATX-101 was administered via IV infusion over at least 1 hour either using a constant infusion rate or an adaptable infusion rate scheme starting with 5 mg/hr and increasing the infusion flow rate every 30 minutes, as outlined in the infusion rate table below (Table 1). The maximum infusion rate did not exceed 120 mg/hr and/or 500 mL/hr. Doses of 45 and 60 mg/m² were administered in 500 mL.

TABLE 1
Infusion Rate

| Infusion | Infusion | Cumulative | Dose Infused | Cumulative |
|---|---|---|---|---|
| Rate | Time | Infusion Time | for timepoint | Dose infused |
| (mg/hr) | (min) | (min) | (mg) | (mg) |
| 5 | 30 | 30 | 2.5 | 2.5 |
| 20 | 30 | 60 | 10 | 12.5 |
| 60 | 30 | 90 | 30 | 42.5 |
| 90 | 30 | 120 | 45.0 | 87.5 |
| 120 | — | — | — | — |

Treatment was administered weekly in cycles of 21-day duration, with a single IV infusion of ATX-101 on Day 1, 8 and 15 of each cycle. Dosing of patients occurred weekly for up to two cycles (i.e. 2×21 days=6 weeks).

Following the initial 6 weeks of treatment in the Phase I study (study ATX101-01), patients could continue the treatment in the long-term follow-up study (study ATX101-02). These patients, that received the same dose regimen as in study-01, were treated for up to 15.6 months.

### Assessments Performed

Safety: Incidence, severity, and duration of treatment-emergent adverse events (TEAEs) and treatment related TEAEs were assessed according to Common Terminology Criteria for Adverse Events (CTCAE) v4.03. For cases were CTCAE v4.03 did not apply to an adverse event (AE), the AE was used as based on a grading of mild, moderate, or severe.

Efficacy: Tumor assessment was performed as per Response Evaluation Criteria In Solid Tumors (RECIST V1.1., https://ctep.cancer.gov/protocolDevelopment/docs/recist_guideline.pdf).

Tumor imaging was performed using CT or MRI of the chest/abdomen/pelvis (plus other regions as required for specific tumor types). The assessment was performed at baseline and following up to six weekly treatments with ATX-101 according to RECIST V1.1 (end of study ATX101-01). In patients that have been treated for a longer period (i.e. in the long-term follow-up study ATX101-02), further assessments have been performed every 3 months (+14 days). In general, lesions detected at baseline were to be followed using the same imaging methodology and the same imaging equipment at the subsequent tumor evaluation visit.

Clinical signs of tumor progression have been checked during the entire treatment period. This included specific symptoms, physical examinations, laboratory values and other assessments.

For each patient, the responsible investigator use the most appropriate of the above mentioned measures to evaluate the patient's tumor status. In patients with clinical signs of progression, unscheduled tumor scans and assessments were recommended. In general, measure(s) chosen for the tumor assessment of the individual patient were to be consistent during the trial and corresponded to measures used to qualify the patient for enrolment.

Based on the aforementioned assessments, the following parameters were evaluated: Disease Control Rate (DCR): defined as the proportion of patients that show absence of signs of disease progression based on RECIST V1.1 during the entire study participation.

The Objective Response Rate (ORR): defined as the proportion of patients who achieve complete response (CR) or partial response (PR) based on RECIST V1.1 criteria.

### Patient Population

Twenty-two patients were treated in the Phase I study. Ten of the 22 patients (45%) showed absence of signs of disease progression after the first 6 weeks of treatment (end of study ATX101-01). Nine of these 10 patients were enrolled in the long-term follow-up study and are part of the efficacy considerations presented below. Table 2 summarizes the diseases of these 9 patients, their tumor status at study entry and the ATX-101 doses they received. All patients had progressive tumors at study entry, except patient #1 who had stable disease.

TABLE 2
Patients treated in the long-term follow-up study (ATX101-02)

| # | Cancer type | Status at study entry | Dose used |
|---|---|---|---|
| 1 | Pancreatic adenocarcinoma | Stable | 20 mg/m² |
| 2 | Uterine Leiomyosarcoma | Progressive | 20 mg/m² |
| 3 | Urethral squamous cell carcinoma | Progressive | 20 mg/m² |
| 4 | Cervical squamous cell carcinoma | Progressive | 20 mg/m² |
| 5 | Undifferentiated pleomorphic | Progressive | 30 mg/m² |
| | Sarcoma | | |
| 6 | Non-small cell lung cancer | Progressive | 45 mg/m² |
| 7 | Non-small cell lung cancer | Progressive | 45 mg/m² |
| 8 | Cervical carcinoma | Progressive | 60 mg/m² |
| 9 | Ovarian granulosa cell tumor | Progressive | 60 mg/m² |

Table 3 presents the total number of anti-cancer treatments (including surgery, chemotherapy and radiotherapy) prior to study entry for patients treated in the long-term follow-up study. Based on their advanced disease status, most patients underwent a large number of prior treatments.

TABLE 3
Number of anti-cancer treatments prior to study entry
for patients treated in the long-term follow-up study

| # | Cancer type | Number of prior treatments |
|---|---|---|
| 1 | Pancreatic adenocarcinoma | 4 |
| 2 | Uterine Leiomyosarcoma | 4 |
| 3 | Urethral squamous cell carcinoma | 2 |
| 4 | Cervical squamous cell carcinoma | 9 |
| 5 | Undifferentiated pleomorphic Sarcoma | 7 |
| 6 | Non-small cell lung cancer | 8 |
| 7 | Non-small cell lung cancer | 15 |
| 8 | Cervical carcinoma | 7 |
| 9 | Ovarian granulosa cell tumor | 9 |

### Safety Data

ATX-101 had a favorable safety profile in all 22 patients treated in the Phase 1 studies (studies ATX101-01 and -02). No treatment related deaths and no Dose Limiting Toxicities (DLTs) were reported. There was no treatment related serious adverse event or severe adverse event. There was no treatment discontinuation due to treatment related toxicity. Treatment related adverse events were only mild to moderate.

The most common-treatment related events were infusion-related reactions (IRR) Grade 1 or 2, observed in 73% of patients. These are a kind of allergic reactions presented by itchiness, redness, urticaria, fever, rash, swelling, flushing and hives. Symptoms resolved rapidly upon discontinuation of the infusion with or without symptomatic treatment with antihistamines and/or corticosteroids. In the majority of patients, the infusion of ATX-101 could be safely restarted and the treatment was completed. IRR were reported during the first but also later infusions. They did not worsen after repeated doses of ATX-101. Two measures have been implemented to manage the IRR: a stepwise increase of the infusion rate for each infusion (see Table 1) and a mandatory premedication consisting of dexamethasone, paracetamol (acetaminophen), montelukast and histamine receptor antagonists.

### Safety Summary

ATX-101 can be safely administered as monotherapy at all investigated doses. Treatment related adverse events were only mild to moderate in terms of severity. IRR have been observed in most patients, but they were manageable without causing severe or life-threatening situations.

### Efficacy Data

Table 4 and FIG. 1 present the total duration of treatment with ATX-101 for the patients treated longer than 6 weeks. Three patients had stable disease and one patient had no tumor assessment at end of treatment, the remaining 5 patients had progressive disease. The median duration of treatment was 4.2 [2.1-15.6] months.

TABLE 4
Treatment duration and efficacy outcome for patients
treated in the long-term follow-up study (ATX-101-02)

| | | Duration of | |
|---|---|---|---|
| | | treatment | Outcome at end of |
| # | Cancer type | (months) | treatment |
| 1 | Pancreatic adenocarcinoma | 7.2 | Stable disease |
| 2 | Uterine Leiomyosarcoma | 15.6 | Stable disease |
| 3 | Urethral squamous cell | 6.9 | Progressive disease |
| | carcinoma | | |
| 4 | Cervical squamous cell | 4.2 | Progressive disease |
| | carcinoma | | |
| 5 | Undifferentiated | 4.1 | Progressive disease |
| | pleomorphic Sarcoma | | |
| 6 | Non-small cell lung cancer | 4.1 | Stable disease |
| 7 | Non-small cell lung cancer | 3.5 | Progressive disease |
| 8 | Cervical carcinoma | 2.1 | Progressive disease |
| 9 | Ovarian granulosa cell | 4.8 | No tumor |
| | tumor | | assessment |

Short case reports for the 9 patients that have been treated in the long-term follow-up study are provided below.

### Case 1: Pancreatic Cancer

This 67-year-old male patient was diagnosed with metastatic pancreatic cancer in May 2018. He was treated with gemcitabine plus nab-paclitaxel until August 2018. The patient was enrolled in the clinical study in October 2018. At this time, the disease was stable and showed metastases in the liver (segment 4/5) and abdomen (coeliac axis). The tumor remained stable during the first 6 weeks of treatment with weekly infusions of 20 mg/m² ATX-101. Consequently, the treatment was continued. After a total treatment duration of 7.2 months the treatment was discontinued due to worsening of the performance status in the absence of progressive disease.

### Case 2: Uterine Leiomyosarcoma

This woman was diagnosed with uterine leiomyosarcoma in August 2015 when she was 45 years old. Prior to ATX-101 treatment, she received four anticancer treatments including hormone therapies (tamoxifen, letrozole, medroxyprogesterone) and chemotherapy (gemcitabine plus docetaxel).

At the time of study entry, in October 2018, the sarcoma was progressive, and metastasized with lesions in the lung (right middle and left lower lobe) and lymph nodes (left external iliac). Following 6 weekly infusions of 20 mg/m² ATX-101, the tumor growth was stopped, and the disease became stable according to the common RECIST tumor assessment criteria. The weekly infusions were continued over a total of 15.6 months with regular tumor assessments. The disease remained stable with no signs of tumor growth. The patient decided to interrupt the treatment, but tumor control continued. Two years after ATX-101 treatment start, in November 2020, no signs of tumor growth have been observed.

### Case 3: Urethral Squamous Cell Carcinoma

This male patient was 61 years old when he was diagnosed with urethral carcinoma in December 2017. Prior to study entry, he has been treated with a chemotherapy combination consisting of paclitaxel, ifosfamide and cisplatin. This combination treatment was stopped in June 2018 because the tumor became progressive during the treatment.

In January 2019, the patient was enrolled in the clinical study. At this time, the progressive carcinoma spread to lymph nodes (left & right inguinal and right external iliac). Following the first 6 weeks of 20 mg/m² weekly ATX-101 infusions, the patient showed no signs of disease progression indicating that the disease had been stabilized. The patient has been treated for a total of 6.9 months when the tumor became progressive, and the treatment was stopped.

### Case 4: Cervical Squamous Cell Carcinoma

This woman was diagnosed with cervical carcinoma at the age of 32 in January 1985. She received surgeries, chemotherapies, and radiotherapies as anticancer treatments prior to ATX-101 treatment. Chemotherapies included combinations of carboplatin plus paclitaxel, cisplatin plus radiotherapy (pelvis), carboplatin plus paclitaxel plus bevacizumab, as well as monotherapies with cisplatin and finally an experimental anti-PD-1 agent. The latter treatment was the most recent treatment before study entry, and it was discontinued due to disease progression in August 2018.

The patient was enrolled in the clinical study in January 2019. At this time, the tumor metastasized to the right iliac soft tissue and a left supraclavicular lymph node. After the first 6 weeks of weekly 20 mg/m² ATX-101 infusions, the tumor became stable and the treatment was continued. Overall, the patient was treated over 4.2 months when the disease progressed, and the treatment was discontinued.

### Case 5: Undifferentiated Pleomorphic Sarcoma

This male patient was diagnosed with metastatic sarcoma at the age of 63 in December 2010. Prior to ATX-101 treatment, he underwent left lower limb amputation, received gemcitabine plus docetaxel, pazopanib, radiotherapy (left hip, right arm), doxorubicin and right upper limb amputation. Chemotherapy with pazopanib was the most recent systemic therapy which was discontinued due to disease progression in December 2017.

The patient was enrolled the clinical study in April 2019. At this time, the sarcoma showed lesions in lung and thigh. Following 6 weekly intravenous ATX-101 doses of 30 mg/m², the patient's tumor showed no signs of disease progression and the treatment was continued for a total of 4.1 months, when progressive disease was diagnosed.

### Case 6: Lung Adenocarcinoma (Non-Small Cell Lung Cancer)

This male patient was diagnosed with metastatic lung adenocarcinoma at the age of 56 in October 2013. He underwent right upper lung lobectomy followed by radiotherapy in 2013 and left upper lung lobectomy in 2015. In addition, he received treatment lines of chemotherapy: two times cisplatin plus vinorelbine, nivolumab, pemetrexed and carboplatin plus gemcitabine. The last therapy, carboplatin plus gemcitabine, was discontinued due to disease progression in April 2019.

The patient was enrolled in the clinical study in September 2019. At this time, the disease manifested itself with multiple lesions in the lungs and adrenal glands (left and right). The patient was treated with weekly infusions of 45 mg/m² ATX-101. After 6 weeks treatment the disease stabilized, and the ATX-101 treatment was continued. Overall, the patients received infusions over 4.1 months. The treatment was discontinued although no signs of disease progression have been reported.

### Case 7: Non-Small Cell Lung Cancer (NSCLC)

This female patient was diagnosed with NSCLC at the age of 64 in September 2017. The patient underwent several local therapies, including right upper/middle lung lobectomy, brain stereotactic radiosurgery, and radiotherapies of several regions. She received 3 lines of systemic treatments: carboplatin plus pemetrexed, atezolizumab, and an experimental agent (PD-1/CTLA-4 bispecific antibody). The treatment with the experimental agent was discontinued in August 2019 due to disease progression. Between this discontinuation and study entry, the patient received radiation therapy of skull and brain.

The patient was enrolled in the clinical study in September 2019. At this time, the disease showed different lesions in the kidney, left adrenal gland, thyroid gland and lung. The patient received weekly infusions of 45 mg/m² ATX-101. After 6 weeks of treatment the disease has been stabilized. The patient continued the treatment over a total of 3.5 months when the disease became progressive and the treatment was discontinued.

### Case 8: Cervical Cancer

This woman was diagnosed with cervical cancer at the age of 47 in June 2018. The tumor was resected and the area irradiated. In 2019 three different systemic anticancer treatments have been started: chemotherapy with carboplatin plus paclitaxel plus bevacizumab, followed by bevacizumab maintenance therapy and finally a treatment with an investigational agent (anti-PD-1 antibody). The latter treatment was discontinued due to disease progression in March 2020.

The patient was enrolled in the clinical study in May 2020. At this time, the tumor metastasized to the soft tissue in the pelvic region. The patient received weekly infusions of 60 mg/m² ATX-101. After 6 weeks of treatment the disease has been stabilized. The patient continued the treatment over a total of 2.1 months when the treatment was early terminated due to urosepsis and tumor blockage of a urethral stent which was considered clinical disease progression. No tumor imaging was performed.

### Case 9: Ovarian Granulosa Cell Tumor

This woman was diagnosed with ovarian carcinoma at the age of 48 in December 2009. Prior to recruitment in the study, she received a number of different anti-cancer treatments: 5 debulking surgeries, two hormonal therapies and two treatment lines of chemotherapy combinations (etoposide plus ifosfamide plus cisplatin and carboplatin plus gemcitabine). The most recent systemic therapy prior to ATX-101, carboplatin plus gemcitabine, was stopped after almost 2 years of treatment due to disease progression in November 2017.

In February 2020, the patient received the fifth debulking surgery and was then recruited into the study in August 2020. The patient has been treated over 5 months with weekly 60 mg/m² infusions of ATX-101 without signs of disease progression. The treatment was discontinued without evidence of disease progression because another debulking surgery has been scheduled.

### Efficacy Summary

95% (n=21) of patients recruited in the Phase I study had progressive disease at study entry. 45% of all patients (n=10) had stable disease after the first 6 weeks of ATX-101 treatment. Nine patients (42% of the total patient population) continued treatment with a total treatment duration of a median of 4.2 months. Only 5 of these 9 patients discontinued treatment due to disease progression. It can be concluded that the median progression free survival in these patients exceeds 4.2 months. Considering the tumor status at treatment start, this stabilization of disease could be attributed to ATX-101 activity.

### Overall Clinical Conclusions

ATX-101 is a first in class compound that is well tolerated when administered as a weekly infusion. The only identified ATX-101-related, mild to moderate adverse events are infusion related reactions, which are easily manageable.

In a remarkable portion of heavily pre-treated cancer patients that had no further standard treatment options, ATX-101 could stabilize the disease over a clinically meaningful time. This effect was completely unexpected in the context of the Phase I study for which the objective was to determine the safety and tolerability of the compound. Prior to the study, it was expected that doses in excess of 60 mg/m² would be tested until a maximum tolerated dose was determined and that only these higher doses would provide effective treatment. Notably, the stabilization effect was observed already at the lowest tested dose of 20 mg/m² but was also evident at all other investigated dose levels (30, 45 and 60 mg/m²). Surprisingly, no dose-dependent effects were observed indicating that the dosage range of about 15-65 mg/m² represents an effective treatment for various tumor types.

The Phase I data indicate that the risk-benefit ratio for the patients is in favor of the benefit. It was particularly surprising that the tested dosage regimen was effective in the patients recruited to the Phase I study given their predominant status as being refractory to other treatments. Moreover, the fact that the effects were observed in a variety of disease settings supports the utility of ATX-101 in the claimed dosage range and patient group, and the characteristics of ATX-101 support the further clinical development as both mono- and combination therapy.

1-22. (canceled)

23. A method of treating a non-small cell lung cancer in a human subject in need thereof, the method comprising administering a pharmaceutical composition comprising a peptide or a pharmaceutically acceptable salt thereof to the subject, wherein the peptide comprises an amino acid sequence as set forth in SEQ ID NO: 914 and wherein the pharmaceutical composition is systemically administered to the subject weekly to provide a dose of the peptide of about 15-65 mg/m² body surface area (BSA) per week, calculated as the free form of the peptide.

24. The method of claim 23, wherein the dose of the peptide is about 30-65 mg/m² BSA per week, calculated as the free form of the peptide.

25-27. (canceled)

28. The method of claim 23, wherein the dose of the peptide is about 45-65 mg/m² BSA per week, calculated as the free form of the peptide.

29. The method of claim 23, wherein the non-small cell lung cancer is a large cell carcinoma.

30. The method of claim 23, wherein the non-small cell lung cancer is an adenocarcinoma.

31. The method of claim 23 comprising administering the pharmaceutical composition to the subject weekly for a treatment cycle of at least three weeks.

32. The method of claim 31, wherein the treatment cycle is repeated at least once.

33. The method of claim 23 comprising administering the pharmaceutical composition parenterally.

34. The method of claim 23 comprising administering the pharmaceutical composition by intravenous infusion for at least about 1 hour.

35. The method of claim 23, wherein the peptide is provided as a hydrochloride salt.

36. The method of claim 23 comprising administering the pharmaceutical composition to the subject weekly in multiple treatment cycles.

37. The method of claim 36, wherein there is a break between treatment cycles.

38. The method of claim 37, wherein the period between treatment cycles is at least one week.


SEQUENCE LISTING (920 sequences)

SEQ ID NO: 1 - protein, 5 aa, synthetic construct
  REGION 1..5: note = PCNA interacting motif
RWLVK

SEQ ID NO: 2-4 - skipped (3 entries)

SEQ ID NO: 5 - protein, 16 aa, synthetic construct
  REGION 1..16: note = Penetratin
RQIKIWFQNRRMKWKK

SEQ ID NO: 6 - protein, 7 aa, synthetic construct
  REGION 1..7: note = Penetratin derivative
RRMKWKK

SEQ ID NO: 7 - protein, 8 aa, synthetic construct
  REGION 1..8: note = Penetratin derivative
  MOD_RES 1: note = Nle
XRRMKWKK

SEQ ID NO: 8 - protein, 9 aa, synthetic construct
  REGION 1..9: note = Penetratin derivative
  MOD_RES 2: note = Nle
QXRRMKWKK

SEQ ID NO: 9 - protein, 10 aa, synthetic construct
  REGION 1..10: note = Penetratin derivative
  MOD_RES 3: note = Nle
FQXRRMKWKK

SEQ ID NO: 10 - protein, 7 aa, synthetic construct
  REGION 1..7: note = Penetratin derivative
RREKWKK

SEQ ID NO: 11 - protein, 7 aa, synthetic construct
  REGION 1..7: note = Penetratin derivative
RRQKWKK

SEQ ID NO: 12 - protein, 7 aa, synthetic construct
  REGION 1..7: note = Penetratin derivative
KRMKWKK

SEQ ID NO: 13 - protein, 7 aa, synthetic construct
  REGION 1..7: note = Penetratin derivative
RKMKWKK

SEQ ID NO: 14 - protein, 7 aa, synthetic construct
  REGION 1..7: note = Penetratin derivative
  MOD_RES 3: note = Orn
RRXKWKK

SEQ ID NO: 15 - protein, 7 aa, synthetic construct
  REGION 1..7: note = Penetratin derivative
RRMKQKK

SEQ ID NO: 16 - protein, 7 aa, synthetic construct
  REGION 1..7: note = Penetratin derivative
RRMKWFK

SEQ ID NO: 17 - protein, 7 aa, synthetic construct
  REGION 1..7: note = Penetratin derivative
  MOD_RES 2: note = Orn
RXRKWKK

SEQ ID NO: 18 - protein, 7 aa, synthetic construct
  REGION 1..7: note = Penetratin derivative
RRMWKKK

SEQ ID NO: 19 - protein, 7 aa, synthetic construct
  REGION 1..7: note = Penetratin derivative
RRMKKWK

SEQ ID NO: 20 - protein, 16 aa, synthetic construct
  REGION 1..16: note = D-penetratin
RQIKIWFQNRRMKWKK

SEQ ID NO: 21 - protein, 18 aa, synthetic construct
  REGION 1..18: note = Pegelin (SynB)
RGGRLSYSRRRFSTSTGR

SEQ ID NO: 22 - protein, 13 aa, synthetic construct
  REGION 1..13: note = HIV-TAT
GRKKRRQRRRPPQ

SEQ ID NO: 23 - protein, 11 aa, synthetic construct
  REGION 1..11: note = Residues 47-57 of HIV-TAT
YGRKKRRQRRR

SEQ ID NO: 24 - protein, 33 aa, synthetic construct
  REGION 1..33: note = VP22
DAATATRGRSAASRPTERPRAPARSASRPRRVD

SEQ ID NO: 25 - protein, 18 aa, synthetic construct
  REGION 1..18: note = MAP
KLALKLALKALKAALKLA

SEQ ID NO: 26 - protein, 27 aa, synthetic construct
  REGION 1..27: note = Transportan
GWTLNSAGYLLGKINLKALAALAKKIL

SEQ ID NO: 27 - protein, 21 aa, synthetic construct
  REGION 1..21: note = Transportan-10
AGYLLGKINLKALAALAKKIL

SEQ ID NO: 28 - protein, 30 aa, synthetic construct
  REGION 1..30: note = KALA
WEAKLAKALAKALAKHLAKALAKALKACEA

SEQ ID NO: 29 - protein, 21 aa, synthetic construct
  REGION 1..21: note = Pep-1
KETWWETWWTEWSQPKKKRKV

SEQ ID NO: 30 - protein, 21 aa, synthetic construct
  REGION 1..21: note = Pep-2
KETWFETWFTEWSQPKKKRKV

SEQ ID NO: 31 - protein, 27 aa, synthetic construct
  REGION 1..27: note = MPG
GALFLGFLGAAGSTMGAWSQPKSKRKV

SEQ ID NO: 32 - protein, 19 aa, synthetic construct
  REGION 1..19: note = Vectocell peptide
VKRGLKLRHVRPRVTRMDV

SEQ ID NO: 33 - protein, 14 aa, synthetic construct
  REGION 1..14: note = Vectocell peptide
SRRARRSPRHLGSG

SEQ ID NO: 34 - protein, 16 aa, synthetic construct
  REGION 1..16: note = Vectocell peptide
LRRERQSRLRRERQSR

SEQ ID NO: 35 - protein, 22 aa, synthetic construct
  REGION 1..22: note = Vectocell peptide
GAYDLRRRERQSRLRRRERQSR

SEQ ID NO: 36 - protein, 29 aa, synthetic construct
  REGION 1..29: note = Wr-T transporter
  REGION 22..29: note = variant - /REPLACE= D enantiomer arginine
KETWWETWWTEWWTEWSQGPGXXXXXXXX

SEQ ID NO: 37 - protein, 7 aa, synthetic construct
  REGION 1..7: note = R7
RRRRRRR

SEQ ID NO: 38 - protein, 9 aa, synthetic construct
  REGION 1..9: note = HIV-TAT peptide
RKKRRQRRR

SEQ ID NO: 39 - protein, 8 aa, synthetic construct
  REGION 1..8: note = R8
RRRRRRRR

SEQ ID NO: 40 - protein, 11 aa, synthetic construct
  REGION 1..11: note = R11
RRRRRRRRRRR

SEQ ID NO: 41 - protein, 10 aa, synthetic construct
  REGION 1..10: note = QSR8
QSRRRRRRRR

SEQ ID NO: 42 - protein, 14 aa, synthetic construct
  REGION 1..14: note = HIV-Tat derivative
GRKKRRQRRRPPQQ

SEQ ID NO: 43 - protein, 13 aa, Human immunodeficiency virus 1
GRKKRRQRRRPPQ

SEQ ID NO: 44 - protein, 18 aa, Human immunodeficiency virus 1
LGISYGRKKRRQRRRPPQ

SEQ ID NO: 45 - protein, 23 aa, Human immunodeficiency virus 1
FITKALGISYGRKKRRQRRRPPQ

SEQ ID NO: 46 - protein, 16 aa, Human immunodeficiency virus 1
FITKALGISYGRKKRR

SEQ ID NO: 47 - protein, 10 aa, Human immunodeficiency virus 1
GRKKRRQRRR

SEQ ID NO: 48 - protein, 9 aa, Human immunodeficiency virus 1
RKKRRQRRR

SEQ ID NO: 49 - protein, 8 aa, Human immunodeficiency virus 1
RKKRRQRR

SEQ ID NO: 50 - protein, 7 aa, Human immunodeficiency virus 1
RKKRRQR

SEQ ID NO: 51 - protein, 8 aa, Human immunodeficiency virus 1
KKRRQRRR

SEQ ID NO: 52 - protein, 7 aa, Human immunodeficiency virus 1
KRRQRRR

SEQ ID NO: 53 - protein, 9 aa, Human immunodeficiency virus 1
RKKRRQRRR

SEQ ID NO: 54 - protein, 9 aa, Human immunodeficiency virus 1
RRRQRRKKR

SEQ ID NO: 55 - protein, 9 aa, Human immunodeficiency virus 1
RRRQRRKKR

SEQ ID NO: 56 - protein, 9 aa, Human immunodeficiency virus 1
AKKRRQRRR

SEQ ID NO: 57 - protein, 9 aa, Human immunodeficiency virus 1
RAKRRQRRR

SEQ ID NO: 58 - protein, 9 aa, Human immunodeficiency virus 1
RKARRQRRR

SEQ ID NO: 59 - protein, 9 aa, Human immunodeficiency virus 1
RKKARQRRR

SEQ ID NO: 60 - protein, 9 aa, Human immunodeficiency virus 1
RKKRAQRRR

SEQ ID NO: 61 - protein, 9 aa, Human immunodeficiency virus 1
RKKRRARRR

SEQ ID NO: 62 - protein, 9 aa, Human immunodeficiency virus 1
RKKRRQARR

SEQ ID NO: 63 - protein, 9 aa, Human immunodeficiency virus 1
RKKRRQRAR

SEQ ID NO: 64 - protein, 9 aa, Human immunodeficiency virus 1
RKKRRQRRA

SEQ ID NO: 65 - protein, 13 aa, Human immunodeficiency virus 1
GRKKRRQRRPPQC

SEQ ID NO: 66 - protein, 12 aa, Human immunodeficiency virus 1
GRKKRRQRPPQC

SEQ ID NO: 67 - protein, 11 aa, Human immunodeficiency virus 1
GRKKRRQPPQC

SEQ ID NO: 68 - protein, 11 aa, Human immunodeficiency virus 1
GRKKRRQRRRC

SEQ ID NO: 69 - protein, 14 aa, Human immunodeficiency virus 1
GRKKRRQRARPPQC

SEQ ID NO: 70 - protein, 14 aa, Human immunodeficiency virus 1
GRKKRRQARAPPQC

SEQ ID NO: 71 - protein, 17 aa, Human immunodeficiency virus 1
TRQARRNRRRRWRERQR

SEQ ID NO: 72 - protein, 30 aa, Homo sapiens
GWTLNSAGYLLGPHAVGNHRSFSDKNGLTS

SEQ ID NO: 73 - protein, 14 aa, Vespula vulgaris
INLKALAALAKKIL

SEQ ID NO: 74 - protein, 16 aa, Drosophila melanogaster
RQIKIWFQNRRMKWKK

SEQ ID NO: 75 - protein, 16 aa, Drosophila melanogaster
RQIKIWFQNRRMKWKK

SEQ ID NO: 76 - protein, 16 aa, Drosophila melanogaster
KKWKMRRNQFWIKIQR

SEQ ID NO: 77 - protein, 16 aa, Drosophila melanogaster
RQIKIWFQNRRMKWKK

SEQ ID NO: 78 - protein, 16 aa, Drosophila melanogaster
RQIKIWFPNRRMKWKK

SEQ ID NO: 79 - protein, 16 aa, Drosophila melanogaster
RQPKIWFPNRRKPWKK

SEQ ID NO: 80 - protein, 16 aa, Drosophila melanogaster
RQIKIWFQNRRMKWKK

SEQ ID NO: 81 - protein, 15 aa, Drosophila melanogaster
RQIKIWFQNRRMKWK

SEQ ID NO: 82 - protein, 14 aa, Drosophila melanogaster
RQIKIWFQNRRMKW

SEQ ID NO: 83 - protein, 13 aa, Drosophila melanogaster
RQIKIWFQNRRMK

SEQ ID NO: 84 - protein, 12 aa, Drosophila melanogaster
RQIKIWFQNRRM

SEQ ID NO: 85 - protein, 11 aa, Drosophila melanogaster
RQIKIWFQNRR

SEQ ID NO: 86 - protein, 10 aa, Drosophila melanogaster
RQIKIWFQNR

SEQ ID NO: 87 - protein, 9 aa, Drosophila melanogaster
RQIKIWFQN

SEQ ID NO: 88 - protein, 8 aa, Drosophila melanogaster
RQIKIWFQ

SEQ ID NO: 89 - protein, 6 aa, Drosophila melanogaster
RQIKIW

SEQ ID NO: 90 - protein, 15 aa, Drosophila melanogaster
QIKIWFQNRRMKWKK

SEQ ID NO: 91 - protein, 14 aa, Drosophila melanogaster
IKIWFQNRRMKWKK

SEQ ID NO: 92 - protein, 13 aa, Drosophila melanogaster
KIWFQNRRMKWKK

SEQ ID NO: 93 - protein, 12 aa, Drosophila melanogaster
IWFQNRRMKWKK

SEQ ID NO: 94 - protein, 11 aa, Drosophila melanogaster
WFQNRRMKWKK

SEQ ID NO: 95 - protein, 10 aa, Drosophila melanogaster
FQNRRMKWKK

SEQ ID NO: 96 - protein, 9 aa, Drosophila melanogaster
QNRRMKWKK

SEQ ID NO: 97 - protein, 8 aa, Drosophila melanogaster
NRRMKWKK

SEQ ID NO: 98 - protein, 7 aa, Drosophila melanogaster
RRMKWKK

SEQ ID NO: 99 - protein, 6 aa, Drosophila melanogaster
RMKWKK

SEQ ID NO: 100 - protein, 16 aa, Drosophila melanogaster
AQIKIWFQNRRMKWKK

SEQ ID NO: 101 - protein, 16 aa, Drosophila melanogaster
RAIKIWFQNRRMKWKK

SEQ ID NO: 102 - protein, 16 aa, Drosophila melanogaster
RQAKIWFQNRRMKWKK

SEQ ID NO: 103 - protein, 16 aa, Drosophila melanogaster
RQIAIWFQNRRMKWKK

SEQ ID NO: 104 - protein, 16 aa, Drosophila melanogaster
RQIKAWFQNRRMKWKK

SEQ ID NO: 105 - protein, 16 aa, Drosophila melanogaster
RQIKIAFQNRRMKWKK

SEQ ID NO: 106 - protein, 16 aa, Drosophila melanogaster
RQIKIWAQNRRMKWKK

SEQ ID NO: 107 - protein, 16 aa, Drosophila melanogaster
RQIKIWFANRRMKWKK

SEQ ID NO: 108 - protein, 16 aa, Drosophila melanogaster
RQIKIWFQARRMKWKK

SEQ ID NO: 109 - protein, 16 aa, Drosophila melanogaster
RQIKIWFQNARMKWKK

SEQ ID NO: 110 - protein, 16 aa, Drosophila melanogaster
RQIKIWFQNRAMKWKK

SEQ ID NO: 111 - protein, 16 aa, Drosophila melanogaster
RQIKIWFQNRRAKWKK

SEQ ID NO: 112 - protein, 16 aa, Drosophila melanogaster
RQIKIWFQNRRMAWKK

SEQ ID NO: 113 - protein, 16 aa, Drosophila melanogaster
RQIKIWFQNRRMKAKK

SEQ ID NO: 114 - protein, 16 aa, Drosophila melanogaster
RQIKIWFQNRRMKWAK

SEQ ID NO: 115 - protein, 16 aa, Drosophila melanogaster
RQIKIWFQNRRMKWKA

SEQ ID NO: 116 - protein, 18 aa, Drosophila melanogaster
CRQIKIWFPNRRMKWKKC

SEQ ID NO: 117 - protein, 16 aa, Drosophila melanogaster
RQIKIWFPNRRMKWKK

SEQ ID NO: 118 - protein, 16 aa, Drosophila melanogaster
RQIKIWFQNRRMKWKK

SEQ ID NO: 119 - protein, 16 aa, Drosophila melanogaster
RQIKIFFQNRRMKFKK

SEQ ID NO: 120 - protein, 16 aa, Drosophila melanogaster
RQIRIWFQNRRMRWRR

SEQ ID NO: 121 - protein, 13 aa, Human immunodeficiency virus 1
GRKKRRQRRRPWQ

SEQ ID NO: 122 - protein, 13 aa, Human immunodeficiency virus 1
GRKKRRQRRRPWQ

SEQ ID NO: 123 - protein, 16 aa, Drosophila melanogaster
RQIRIWFQNRRMRWRR

SEQ ID NO: 124 - protein, 16 aa, Drosophila melanogaster
RRWRRWWRRWWRRWRR

SEQ ID NO: 125 - protein, 16 aa, Drosophila melanogaster
RQIKIWFQNMRRKWKK

SEQ ID NO: 126 - protein, 13 aa, Crotalus durissus
KMDCRWRWKCCKK

SEQ ID NO: 127 - protein, 12 aa, Crotalus durissus
MDCRWRWKCCKK

SEQ ID NO: 128 - protein, 11 aa, Crotalus durissus
DCRWRWKCCKK

SEQ ID NO: 129 - protein, 10 aa, Crotalus durissus
CRWRWKCCKK

SEQ ID NO: 130 - protein, 9 aa, Crotalus durissus
RWRWKCCKK

SEQ ID NO: 131 - protein, 12 aa, Crotalus durissus
KMDCRWRWKCKK

SEQ ID NO: 132 - protein, 11 aa, Crotalus durissus
KMDCRWRWKKK

SEQ ID NO: 133 - protein, 10 aa, Crotalus durissus
KMDRWRWKKK

SEQ ID NO: 134 - protein, 12 aa, Crotalus durissus
KDCRWRWKCCKK

SEQ ID NO: 135 - protein, 11 aa, Crotalus durissus
KCRWRWKCCKK

SEQ ID NO: 136 - protein, 10 aa, Crotalus durissus
KRWRWKCCKK

SEQ ID NO: 137 - protein, 12 aa, Crotalus durissus
  VARIANT 9: note = X can be any amino acid
MDCRWRWKXCKK

SEQ ID NO: 138 - protein, 11 aa, Crotalus durissus
  VARIANT 8: note = X can be any amino acid
DCRWRWKXCKK

SEQ ID NO: 139 - protein, 11 aa, Crotalus durissus
  VARIANT 9: note = X can be any amino acid
DCRWRWKCXKK

SEQ ID NO: 140 - protein, 10 aa, Crotalus durissus
  VARIANT 7: note = X can be any amino acid
CRWRWKXCKK

SEQ ID NO: 141 - protein, 10 aa, Crotalus durissus
  VARIANT 8: note = X can be any amino acid
CRWRWKCXKK

SEQ ID NO: 142 - protein, 9 aa, Crotalus durissus
  VARIANT 6: note = X can be any amino acid
RWRWKXCKK

SEQ ID NO: 143 - protein, 12 aa, Crotalus durissus
  VARIANT 9..10: note = X can be any amino acid
MDCRWRWKXXKK

SEQ ID NO: 144 - protein, 11 aa, Crotalus durissus
  VARIANT 8..9: note = X can be any amino acid
DCRWRWKXXKK

SEQ ID NO: 145 - protein, 10 aa, Crotalus durissus
  VARIANT 7..8: note = X can be any amino acid
CRWRWKXXKK

SEQ ID NO: 146 - protein, 9 aa, Crotalus durissus
  VARIANT 6..7: note = X can be any amino acid
RWRWKXXKK

SEQ ID NO: 147 - protein, 10 aa, Crotalus durissus
CRWRWKCSKK

SEQ ID NO: 148 - protein, 10 aa, Crotalus durissus
SRWRWKCCKK

SEQ ID NO: 149 - protein, 10 aa, Crotalus durissus
SRWRWKCSKK

SEQ ID NO: 150 - protein, 10 aa, Crotalus durissus
SRWRWKSCKK

SEQ ID NO: 151 - protein, 10 aa, Crotalus durissus
CRWRWKSSKK

SEQ ID NO: 152 - protein, 10 aa, Crotalus durissus
SRWRWKSSKK

SEQ ID NO: 153 - protein, 10 aa, Crotalus durissus
CRFRWKCCKK

SEQ ID NO: 154 - protein, 10 aa, Crotalus durissus
CRWRFKCCKK

SEQ ID NO: 155 - protein, 10 aa, Crotalus durissus
CRFRFKCCKK

SEQ ID NO: 156 - protein, 10 aa, Crotalus durissus
CRWRWKCCKK

SEQ ID NO: 157 - protein, 10 aa, Crotalus durissus
KCCKWRWRCK

SEQ ID NO: 158 - protein, 10 aa, Crotalus durissus
KCCKWRWRCK

SEQ ID NO: 159 - protein, 10 aa, Crotalus durissus
CRWRWKCCKK

SEQ ID NO: 160 - protein, 10 aa, Crotalus durissus
CRWRWKCCKK

SEQ ID NO: 161 - protein, 10 aa, Crotalus durissus
CRWRWKCCKK

SEQ ID NO: 162 - protein, 10 aa, Crotalus durissus
CRWRWKCCKK

SEQ ID NO: 163 - protein, 10 aa, Crotalus durissus
CRWRWKCCKK

SEQ ID NO: 164 - protein, 11 aa, Crotalus durissus
CRWRWKCGCKK

SEQ ID NO: 165 - protein, 14 aa, Crotalus durissus
KCGCRWRWKCGCKK

SEQ ID NO: 166 - protein, 8 aa, Crotalus durissus
CRWRWKCG

SEQ ID NO: 167 - protein, 13 aa, Crotalus durissus
  VARIANT 4: note = X can be any amino acid
KMDXRWRWKCCKK

SEQ ID NO: 168 - protein, 13 aa, Crotalus durissus
  VARIANT 4: note = X can be any amino acid
  VARIANT 10: note = X can be any amino acid
KMDXRWRWKXCKK

SEQ ID NO: 169 - protein, 13 aa, Crotalus durissus
  VARIANT 4: note = X can be any amino acid
  VARIANT 10..11: note = X can be any amino acid
KMDXRWRWKXXKK

SEQ ID NO: 170 - protein, 13 aa, Crotalus durissus
  VARIANT 4: note = X can be any amino acid
  VARIANT 11: note = X can be any amino acid
KMDXRWRWKCXKK

SEQ ID NO: 171 - protein, 12 aa, Crotalus durissus
  VARIANT 10: note = X can be any amino acid
MDCRWRWKCXKK

SEQ ID NO: 172 - protein, 13 aa, Crotalus durissus
KMDCRWRWKCSKK

SEQ ID NO: 173 - protein, 13 aa, Crotalus durissus
KMDCRWRWKSCKK

SEQ ID NO: 174 - protein, 13 aa, Crotalus durissus
KMDSRWRWKCCKK

SEQ ID NO: 175 - protein, 13 aa, Crotalus durissus
KMDCRWRWKSSKK

SEQ ID NO: 176 - protein, 13 aa, Crotalus durissus
KMDSRWRWKSSKK

SEQ ID NO: 177 - protein, 13 aa, Crotalus durissus
KMDSRWRWKSCKK

SEQ ID NO: 178 - protein, 13 aa, Crotalus durissus
KMDSRWRWKCSKK

SEQ ID NO: 179 - protein, 13 aa, Crotalus durissus
KMDCRWRPKCCKK

SEQ ID NO: 180 - protein, 13 aa, Crotalus durissus
KMDCRPRPKCCKK

SEQ ID NO: 181 - protein, 13 aa, Crotalus durissus
  VARIANT 4: note = X can be any amino acid
KMDXRPRPKCCKK

SEQ ID NO: 182 - protein, 13 aa, Crotalus durissus
  VARIANT 4: note = X can be any amino acid
  VARIANT 10: note = X can be any amino acid
KMDXRPRPKXCKK

SEQ ID NO: 183 - protein, 13 aa, Crotalus durissus
  VARIANT 4: note = X can be any amino acid
  VARIANT 11: note = X can be any amino acid
KMDXRPRPKCXKK

SEQ ID NO: 184 - protein, 13 aa, Crotalus durissus
  VARIANT 10: note = X can be any amino acid
KMDCRPRPKXCKK

SEQ ID NO: 185 - protein, 13 aa, Crotalus durissus
  VARIANT 11: note = X can be any amino acid
KMDCRPRPKCXKK

SEQ ID NO: 186 - protein, 16 aa, Drosophila melanogaster
RQIKIWFQNRRMKWKK

SEQ ID NO: 187 - protein, 9 aa, Human immunodeficiency virus 1
RKKRRQRRR

SEQ ID NO: 188 - protein, 9 aa, Human immunodeficiency virus 1
RRRQRRKKR

SEQ ID NO: 189 - protein, 16 aa, Homo sapiens
RKKRRRESRKKRRRES

SEQ ID NO: 190 - protein, 17 aa, Homo sapiens
GRPRESGKKRKRKRLKP

SEQ ID NO: 191 - protein, 15 aa, Homo sapiens
GKRKKKGKLGKKRDP

SEQ ID NO: 192 - protein, 17 aa, Homo sapiens
GKRKKKGKLGKKRPRSR

SEQ ID NO: 193 - protein, 18 aa, Homo sapiens
RKKRRRESRRARRSPRHL

SEQ ID NO: 194 - protein, 19 aa, Homo sapiens
SRRARRSPRESGKKRKRKR

SEQ ID NO: 195 - protein, 19 aa, Homo sapiens
VKRGLKLRHVRPRVTRMDV

SEQ ID NO: 196 - protein, 14 aa, Homo sapiens
SRRARRSPRHLGSG

SEQ ID NO: 197 - protein, 16 aa, Homo sapiens
LRRERQSRLRRERQSR

SEQ ID NO: 198 - protein, 22 aa, Homo sapiens
GAYDLRRRERQSRLRRRERQSR

SEQ ID NO: 199 - protein, 13 aa, Human immunodeficiency virus 1
GRKKRRQRRRPPQ

SEQ ID NO: 200 - protein, 5 aa, Homo sapiens
VPMLK

SEQ ID NO: 201 - protein, 5 aa, Homo sapiens
VPTLK

SEQ ID NO: 202 - protein, 5 aa, Homo sapiens
VPALR

SEQ ID NO: 203 - protein, 5 aa, Homo sapiens
VSALK

SEQ ID NO: 204 - protein, 5 aa, Homo sapiens
PMLKE

SEQ ID NO: 205 - protein, 5 aa, Homo sapiens
VPALK

SEQ ID NO: 206 - protein, 5 aa, Homo sapiens
VSLKK

SEQ ID NO: 207 - protein, 5 aa, Homo sapiens
VSGKK

SEQ ID NO: 208 - protein, 5 aa, Homo sapiens
KLPVM

SEQ ID NO: 209 - protein, 5 aa, Homo sapiens
IPMIK

SEQ ID NO: 210 - protein, 5 aa, Homo sapiens
KLGVM

SEQ ID NO: 211 - protein, 5 aa, Homo sapiens
KLPVT

SEQ ID NO: 212 - protein, 5 aa, Homo sapiens
VPMIK

SEQ ID NO: 213 - protein, 5 aa, Homo sapiens
IPALK

SEQ ID NO: 214 - protein, 5 aa, Homo sapiens
IPMLK

SEQ ID NO: 215 - protein, 5 aa, Homo sapiens
VPTLQ

SEQ ID NO: 216 - protein, 5 aa, Homo sapiens
QLPVM

SEQ ID NO: 217 - protein, 5 aa, Homo sapiens
ELPVM

SEQ ID NO: 218 - protein, 5 aa, Homo sapiens
VPTLE

SEQ ID NO: 219 - protein, 5 aa, Homo sapiens
VPTLK

SEQ ID NO: 220 - protein, 20 aa, Limulus polyphemus
AYRIKPTFRRLKWKYKGKFW

SEQ ID NO: 221 - protein, 20 aa, Limulus polyphemus
HARIKPTFRRLKWKYKGKFW

SEQ ID NO: 222 - protein, 20 aa, Limulus polyphemus
HYRIKPTARRLKWKYKGKFW

SEQ ID NO: 223 - protein, 20 aa, Limulus polyphemus
HYRIKPTFRRLAWKYKGKFW

SEQ ID NO: 224 - protein, 20 aa, Limulus polyphemus
HYRIKPTFRRLKWKYKGKFA

SEQ ID NO: 225 - protein, 21 aa, Mycobacterium tuberculosis
VNADIKATTVFGGKYVSLTTP

SEQ ID NO: 226 - protein, 22 aa, Mycobacterium tuberculosis
GKYVSLTTPKNPTKRRITPKDV

SEQ ID NO: 227 - protein, 22 aa, Mycobacterium tuberculosis
TKRRITPKDVIDVRSVTTEINT

SEQ ID NO: 228 - protein, 23 aa, Mycobacterium tuberculosis
RSVTTEINTLFQTLTSIAEKVDP

SEQ ID NO: 229 - protein, 26 aa, Mycobacterium tuberculosis
AEKVDPVKLNLTLSAAAEALTGLGDK

SEQ ID NO: 230 - protein, 34 aa, Mycobacterium tuberculosis
GLGDKFGESIVNANTVLDDLNSRMPQSRHDIQQL

SEQ ID NO: 231 - protein, 27 aa, Mycobacterium tuberculosis
GDVYADAAPDLFDFLDSSVTTARTINA

SEQ ID NO: 232 - protein, 32 aa, Mycobacterium tuberculosis
ARTINAQQAELDSALLAAAGFGNTTADVFDRG

SEQ ID NO: 233 - protein, 30 aa, Mycobacterium tuberculosis
ADVFDRGGPYLQRGVADLVPTATLLDTYSP

SEQ ID NO: 234 - protein, 27 aa, Mycobacterium tuberculosis
LDTYSPELFCTIRNFYDADRPDRGAAA

SEQ ID NO: 235 - protein, 22 aa, Mycobacterium tuberculosis
TKRRITPKDVIDVRSVTTEINT

SEQ ID NO: 236 - protein, 22 aa, Mycobacterium tuberculosis
TKRRITPDDVIDVRSVTTEINT

SEQ ID NO: 237 - protein, 22 aa, Mycobacterium tuberculosis
TKRRITPKKVIDVRSVTTEINT

SEQ ID NO: 238 - protein, 22 aa, Mycobacterium tuberculosis
TKRRITPKDVIDVRSVTTKINT

SEQ ID NO: 239 - protein, 13 aa, Mycobacterium tuberculosis
TKRRITPKDVIDV

SEQ ID NO: 240 - protein, 22 aa, Mycobacterium tuberculosis
TKRRITPKDVIDVESVTTEINT

SEQ ID NO: 241 - protein, 22 aa, Mycobacterium tuberculosis
TARRITPKDVIDVRSVTTEINT

SEQ ID NO: 242 - protein, 23 aa, Mycobacterium tuberculosis
TKAARITPKDVIDVRSVTTEINT

SEQ ID NO: 243 - protein, 28 aa, Mycobacterium tuberculosis
HHHHHHTKRRITPKDVIDVRSVTTEINT

SEQ ID NO: 244 - protein, 12 aa, Human immunodeficiency virus 1
YGRKKKRRQRRR

SEQ ID NO: 245 - protein, 18 aa, Mus musculus
LLIILRRRIRKQAHAHSK

SEQ ID NO: 246 - protein, 18 aa, Mus musculus
ALIILRRRIRKQAHAHSK

SEQ ID NO: 247 - protein, 18 aa, Mus musculus
LAIILRRRIRKQAHAHSK

SEQ ID NO: 248 - protein, 18 aa, Mus musculus
LLAILRRRIRKQAHAHSK

SEQ ID NO: 249 - protein, 18 aa, Mus musculus
LLIALRRRIRKQAHAHSK

SEQ ID NO: 250 - protein, 18 aa, Mus musculus
LLIIARRRIRKQAHAHSK

SEQ ID NO: 251 - protein, 18 aa, Mus musculus
LLIILARRIRKQAHAHSK

SEQ ID NO: 252 - protein, 18 aa, Mus musculus
LLIILRARIRKQAHAHSK

SEQ ID NO: 253 - protein, 18 aa, Mus musculus
LLIILRRAIRKQAHAHSK

SEQ ID NO: 254 - protein, 18 aa, Mus musculus
LLIILRRRARKQAHAHSK

SEQ ID NO: 255 - protein, 19 aa, Mus musculus
LLIILRRRIARKQAHAHSK

SEQ ID NO: 256 - protein, 18 aa, Mus musculus
LLIILRRRIRAQAHAHSK

SEQ ID NO: 257 - protein, 18 aa, Mus musculus
LLIILRRRIRKAAHAHSK

SEQ ID NO: 258 - protein, 18 aa, Mus musculus
LLIILRRRIRKQAHAHSK

SEQ ID NO: 259 - protein, 18 aa, Mus musculus
LLIILRRRIRKQAAAHSK

SEQ ID NO: 260 - protein, 18 aa, Mus musculus
LLIILRRRIRKQAHAHSK

SEQ ID NO: 261 - protein, 18 aa, Mus musculus
LLIILRRRIRKQAHAASK

SEQ ID NO: 262 - protein, 18 aa, Mus musculus
LLIILRRRIRKQAHAHAK

SEQ ID NO: 263 - protein, 18 aa, Mus musculus
LLIILRRRIRKQAHAHSA

SEQ ID NO: 264 - protein, 18 aa, Mus musculus
KSHAHAQKRIRRRLIILL

SEQ ID NO: 265 - protein, 18 aa, Mus musculus
LLIILRRRIRKQAHAHSK

SEQ ID NO: 266 - protein, 7 aa, Bos primigenius
RRIRPRP

SEQ ID NO: 267 - protein, 15 aa, Bos primigenius
RRIRPRPPRLPRPRP

SEQ ID NO: 268 - protein, 24 aa, Bos primigenius
RRIRPRPPRLPRPRPRPLPFPRPG

SEQ ID NO: 269 - protein, 17 aa, Bos primigenius
RRIRPRPPRLPRPRPRP

SEQ ID NO: 270 - protein, 20 aa, Bos primigenius
PRPPRLPRPRPRPLPFPRPG

SEQ ID NO: 271 - protein, 18 aa, Bos primigenius
PPRLPRPRPRPLPFPRPG

SEQ ID NO: 272 - protein, 16 aa, Bos primigenius
RLPRPRPRPLPFPRPG

SEQ ID NO: 273 - protein, 14 aa, Bos primigenius
PRPRPRPLPFPRPG

SEQ ID NO: 274 - protein, 12 aa, Bos primigenius
PRPRPLPFPRPG

SEQ ID NO: 275 - protein, 10 aa, Bos primigenius
PRPLPFPRPG

SEQ ID NO: 276 - protein, 9 aa, Human immunodeficiency virus 1
RKKRRQRRR

SEQ ID NO: 277 - protein, 27 aa, African swine fever virus
RQGAARVTSWLGRQLRIAGKRLEGRSK

SEQ ID NO: 278 - protein, 22 aa, African swine fever virus
RVTSWLGRQLRIAGKRLEGRSK

SEQ ID NO: 279 - protein, 16 aa, African swine fever virus
GRQLRIAGKRLEGRSK

SEQ ID NO: 280 - protein, 23 aa, African swine fever virus
RRVTSWLGRQLRIAGKRLEGRSK

SEQ ID NO: 281 - protein, 22 aa, African swine fever virus
RVRSWLGRQLRIAGKRLEGRSK

SEQ ID NO: 282 - protein, 16 aa, African swine fever virus
GRQLRIAGKRLRGRSK

SEQ ID NO: 283 - protein, 16 aa, African swine fever virus
GRQLRIAGRRLRGRSR

SEQ ID NO: 284 - protein, 16 aa, African swine fever virus
GRQLRRAGRRLRGRSR

SEQ ID NO: 285 - protein, 16 aa, African swine fever virus
GRQLRIAGRRLRRRSR

SEQ ID NO: 286 - protein, 16 aa, African swine fever virus
GRQLRRAGRRLRRRSR

SEQ ID NO: 287 - protein, 15 aa, African swine fever virus
RQLRIAGRRLRGRSR

SEQ ID NO: 288 - protein, 16 aa, African swine fever virus
RSRGRLRRGAIRLQRG

SEQ ID NO: 289 - protein, 29 aa, Aspergillus restrictus
KLIKGRTPIKFGKADCDRPPKHSQNGMGK

SEQ ID NO: 290 - protein, 27 aa, Aspergillus restrictus
KLIKGRTPIKFGKADCDRPPKHSQNGM

SEQ ID NO: 291 - protein, 27 aa, Aspergillus restrictus
KLIKGRTPIKFGKADCDRPPKHSQNGK

SEQ ID NO: 292 - protein, 26 aa, Aspergillus restrictus
KGRTPIKFGKADCDRPPKHSQNGMGK

SEQ ID NO: 293 - protein, 25 aa, Aspergillus restrictus
KLIKGRTPIKFGKADCDRPPKHSGK

SEQ ID NO: 294 - protein, 25 aa, Aspergillus restrictus
KLIKGRTPIKFGKARCRRPPKHSGK

SEQ ID NO: 295 - protein, 13 aa, Aspergillus restrictus
KLIKGRTPIKFGK

SEQ ID NO: 296 - protein, 37 aa, Homo sapiens
KRIPNKKPGKKTTTKPTKKPTIKTTKKDLKPQTTKPK

SEQ ID NO: 297 - protein, 30 aa, Homo sapiens
KRIPNKKPGKKTTTKPTKKPTIKTTKKDLK

SEQ ID NO: 298 - protein, 27 aa, Homo sapiens
KRIPNKKPGKKTTTKPTKKPTIKTTKK

SEQ ID NO: 299 - protein, 23 aa, Homo sapiens
KRIPNKKPGKKTTTKPTKKPTIK

SEQ ID NO: 300 - protein, 19 aa, Homo sapiens
KRIPNKKPGKKTTTKPTKK

SEQ ID NO: 301 - protein, 12 aa, Homo sapiens
KRIPNKKPGKKT

SEQ ID NO: 302 - protein, 11 aa, Homo sapiens
KRIPNKKPGKK

SEQ ID NO: 303 - protein, 10 aa, Homo sapiens
KRIPNKKPKK

SEQ ID NO: 304 - protein, 10 aa, Homo sapiens
RRIPNRRPRR

SEQ ID NO: 305 - protein, 22 aa, Homo sapiens
KKPGKKTTTKPTKKPTIKTTKK

SEQ ID NO: 306 - protein, 14 aa, Homo sapiens
KKPGKKTTTKPTKK

SEQ ID NO: 307 - protein, 10 aa, Homo sapiens
KKPTIKTTKK

SEQ ID NO: 308 - protein, 10 aa, Homo sapiens
KKTTTKPTKK

SEQ ID NO: 309 - protein, 15 aa, Homo sapiens
KSICKTIPSNKPKKK

SEQ ID NO: 310 - protein, 11 aa, Homo sapiens
KTIPSNKPKKK

SEQ ID NO: 311 - protein, 12 aa, Homo sapiens
KPRSKNPPKKPK

SEQ ID NO: 312 - protein, 22 aa, Human immunodeficiency virus 1
DRRRRGSRPSGAERRRRRAAAA

SEQ ID NO: 313 - protein, 17 aa, Human immunodeficiency virus 1
DRRRRGSRPSGAERRRR

SEQ ID NO: 314 - protein, 15 aa, Bacteriophage lambda
QTRRRERRAEKQAQW

SEQ ID NO: 315 - protein, 9 aa, Bacteriophage lambda
RRRERRAEK

SEQ ID NO: 316 - protein, 11 aa, Flock House virus
NRARRNRRRVR

SEQ ID NO: 317 - protein, 10 aa, Flock House virus
RTRRNRRRVR

SEQ ID NO: 318 - protein, 8 aa, Alphavirus
RNRSRHRR

SEQ ID NO: 319 - protein, 9 aa, Alphavirus
KCPSRRPKR

SEQ ID NO: 320 - protein, 16 aa, Drosophila melanogaster
KRPAAIKKAGQAKKKK

SEQ ID NO: 321 - protein, 12 aa, Kaposi's sarcoma-associated herpesvirus
TRRSKRRSHRKF

SEQ ID NO: 322 - protein, 17 aa, Bufo bufo
RAGLQFPVGRVHRLLRK

SEQ ID NO: 323 - protein, 22 aa, Homo sapiens
MVRRFLVTLRIRRACGPPRVRV

SEQ ID NO: 324 - protein, 22 aa, Homo sapiens
FVTRGCPRRLVARLIRVMVPRR

SEQ ID NO: 325 - protein, 13 aa, Homo sapiens
VRRFLVTLRIRRA

SEQ ID NO: 326 - protein, 13 aa, Homo sapiens
RVRILARFLRTRV

SEQ ID NO: 327 - protein, 13 aa, Homo sapiens
RVRVFVVHIPRLT

SEQ ID NO: 328 - protein, 13 aa, Homo sapiens
VIRVHFRLPVRTV

SEQ ID NO: 329 - protein, 37 aa, Homo sapiens
MVRRFLVTLRIRRACGPPRVRVFVVHIPRLTGEWAAP

SEQ ID NO: 330 - protein, 37 aa, Homo sapiens
FRVPLRIRPCVVAPRLVMVRHTFGRIARWVAGPLETR

SEQ ID NO: 331 - protein, 25 aa, Homo sapiens
GTKMIFVGIKKKEERADLIAYLKKA

SEQ ID NO: 332 - protein, 16 aa, Homo sapiens
KKKEERADLIAYLKKA

SEQ ID NO: 333 - protein, 14 aa, Homo sapiens
KMIFVGIKKKEERA

SEQ ID NO: 334 - protein, 10 aa, Homo sapiens
KMIFVGIKKK

SEQ ID NO: 335 - protein, 10 aa, Homo sapiens
EKGKKIFIMK

SEQ ID NO: 336 - protein, 9 aa, Homo sapiens
KGKKIFIMK

SEQ ID NO: 337 - protein, 17 aa, Flock House virus
RRRRNRTRRNRRRVRGC

SEQ ID NO: 338 - protein, 15 aa, Human T-cell lymphotropic virus type 2
TRRQRTRRARRNRGC

SEQ ID NO: 339 - protein, 21 aa, Brome mosaic virus
KMTRAQRRAAARRNRWTARGC

SEQ ID NO: 340 - protein, 21 aa, Cowpea chlorotic mottle virus
KLTRAQRRAAARKNKRNTRGC

SEQ ID NO: 341 - protein, 19 aa, Bacteriophage P22
NAKTRRHERRRKLAIERGC

SEQ ID NO: 342 - protein, 24 aa, Bacteriophage lambda
MDAQTRRRERRAEKQAQWKAANGC

SEQ ID NO: 343 - protein, 20 aa, Bacteriophage phi-21
TAKTRYKARRAELIAERRGC

SEQ ID NO: 344 - protein, 14 aa, Homo sapiens
SQMTRQARRLYBGC

SEQ ID NO: 345 - protein, 28 aa, Homo sapiens
KRRIRRERNKMAAAKSRNRRRELTDTGC

SEQ ID NO: 346 - protein, 30 aa, Homo sapiens
RIKAERKRMRNRIAASKSRKRKLERIARGC

SEQ ID NO: 347 - protein, 24 aa, Saccharomyces cerevisiae
KRARNTEAARRSRARKLQRMKQGC

SEQ ID NO: 348 - protein, 22 aa, Homo sapiens
KCFQWQRNMRKVRGPPVSCIKR

SEQ ID NO: 349 - protein, 19 aa, Homo sapiens
KCFQWQRNMRKVRGPPVSC

SEQ ID NO: 350 - protein, 22 aa, Homo sapiens
KCFQWQRNMRKVRGPPVSSIKR

SEQ ID NO: 351 - protein, 13 aa, Homo sapiens
KCFQWQRNMRKVR

SEQ ID NO: 352 - protein, 16 aa, Homo sapiens
FQWQRNMRKVRGPPVS

SEQ ID NO: 353 - protein, 19 aa, Homo sapiens
QWQRNMRKVRGPPVSCIKR

SEQ ID NO: 354 - protein, 10 aa, Homo sapiens
QWQRNMRKVR

SEQ ID NO: 355 - protein, 16 aa, Drosophila melanogaster
RQIKIWFQNRRMKWKK

SEQ ID NO: 356 - protein, 22 aa, Rattus rattus
KCFMWQEMLNKAGVPKLRCARK

SEQ ID NO: 357 - protein, 20 aa, Homo sapiens
GLWRALWRLLRSLWRLLWRA

SEQ ID NO: 358 - protein, 20 aa, Homo sapiens
GLWWRLWWRLRSWFRLWFRA

SEQ ID NO: 359 - protein, 34 aa, Human alphaherpesvirus 1
DAATATRGRSAASRPTQRPRAPARSASRPRRPVE

SEQ ID NO: 360 - protein, 34 aa, Homo sapiens
AKVKDEPQRRSARLSAKPAPPKPEPKPKKAPAKK

SEQ ID NO: 361 - protein, 34 aa, Homo sapiens; identical to SEQ ID NO: 360

SEQ ID NO: 362 - protein, 12 aa, Hepatitis B virus
PLSSIFSRIGDP

SEQ ID NO: 363 - protein, 12 aa, Hepatitis B virus
PSSSSSSRIGDP

SEQ ID NO: 364 - protein, 18 aa, Zea mays
VRLPPPVRLPPPVRLPPP

SEQ ID NO: 365 - protein, 18 aa, Zea mays
VELPPPVELPPPVELPPP

SEQ ID NO: 366 - protein, 28 aa, Homo sapiens
ALWMTLLKKVLKAAAKAALNAVLVGANA

SEQ ID NO: 367 - protein, 13 aa, Homo sapiens
ALWKTLLKKVLKA

SEQ ID NO: 368 - protein, 10 aa, Human immunodeficiency virus 1
RQARRNRRRC

SEQ ID NO: 369 - protein, 14 aa, Human immunodeficiency virus 1
GRKKRRQRRRPPQC

SEQ ID NO: 370 - protein, 8 aa, Homo sapiens
GRKRKKRT

SEQ ID NO: 371 - protein, 22 aa, Homo sapiens
MVTVLFRRLRIRRACGPPRVRV

SEQ ID NO: 372 - protein, 16 aa, Drosophila melanogaster
RQIKIWFQNRRMKWKK

SEQ ID NO: 373 - protein, 10 aa, Homo sapiens
VQRKRQKLMP

SEQ ID NO: 374 - protein, 8 aa, Homo sapiens
SKKKKTKV

SEQ ID NO: 375 - protein, 8 aa, Homo sapiens
GRKRKKRT

SEQ ID NO: 376 - protein, 11 aa, Homo sapiens
GKKKKRKREKL

SEQ ID NO: 377 - protein, 7 aa, Homo sapiens
PKKKRKV

SEQ ID NO: 378 - protein, 8 aa, Caenorhabditis elegans
ERKKRRRE

SEQ ID NO: 379 - protein, 7 aa, Homo sapiens
FKKFRKF

SEQ ID NO: 380 - protein, 24 aa, Homo sapiens
LGTYTQDFNKFHTFPQTAIGVGAP

SEQ ID NO: 381 - protein, 24 aa, Homo sapiens
LGTYTQDFNKFHTFPQTAIGVGAP

SEQ ID NO: 382 - protein, 21 aa, Homo sapiens
YTQDFNKFHTFPQTAIGVGAP

SEQ ID NO: 383 - protein, 18 aa, Homo sapiens
DFNKFHTFPQTAIGVGAP

SEQ ID NO: 384 - protein, 15 aa, Homo sapiens
KFHTFPQTAIGVGAP

SEQ ID NO: 385 - protein, 12 aa, Homo sapiens
TFPQTAIGVGAP

SEQ ID NO: 386 - protein, 13 aa, Human immunodeficiency virus 1
GYGRKKRRQRRRG

SEQ ID NO: 387 - protein, 13 aa, Human immunodeficiency virus 1
GYGRKKRRQRRRG

SEQ ID NO: 388 - protein, 13 aa, Human immunodeficiency virus 1
GYGRKKRRQRRRG

SEQ ID NO: 389 - protein, 16 aa, Drosophila melanogaster
RQIKIWFQNRRMKWKK

SEQ ID NO: 390 - protein, 16 aa, Drosophila melanogaster
RQIKIWFQNRRMKWKK

SEQ ID NO: 391 - protein, 16 aa, Drosophila melanogaster
RQIKIWFQNRRMKWKK

SEQ ID NO: 392 - protein, 15 aa, Homo sapiens
FLGKKFKKYFLQLLK

SEQ ID NO: 393 - protein, 23 aa, Homo sapiens
FLIFIRVICIVIAKLKANLMCKT

SEQ ID NO: 394 - protein, 17 aa, Homo sapiens
KKAAQIRSQVMTHLRVI

SEQ ID NO: 395 - protein, 16 aa, Homo sapiens
YIVLRRRRKRVNTKRS

SEQ ID NO: 396 - protein, 11 aa, Homo sapiens
RRKLSQQKEKK

SEQ ID NO: 397 - protein, 15 aa, Homo sapiens
VQAILRRNWNQYKIQ

SEQ ID NO: 398 - protein, 19 aa, Homo sapiens
KLPCRSNTFLNIFRRKKPG

SEQ ID NO: 399 - protein, 16 aa, Homo sapiens
KKICTRKPRFMSAWAQ

SEQ ID NO: 400 - protein, 16 aa, Drosophila melanogaster
RQIKIWFQNRRMKWKK

SEQ ID NO: 401 - protein, 18 aa, Sus scrofa
RGGRLSYSRRRFSTSTGR

SEQ ID NO: 402 - protein, 18 aa, Sus scrofa
RGGRLSYSRRRFSTSTGR

SEQ ID NO: 403 - protein, 10 aa, Sus scrofa
RRLSYSRRRF

SEQ ID NO: 404 - protein, 10 aa, Sus scrofa
RRLSYSRRRF

SEQ ID NO: 405 - protein, 17 aa, Sus scrofa
RGGRLAYLRRRWAVLGR

SEQ ID NO: 406 - protein, 16 aa, Drosophila melanogaster
RQIKIWFQNRRMKWKK

SEQ ID NO: 407 - protein, 28 aa, Mus musculus
MANLGYWLLALFVTMWTDVGLCKKRPKP

SEQ ID NO: 408 - protein, 28 aa, Homo sapiens
MANLGCWMLVLFVATWSDLGLCKKRPKP

SEQ ID NO: 409 - protein, 30 aa, Bos primigenius
MVKSKIGSWILVLFVAMWSDVGLCKKRPKP

SEQ ID NO: 410 - protein, 38 aa, Homo sapiens
LLGDFFRKSKEKIGKEFKRIVQRIKDFLRNLVPRTESC

SEQ ID NO: 411 - protein, 16 aa, Drosophila melanogaster
RQIKIWFQNRRMKWKK

SEQ ID NO: 412 - protein, 16 aa, Rattus rattus
RVIRVWFQNKRCKDKK

SEQ ID NO: 413 - protein, 23 aa, Xenopus laevis
GIGKFLHSAKKWGKAFVGQIMNC

SEQ ID NO: 414 - protein, 24 aa, Bufo bufo
TRSSRAGLQWPVGRVHRLLRKGGC

SEQ ID NO: 415 - protein, 11 aa, Human immunodeficiency virus 1
YGRKKRRQRRR

SEQ ID NO: 416 - protein, 16 aa, Homo sapiens
RHIKIWFQNRRMKWKK

SEQ ID NO: 417 - protein, 9 aa, Human immunodeficiency virus 1
RKKRRQRRR

SEQ ID NO: 418 - protein, 16 aa, Drosophila melanogaster
RQIKIWFQNRRMKWKK

SEQ ID NO: 419 - protein, 60 aa, Drosophila melanogaster
SKRTRQTYTRYQTLELEKEFHFNRYITRRRRIDIANALSLSERQIKIWFQNRRMKSKKDR

SEQ ID NO: 420 - protein, 61 aa, Drosophila melanogaster
EKRPRTAFSSEQLARLKREFNENRYLTTERRRQQLSSELGLNEAQIKIWFQNKRAKIKKST

SEQ ID NO: 421 - protein, 13 aa, Human immunodeficiency virus 1
GRRRRRRRRRPPQ

SEQ ID NO: 422 - protein, 10 aa, Homo sapiens
MLLLTRRRST

SEQ ID NO: 423 - protein, 16 aa, Homo sapiens
GLRKRLRKFRNKIKEK

SEQ ID NO: 424 - protein, 6 aa, Zea mays
VRLPPP

SEQ ID NO: 425 - protein, 12 aa, Zea mays
VRLPPPVRLPPP

SEQ ID NO: 426 - protein, 18 aa, Zea mays
VRLPPPVRLPPPVRLPPP

SEQ ID NO: 427 - protein, 6 aa, Zea mays
VHLPPP

SEQ ID NO: 428 - protein, 12 aa, Zea mays
VHLPPPVHLPPP

SEQ ID NO: 429 - protein, 18 aa, Zea mays
VHLPPPVHLPPPVHLPPP

SEQ ID NO: 430 - protein, 6 aa, Zea mays
VKLPPP

SEQ ID NO: 431 - protein, 12 aa, Zea mays
VKLPPPVKLPPP

SEQ ID NO: 432 - protein, 18 aa, Zea mays
VKLPPPVKLPPPVKLPPP

SEQ ID NO: 433 - protein, 16 aa, Drosophila melanogaster
RQIKIWFQNRRMKWKK

SEQ ID NO: 434 - protein, 16 aa, Drosophila melanogaster
RQIKIFFQNRRMKWKK

SEQ ID NO: 435 - protein, 21 aa, Flock House virus
ASMWERVKSIIKSSLAAASNI

SEQ ID NO: 436 - protein, 21 aa, Flock House virus
ASMWERVKSIIKSSLAAASNI

SEQ ID NO: 437 - protein, 16 aa, Homo sapiens
CSIPPEVKFNPFVYLI

SEQ ID NO: 438 - protein, 16 aa, Homo sapiens
CSIPPEVKFNPFVYLI

SEQ ID NO: 439 - protein, 6 aa, Homo sapiens
PFVYLI

SEQ ID NO: 440 - protein, 8 aa, Homo sapiens
NKPILVFY

SEQ ID NO: 441 - protein, 14 aa, Crotalus durissus
YKQCHKKGGKKGSG

SEQ ID NO: 442 - protein, 15 aa, Crotalus durissus
  VARIANT 10: note = X can be any amino acid
YKQCHKKGGXKKGSG

SEQ ID NO: 443 - protein, 14 aa, Crotalus durissus
GSGKKGGKKHCQKY

SEQ ID NO: 444 - protein, 14 aa, Crotalus durissus
GSGKKGGKKICQKY

SEQ ID NO: 445 - protein, 23 aa, Drosophila melanogaster
LIRLWSHLIHIWFQNRRLKWKKK

SEQ ID NO: 446 - protein, 16 aa, Homo sapiens
AAVALLPAVLLALLAP

SEQ ID NO: 447 - protein, 23 aa, Homo sapiens
CGAYDLRRRERQSRLRRRERQSR

SEQ ID NO: 448 - protein, 17 aa, Homo sapiens
RKKRRRESRKKRRRESC

SEQ ID NO: 449 - protein, 19 aa, Homo sapiens
CVKRGLKLRHVRPRVTRDV

SEQ ID NO: 450 - protein, 17 aa, Drosophila melanogaster
CRQIKIWFQNRRMKWKK

SEQ ID NO: 451 - protein, 15 aa, Homo sapiens
PPKKSAQCLRYKKPE

SEQ ID NO: 452 - protein, 15 aa, Homo sapiens
DPVDTPNPTRRKPGK

SEQ ID NO: 453 - protein, 13 aa, Homo sapiens
KRVSRNKSEKKRR

SEQ ID NO: 454 - protein, 16 aa, Homo sapiens
GRRHHCRSKAKRSRHH

SEQ ID NO: 455 - protein, 16 aa, Homo sapiens
SARHHCRSKAKRSRHH

SEQ ID NO: 456 - protein, 16 aa, Homo sapiens
SRAHHCRSKAKRSRHH

SEQ ID NO: 457 - protein, 16 aa, Homo sapiens
SRRAHCRSKAKRSRHH

SEQ ID NO: 458 - protein, 16 aa, Homo sapiens
SRRHACRSKAKRSRHH

SEQ ID NO: 459 - protein, 16 aa, Homo sapiens
SRRHHARSKAKRSRHH

SEQ ID NO: 460 - protein, 16 aa, Homo sapiens
SRRHHCRAKAKRSRHH

SEQ ID NO: 461 - protein, 16 aa, Homo sapiens
SRRHHCRSAAKRSRHH

SEQ ID NO: 462 - protein, 16 aa, Homo sapiens
SRRHHCRSKAARSRHH

SEQ ID NO: 463 - protein, 16 aa, Homo sapiens
SRRHHCRSKAKASRHH

SEQ ID NO: 464 - protein, 16 aa, Homo sapiens
SRRHHCRSKAKRARHH

SEQ ID NO: 465 - protein, 16 aa, Homo sapiens
SRRHHCRSKAKRSAHH

SEQ ID NO: 466 - protein, 13 aa, Homo sapiens
RRHHCRSKAKRSR

SEQ ID NO: 467 - protein, 12 aa, Homo sapiens
GRKGKHKRKKLP

SEQ ID NO: 468 - protein, 18 aa, Homo sapiens
GKRVAKRKLIEQNRERRR

SEQ ID NO: 469 - protein, 18 aa, Homo sapiens
GRKLKKKKNEKEDKRPRT

SEQ ID NO: 470 - protein, 17 aa, Homo sapiens
GKKTNLFSALIKKKKTA

SEQ ID NO: 471 - protein, 18 aa, Homo sapiens
GRRERNKMAAAKCRNRRR

SEQ ID NO: 472 - protein, 18 aa, Homo sapiens
GKRARNTEAARRSRARKL

SEQ ID NO: 473 - protein, 14 aa, Homo sapiens
GRRRRATAKYRTAH

SEQ ID NO: 474 - protein, 15 aa, Homo sapiens
GKRRRRATAKYRSAH

SEQ ID NO: 475 - protein, 12 aa, Homo sapiens
GRRRRKRLSHRT

SEQ ID NO: 476 - protein, 10 aa, Homo sapiens
GRRRRRERNK

SEQ ID NO: 477 - protein, 16 aa, Homo sapiens
GKHRHERGHHRDRRER

SEQ ID NO: 478 - protein, 17 aa, Homo sapiens
GKKKRKLSNRESAKRSR

SEQ ID NO: 479 - protein, 10 aa, Homo sapiens
MIIYRDLISH

SEQ ID NO: 480 - protein, 9 aa, Homo sapiens
MIIYRDLIS

SEQ ID NO: 481 - protein, 8 aa, Homo sapiens
MIIYRDLI

SEQ ID NO: 482 - protein, 9 aa, Homo sapiens
IIYRDLISH

SEQ ID NO: 483 - protein, 7 aa, Homo sapiens
MIIYRDL

SEQ ID NO: 484 - protein, 6 aa, Homo sapiens
MIIYRD

SEQ ID NO: 485 - protein, 8 aa, Homo sapiens
IYRDLISH

SEQ ID NO: 486 - protein, 9 aa, Homo sapiens
AIIYRDLIS

SEQ ID NO: 487 - protein, 9 aa, Homo sapiens
MAIYRDLIS

SEQ ID NO: 488 - protein, 9 aa, Homo sapiens
MIAYRDLIS

SEQ ID NO: 489 - protein, 9 aa, Homo sapiens
MIIARDLIS

SEQ ID NO: 490 - protein, 9 aa, Homo sapiens
MIIYADLIS

SEQ ID NO: 491 - protein, 9 aa, Homo sapiens
MIIYRALIS

SEQ ID NO: 492 - protein, 9 aa, Homo sapiens
MIIYRDAIS

SEQ ID NO: 493 - protein, 9 aa, Homo sapiens
MIIYRDLAS

SEQ ID NO: 494 - protein, 9 aa, Homo sapiens
MIIYRDLIA

SEQ ID NO: 495 - protein, 11 aa, Homo sapiens
MIIYRDLISKK

SEQ ID NO: 496 - protein, 10 aa, Homo sapiens
MIIYRDKKSH

SEQ ID NO: 497 - protein, 10 aa, Homo sapiens
MIIFRDLISH

SEQ ID NO: 498 - protein, 10 aa, Homo sapiens
MIISRDLISH

SEQ ID NO: 499 - protein, 10 aa, Homo sapiens
QIISRDLISH

SEQ ID NO: 500 - protein, 10 aa, Homo sapiens
CIISRDLISH

SEQ ID NO: 501 - protein, 12 aa, Homo sapiens
MIIYRALISHKK

SEQ ID NO: 502 - protein, 12 aa, Homo sapiens
MIIYRIAASHKK

SEQ ID NO: 503 - protein, 10 aa, Homo sapiens
MIIRRDLISE

SEQ ID NO: 504 - protein, 10 aa, Homo sapiens
MIIYRAEISH

SEQ ID NO: 505 - protein, 10 aa, Homo sapiens
MIIYARRAEE

SEQ ID NO: 506 - protein, 12 aa, Homo sapiens
MIIFRIAASHKK

SEQ ID NO: 507 - protein, 12 aa, Homo sapiens
MIIFRALISHKK

SEQ ID NO: 508 - protein, 12 aa, Homo sapiens
MIIFRAAASHKK

SEQ ID NO: 509 - protein, 12 aa, Homo sapiens
FIIFRIAASHKK

SEQ ID NO: 510 - protein, 12 aa, Homo sapiens
LIIFRIAASHKK

SEQ ID NO: 511 - protein, 12 aa, Homo sapiens
WIIFRIAASHKK

SEQ ID NO: 512 - protein, 12 aa, Homo sapiens
WIIFRAAASHKK

SEQ ID NO: 513 - protein, 12 aa, Homo sapiens
WIIFRALISHKK

SEQ ID NO: 514 - protein, 12 aa, Homo sapiens
MIIFRIAAYHKK

SEQ ID NO: 515 - protein, 12 aa, Homo sapiens
WIIFRIAAYHKK

SEQ ID NO: 516 - protein, 12 aa, Homo sapiens
MIIFRIAATHKK

SEQ ID NO: 517 - protein, 12 aa, Homo sapiens
WIIFRIAATHKK

SEQ ID NO: 518 - protein, 12 aa, Homo sapiens
MIIFKIAASHKK

SEQ ID NO: 519 - protein, 12 aa, Homo sapiens
WIIFKIAASHKK

SEQ ID NO: 520 - protein, 12 aa, Homo sapiens
MIIFAIAASHKK

SEQ ID NO: 521 - protein, 12 aa, Homo sapiens
LIIFRILISHKK

SEQ ID NO: 522 - protein, 12 aa, Homo sapiens
MIIFRILISHKK

SEQ ID NO: 523 - protein, 12 aa, Homo sapiens
LIIFRILISHRR

SEQ ID NO: 524 - protein, 12 aa, Homo sapiens
LIIFRILISHHH

SEQ ID NO: 525 - protein, 11 aa, Homo sapiens
LIIFRILISHK

SEQ ID NO: 526 - protein, 11 aa, Homo sapiens
LIIFRILISHR

SEQ ID NO: 527 - protein, 10 aa, Homo sapiens
LIIFRILISH

SEQ ID NO: 528 - protein, 12 aa, Homo sapiens
LIIFAIAASHKK

SEQ ID NO: 529 - protein, 12 aa, Homo sapiens
LIIFAILISHKK

SEQ ID NO: 530 - protein, 16 aa, Drosophila melanogaster
RQIKIWFQNRRMKWKK

SEQ ID NO: 531 - protein, 16 aa, Homo sapiens
AAVALLPAVLLALLAK

SEQ ID NO: 532 - protein, 26 aa, Drosophila melanogaster
AHALCLTERQIKIWFQNRRMKWKKEN

SEQ ID NO: 533 - protein, 26 aa, Drosophila melanogaster
AHALCPPERQIKIWFQNRRMKWKKEN

SEQ ID NO: 534 - protein, 26 aa, Drosophila melanogaster
AYALCLTERQIKIWFANRRMKWKKEN

SEQ ID NO: 535 - protein, 34 aa, Momordica cochinchinensis
GGVCPKILKKCRRDSDCPGACICRGNGYCGSGSD

SEQ ID NO: 536 - protein, 34 aa, Momordica cochinchinensis
GGVCPKILAACRRDSDCPGACICRGNGYCGSGSD

SEQ ID NO: 537 - protein, 34 aa, Momordica cochinchinensis
GGVCPAILKKCRRDSDCPGACICRGNGYCGSGSD

SEQ ID NO: 538 - protein, 34 aa, Momordica cochinchinensis
GGVCPKILAKCRRDSDCPGACICRGNGYCGSGSD

SEQ ID NO: 539 - protein, 34 aa, Momordica cochinchinensis
GGVCPKILKACRRDSDCPGACICRGNGYCGSGSD

SEQ ID NO: 540 - protein, 29 aa, Otocinclus affinis
GLPVCGETCVGGTCNTPGCKCSWPVCTRN

SEQ ID NO: 541 - protein, 29 aa, Otocinclus affinis
GLPVCGETCVGGTCNTPGCTCSWPKCTRN

SEQ ID NO: 542 - protein, 14 aa, Helianthus annuus
GRCTKSIPPICFPD

SEQ ID NO: 543 - protein, 16 aa, Drosophila melanogaster
RQIKIWFQNRRMKWKK

SEQ ID NO: 544 - protein, 33 aa, Drosophila melanogaster
RQIKIWFQNRRMKWKKTYADFIASGRTGRRNAI

SEQ ID NO: 545 - protein, 13 aa, Human immunodeficiency virus 1
GRKKRRQRRRPPQ

SEQ ID NO: 546 - protein, 30 aa, Human immunodeficiency virus 1
GRKKRRQRRRPPQTYADFIASGRTGRRNAI

SEQ ID NO: 547 - protein, 16 aa, Drosophila melanogaster
RQIKIWFQNRRMKWKK

SEQ ID NO: 548 - protein, 16 aa, Drosophila melanogaster
RQIKIWFQNRRMKWKK

SEQ ID NO: 549 - protein, 16 aa, Drosophila melanogaster
RQIKIWFQNRRMKWKK

SEQ ID NO: 550 - protein, 16 aa, Drosophila melanogaster
RQIKIWFQNRRMKWKK

SEQ ID NO: 551 - protein, 22 aa, Homo sapiens
KCFQWQRNMRKVRGPPVSCIKR

SEQ ID NO: 552 - protein, 22 aa, Homo sapiens
KCFQWQRNMRKVRGPPVSCIKR

SEQ ID NO: 553 - protein, 22 aa, Homo sapiens
KCFQWQRNMRKVRGPPVSCIKR

SEQ ID NO: 554 - protein, 22 aa, Homo sapiens
KCFQWQRNMRKVRGPPVSCIKR

SEQ ID NO: 555 - protein, 14 aa, Human immunodeficiency virus 1
GRKKRRQRRRPPQC

SEQ ID NO: 556 - protein, 19 aa, Human immunodeficiency virus 1
TRQARRNRRRRWRERQRGC

SEQ ID NO: 557 - protein, 17 aa, Flock House virus
RRRRNRTRRNRRRVRGC

SEQ ID NO: 558 - protein, 21 aa, Brome mosaic virus
KMTRAQRRAAARRNRWTARGC

SEQ ID NO: 559 - protein, 15 aa, Human T-cell lymphotropic virus type 2
TRRQRTRRARRNRGC

SEQ ID NO: 560 - protein, 30 aa, Homo sapiens; identical to SEQ ID NO: 346

SEQ ID NO: 561 - protein, 28 aa, Homo sapiens
KRRIRRERNKMAAAKSRNRRRELTDTGC

SEQ ID NO: 562 - protein, 23 aa, Homo sapiens
WLRRIKAWLRRIKALNRQLGVAA

SEQ ID NO: 563 - protein, 10 aa, Human immunodeficiency virus 1
CRKKRRQRRR

SEQ ID NO: 564 - protein, 12 aa, Human immunodeficiency virus 1
GRKKRRQRRRPP

SEQ ID NO: 565 - protein, 4 aa, synthetic construct
  REGION 1..4: note = CPP
RRRR

SEQ ID NO: 566 - protein, 5 aa, synthetic construct
  REGION 1..5: note = CPP
RRRRR

SEQ ID NO: 567 - protein, 6 aa, synthetic construct
  REGION 1..6: note = CPP
RRRRRR

SEQ ID NO: 568 - protein, 7 aa, synthetic construct
  REGION 1..7: note = CPP
RRRRRRR

SEQ ID NO: 569 - protein, 8 aa, synthetic construct
  REGION 1..8: note = CPP
RRRRRRRR

SEQ ID NO: 570 - protein, 9 aa, synthetic construct
  REGION 1..9: note = CPP
RRRRRRRRR

SEQ ID NO: 571 - protein, 11 aa, synthetic construct
  REGION 1..11: note = CPP
RRRRRRRRRRR

SEQ ID NO: 572 - protein, 12 aa, synthetic construct
  REGION 1..12: note = CPP
RRRRRRRRRRRR

SEQ ID NO: 573 - protein, 16 aa, synthetic construct
  REGION 1..16: note = CPP
RRRRRRRRRRRRRRRR

SEQ ID NO: 574 - protein, 18 aa, synthetic construct
  REGION 1..18: note = CPP
KLALKLALKALKAALKLA

SEQ ID NO: 575 - protein, 18 aa, synthetic construct
  REGION 1..18: note = CPP
KLALKLALKAWKAALKLA

SEQ ID NO: 576 - protein, 18 aa, synthetic construct
  REGION 1..18: note = CPP
KLALKAALKAWKAAAKLA

SEQ ID NO: 577 - protein, 18 aa, synthetic construct
  REGION 1..18: note = CPP
KLALKAAAKAWKAAAKAA

SEQ ID NO: 578 - protein, 18 aa, synthetic construct
  REGION 1..18: note = CPP
KITLKLAIKAWKLALKAA

SEQ ID NO: 579 - protein, 18 aa, synthetic construct
  REGION 1..18: note = CPP
KIAAKSIAKIWKSILKIA

SEQ ID NO: 580 - protein, 18 aa, synthetic construct
  REGION 1..18: note = CPP
KALAKALAKLWKALAKAA

SEQ ID NO: 581 - protein, 18 aa, synthetic construct
  REGION 1..18: note = CPP
KLALKLALKWAKLALKAA

SEQ ID NO: 582 - protein, 18 aa, synthetic construct
  REGION 1..18: note = CPP
KLLAKAAKKWLLLALKAA

SEQ ID NO: 583 - protein, 18 aa, synthetic construct
  REGION 1..18: note = CPP
KLLAKAALKWLLKALKAA

SEQ ID NO: 584 - protein, 18 aa, synthetic construct
  REGION 1..18: note = CPP
KALKKLLAKWLAAAKALL

SEQ ID NO: 585 - protein, 18 aa, synthetic construct
  REGION 1..18: note = CPP
KLAAALLKKWKKLAAALL

SEQ ID NO: 586 - protein, 18 aa, synthetic construct
  REGION 1..18: note = CPP
KALAALLKKWAKLLAALK

SEQ ID NO: 587 - protein, 18 aa, synthetic construct
  REGION 1..18: note = CPP
KALAALLKKLAKLLAALK

SEQ ID NO: 588 - protein, 16 aa, synthetic construct
  REGION 1..16: note = CPP
KLALKLALKALKAALK

SEQ ID NO: 589 - protein, 14 aa, synthetic construct
  REGION 1..14: note = CPP
KLALKALKAALKLA

SEQ ID NO: 590 - protein, 14 aa, synthetic construct
  REGION 1..14: note = CPP
KLALKLALKALKAA

SEQ ID NO: 591 - protein, 18 aa, synthetic construct
  REGION 1..18: note = CPP
KLGLKLGLKGLKGGLKLG

SEQ ID NO: 592 - protein, 18 aa, synthetic construct
  REGION 1..18: note = CPP
KLALKLALKALQAALQLA

SEQ ID NO: 593 - protein, 18 aa, synthetic construct
  REGION 1..18: note = CPP
KLALQLALQALQAALQLA

SEQ ID NO: 594 - protein, 18 aa, synthetic construct
  REGION 1..18: note = CPP
QLALQLALQALQAALQLA

SEQ ID NO: 595 - protein, 18 aa, synthetic construct
  REGION 1..18: note = CPP
ELALELALEALEAALELA

SEQ ID NO: 596 - protein, 18 aa, synthetic construct
  REGION 1..18: note = CPP
LKTLATALTKLAKTLTTL

SEQ ID NO: 597 - protein, 18 aa, synthetic construct
  REGION 1..18: note = CPP
LLKTTALLKTTALLKTTA

SEQ ID NO: 598 - protein, 18 aa, synthetic construct
  REGION 1..18: note = CPP
LKTLTETLKELTKTLTEL

SEQ ID NO: 599 - protein, 18 aa, synthetic construct
  REGION 1..18: note = CPP
LLKTTELLKTTELLKTTE

SEQ ID NO: 600 - protein, 18 aa, synthetic construct
  REGION 1..18: note = CPP
KLALKLALKALKAALKLA

SEQ ID NO: 601 - protein, 18 aa, synthetic construct
  REGION 1..18: note = CPP
KALKLKLALALLAKLKLA

SEQ ID NO: 602 - protein, 8 aa, synthetic construct
  REGION 1..8: note = CPP
RRRRRRRW

SEQ ID NO: 603 - protein, 15 aa, synthetic construct
  REGION 1..15: note = CPP
KLWMRWYSPTTRRYG

SEQ ID NO: 604 - protein, 15 aa, synthetic construct
  REGION 1..15: note = CPP
DSLKSYWYLQKFSWR

SEQ ID NO: 605 - protein, 15 aa, synthetic construct
  REGION 1..15: note = CPP
RTLVNEYKNTLKFSK

SEQ ID NO: 606 - protein, 15 aa, synthetic construct
  REGION 1..15: note = CPP
IPSRWKDQFWKRWHY

SEQ ID NO: 607 - protein, 15 aa, synthetic construct
  REGION 1..15: note = CPP
GYGNCRHFKQKPRRD

SEQ ID NO: 608 - protein, 15 aa, synthetic construct
  REGION 1..15: note = CPP
KNAWKHSSCHHRHQI

SEQ ID NO: 609 - protein, 15 aa, synthetic construct
  REGION 1..15: note = CPP
RVREWWYTITLKQES

SEQ ID NO: 610 - protein, 15 aa, synthetic construct
  REGION 1..15: note = CPP
QQHLLIAINGYPRYN

SEQ ID NO: 611 - protein, 15 aa, synthetic construct
  REGION 1..15: note = CPP
WKCRRQCFRVLHHWN

SEQ ID NO: 612 - protein, 15 aa, synthetic construct
  REGION 1..15: note = CPP
RLWMRWYSPTTRRYG

SEQ ID NO: 613 - protein, 15 aa, synthetic construct
  REGION 1..15: note = CPP
KLWMRWYSATTRRYG

SEQ ID NO: 614 - protein, 15 aa, synthetic construct
  REGION 1..15: note = CPP
KLWMRWYSPWTRRYG

SEQ ID NO: 615 - protein, 15 aa, synthetic construct
  REGION 1..15: note = CPP
RLWMRWYSPWTRRYG

SEQ ID NO: 616 - protein, 15 aa, synthetic construct
  REGION 1..15: note = CPP
RLWMRWYSPWTRRWG

SEQ ID NO: 617 - protein, 15 aa, synthetic construct
  REGION 1..15: note = CPP
ALWMRWYSPTTRRYG

SEQ ID NO: 618 - protein, 15 aa, synthetic construct
  REGION 1..15: note = CPP
RAWMRWYSPTTRRYG

SEQ ID NO: 619 - protein, 15 aa, synthetic construct
  REGION 1..15: note = CPP
RLAMRWYSPTTRRYG

SEQ ID NO: 620 - protein, 15 aa, synthetic construct
  REGION 1..15: note = CPP
RLWARWYSPTTRRYG

SEQ ID NO: 621 - protein, 15 aa, synthetic construct
  REGION 1..15: note = CPP
RLWMAWYSPTTRRYG

SEQ ID NO: 622 - protein, 15 aa, synthetic construct
  REGION 1..15: note = CPP
RLWMRAYSPTTRRYG

SEQ ID NO: 623 - protein, 15 aa, synthetic construct
  REGION 1..15: note = CPP
RLWMRWASPTTRRYG

SEQ ID NO: 624 - protein, 15 aa, synthetic construct
  REGION 1..15: note = CPP
RLWMRWYAPTTRRYG

SEQ ID NO: 625 - protein, 15 aa, synthetic construct
  REGION 1..15: note = CPP
RLWMRWYSPATRRYG

SEQ ID NO: 626 - protein, 15 aa, synthetic construct
  REGION 1..15: note = CPP
RLWMRWYSPTARRYG

SEQ ID NO: 627 - protein, 15 aa, synthetic construct
  REGION 1..15: note = CPP
RLWMRWYSPTTARYG

SEQ ID NO: 628 - protein, 15 aa, synthetic construct
  REGION 1..15: note = CPP
RLWMRWYSPTTRAYG

SEQ ID NO: 629 - protein, 15 aa, synthetic construct
  REGION 1..15: note = CPP
RLWMRWYSPTTRRAG

SEQ ID NO: 630 - protein, 15 aa, synthetic construct
  REGION 1..15: note = CPP
RLWMRWYSPTTRRYA

SEQ ID NO: 631 - protein, 15 aa, synthetic construct
  REGION 1..15: note = CPP
RLLMRLYSPTTRRYG

SEQ ID NO: 632 - protein, 15 aa, synthetic construct
  REGION 1..15: note = CPP
RLFMRFYSPTTRRYG

SEQ ID NO: 633 - protein, 15 aa, synthetic construct
  REGION 1..15: note = CPP
RLIMRIYSPTTRRYG

SEQ ID NO: 634 - protein, 15 aa, synthetic construct
  REGION 1..15: note = CPP
RLVMRVYSPTTRRYG

SEQ ID NO: 635 - protein, 15 aa, synthetic construct
  REGION 1..15: note = CPP
RLYMRYYSPTTRRYG

SEQ ID NO: 636 - protein, 17 aa, synthetic construct
  REGION 1..17: note = CPP
DRDDRDDRDDRDDRDDR

SEQ ID NO: 637 - protein, 14 aa, synthetic construct
  REGION 1..14: note = CPP
ERERERERERERER

SEQ ID NO: 638 - protein, 14 aa, synthetic construct
  REGION 1..14: note = CPP
WRWRWRWRWRWRWR

SEQ ID NO: 639 - protein, 10 aa, synthetic construct
  REGION 1..10: note = CPP
DRDRDRDRDR

SEQ ID NO: 640 - protein, 27 aa, synthetic construct
  REGION 1..27: note = CPP
GALFLGFLGAAGSTMGAWSQPKKKRKV

SEQ ID NO: 641 - protein, 4 aa, synthetic construct
  REGION 1..4: note = CPP
EEEA

SEQ ID NO: 642 - protein, 5 aa, synthetic construct
  REGION 1..5: note = CPP
EEEAA

SEQ ID NO: 643 - protein, 8 aa, synthetic construct
  REGION 1..8: note = CPP
EEEAAKKK

SEQ ID NO: 644 - protein, 16 aa, synthetic construct
  REGION 1..16: note = CPP
KTVLLRKLLKLLVRKI

SEQ ID NO: 645 - protein, 16 aa, synthetic construct
  REGION 1..16: note = CPP
LLKKRKVVRLIKFLLK

SEQ ID NO: 646 - protein, 9 aa, synthetic construct
  REGION 1..9: note = CPP
CGNKRTRGC

SEQ ID NO: 647 - protein, 12 aa, synthetic construct
  REGION 1..12: note = CPP
TSPLNIHNGQKL

SEQ ID NO: 648 - protein, 13 aa, synthetic construct
  REGION 1..13: note = CPP
CVQWSLLRGYQPC

SEQ ID NO: 649 - protein, 26 aa, synthetic construct
  REGION 1..26: note = CPP
DPKGDPKGVTVTVTVTVTGKGDPKPD

SEQ ID NO: 650 - protein, 16 aa, synthetic construct
  REGION 1..16: note = CPP
YTAIAWVKAFIRKLRK

SEQ ID NO: 651 - protein, 17 aa, synthetic construct
  REGION 1..17: note = CPP
IAWVKAFIRKLRKGPLG

SEQ ID NO: 652 - protein, 34 aa, synthetic construct
  REGION 1..34: note = CPP
KKKKKKGGFLGFWRGENGRKTRSAYERMCILKGK

SEQ ID NO: 653 - protein, 14 aa, synthetic construct
  REGION 1..14: note = CPP
RLSGMNEVLSFRWL

SEQ ID NO: 654 - protein, 13 aa, synthetic construct
  REGION 1..13: note = CPP
GPFHFYQFLFPPV

SEQ ID NO: 655 - protein, 13 aa, synthetic construct
  REGION 1..13: note = CPP
GSPWGLQHHPPRT

SEQ ID NO: 656 - protein, 30 aa, synthetic construct; identical to SEQ ID NO: 28
  REGION 1..30: note = CPP

SEQ ID NO: 657 - protein, 30 aa, synthetic construct
  REGION 1..30: note = CPP
WEAALAEALAEALAEHLAEALAEALEALAA

SEQ ID NO: 658 - protein, 20 aa, synthetic construct
  REGION 1..20: note = CPP
GLFEALLELLESLWELLLEA

SEQ ID NO: 659 - protein, 20 aa, synthetic construct
  REGION 1..20: note = CPP
GLFKALLKLLKSLWKLLLKA

SEQ ID NO: 660 - protein, 20 aa, synthetic construct
  REGION 1..20: note = CPP
GLFRALLRLLRSLWRLLLRA

SEQ ID NO: 661 - protein, 11 aa, synthetic construct
  REGION 1..11: note = CPP
YARAAARQARA

SEQ ID NO: 662 - protein, 10 aa, synthetic construct
  REGION 1..10: note = CPP
GKKKKKKKKK

SEQ ID NO: 663 - protein, 20 aa, synthetic construct
  REGION 1..20: note = CPP
RILQQLLFIHFRIGCRHSRI

SEQ ID NO: 664 - protein, 17 aa, synthetic construct
  REGION 1..17: note = CPP
RILQQLLFIHFRIGCRH

SEQ ID NO: 665 - protein, 15 aa, synthetic construct
  REGION 1..15: note = CPP
RILQQLLFIHFRIGC

SEQ ID NO: 666 - protein, 10 aa, synthetic construct
  REGION 1..10: note = CPP
RIFIHFRIGC

SEQ ID NO: 667 - protein, 8 aa, synthetic construct
  REGION 1..8: note = CPP
RIFIRIGC

SEQ ID NO: 668 - protein, 11 aa, synthetic construct
  REGION 1..11: note = CPP
RILQQLLFIHF

SEQ ID NO: 669 - protein, 6 aa, synthetic construct
  REGION 1..6: note = CPP
RIFIGC

SEQ ID NO: 670 - protein, 6 aa, synthetic construct
  REGION 1..6: note = CPP
FIRIGC

SEQ ID NO: 671 - protein, 22 aa, synthetic construct
  REGION 1..22: note = CPP
DTWAGVEAIIRILQQLLFIHFR

SEQ ID NO: 672 - protein, 5 aa, synthetic construct
  REGION 1..5: note = CPP
IGCRH

SEQ ID NO: 673 - protein, 23 aa, synthetic construct
  REGION 1..23: note = CPP
GYGRKKRRGRRRTHRLPRRRRRR

SEQ ID NO: 674 - protein, 12 aa, synthetic construct
  REGION 1..12: note = CPP
KRIIQRILSRNS

SEQ ID NO: 675 - protein, 12 aa, synthetic construct
  REGION 1..12: note = CPP
KRIHPRLTRSIR

SEQ ID NO: 676 - protein, 12 aa, synthetic construct
  REGION 1..12: note = CPP
PPRLRKRRQLNM

SEQ ID NO: 677 - protein, 12 aa, synthetic construct
  REGION 1..12: note = CPP
PIRRRKKLRRLK

SEQ ID NO: 678 - protein, 12 aa, synthetic construct
  REGION 1..12: note = CPP
RRQRRTSKLMKR

SEQ ID NO: 679 - protein, 12 aa, synthetic construct
  REGION 1..12: note = CPP
MHKRPTTPSRKM

SEQ ID NO: 680 - protein, 12 aa, synthetic construct
  REGION 1..12: note = CPP
RQRSRRRPLNIR

SEQ ID NO: 681 - protein, 12 aa, synthetic construct
  REGION 1..12: note = CPP
RIRMIQNLIKKT

SEQ ID NO: 682 - protein, 12 aa, synthetic construct
  REGION 1..12: note = CPP
SRRKRQRSNMRI

SEQ ID NO: 683 - protein, 12 aa, synthetic construct
  REGION 1..12: note = CPP
QRIRKSKISRTL

SEQ ID NO: 684 - protein, 12 aa, synthetic construct
  REGION 1..12: note = CPP
PSKRLLHNNLRR

SEQ ID NO: 685 - protein, 12 aa, synthetic construct
  REGION 1..12: note = CPP
HRHIRRQSLIML

SEQ ID NO: 686 - protein, 12 aa, synthetic construct
  REGION 1..12: note = CPP
PQNRLQIRRHSK

SEQ ID NO: 687 - protein, 12 aa, synthetic construct
  REGION 1..12: note = CPP
PPHNRIQRRLNM

SEQ ID NO: 688 - protein, 12 aa, synthetic construct
  REGION 1..12: note = CPP
SMLKRNHSTSNR

SEQ ID NO: 689 - protein, 12 aa, synthetic construct
  REGION 1..12: note = CPP
GSRHPSLIIPRQ

SEQ ID NO: 690 - protein, 12 aa, synthetic construct
  REGION 1..12: note = CPP
SPMQKTMNLPPM

SEQ ID NO: 691 - protein, 12 aa, synthetic construct
  REGION 1..12: note = CPP
NKRILIRIMTRP

SEQ ID NO: 692 - protein, 12 aa, synthetic construct
  REGION 1..12: note = CPP
HGWZIHGLLHRA

SEQ ID NO: 693 - protein, 11 aa, synthetic construct
  REGION 1..11: note = CPP
AVPAKKRZKSV

SEQ ID NO: 694 - protein, 11 aa, synthetic construct
  REGION 1..11: note = CPP
PNTRVRPDVSF

SEQ ID NO: 695 - protein, 12 aa, synthetic construct
  REGION 1..12: note = CPP
LTRNYEAWVPTP

SEQ ID NO: 696 - protein, 13 aa, synthetic construct
  REGION 1..13: note = CPP
SAETVESCLAKSH

SEQ ID NO: 697 - protein, 12 aa, synthetic construct
  REGION 1..12: note = CPP
YSHIATLPFTPT

SEQ ID NO: 698 - protein, 12 aa, synthetic construct
  REGION 1..12: note = CPP
SYIQRTPSTTLP

SEQ ID NO: 699 - protein, 12 aa, synthetic construct
  REGION 1..12: note = CPP
AVPAENALNNPF

SEQ ID NO: 700 - protein, 12 aa, synthetic construct
  REGION 1..12: note = CPP
SFHQFARATLAS

SEQ ID NO: 701 - protein, 12 aa, synthetic construct
  REGION 1..12: note = CPP
QSPTDFTFPNPL

SEQ ID NO: 702 - protein, 12 aa, synthetic construct
  REGION 1..12: note = CPP
HFAAWGGWSLVH

SEQ ID NO: 703 - protein, 12 aa, synthetic construct
  REGION 1..12: note = CPP
HIQLSPFSQSWR

SEQ ID NO: 704 - protein, 12 aa, synthetic construct
  REGION 1..12: note = CPP
LTMPSDLQPVLW

SEQ ID NO: 705 - protein, 12 aa, synthetic construct
  REGION 1..12: note = CPP
FQPYDHPAEVSY

SEQ ID NO: 706 - protein, 12 aa, synthetic construct
  REGION 1..12: note = CPP
FDPFFWKYSPRD

SEQ ID NO: 707 - protein, 12 aa, synthetic construct
  REGION 1..12: note = CPP
FAPWDTASFMLG

SEQ ID NO: 708 - protein, 12 aa, synthetic construct
  REGION 1..12: note = CPP
FTYKNFFWLPEL

SEQ ID NO: 709 - protein, 12 aa, synthetic construct
  REGION 1..12: note = CPP
SATGAPWKMWVR

SEQ ID NO: 710 - protein, 12 aa, synthetic construct
  REGION 1..12: note = CPP
SLGWMLPFSPPF

SEQ ID NO: 711 - protein, 12 aa, synthetic construct
  REGION 1..12: note = CPP
SHAFTWPTYLQL

SEQ ID NO: 712 - protein, 12 aa, synthetic construct
  REGION 1..12: note = CPP
SHNWLPLWPLRP

SEQ ID NO: 713 - protein, 12 aa, synthetic construct
  REGION 1..12: note = CPP
SWLPYPWHVPSS

SEQ ID NO: 714 - protein, 12 aa, synthetic construct
  REGION 1..12: note = CPP
SWWTPWHVHSES

SEQ ID NO: 715 - protein, 12 aa, synthetic construct
  REGION 1..12: note = CPP
SWAQHLSLPPVL

SEQ ID NO: 716 - protein, 12 aa, synthetic construct
  REGION 1..12: note = CPP
SSSIFPPWLSFF

SEQ ID NO: 717 - protein, 12 aa, synthetic construct
  REGION 1..12: note = CPP
LNVPPSWFLSQR

SEQ ID NO: 718 - protein, 12 aa, synthetic construct
  REGION 1..12: note = CPP
LDITPFLSLTLP

SEQ ID NO: 719 - protein, 12 aa, synthetic construct
  REGION 1..12: note = CPP
LPHPVLHMGPLR

SEQ ID NO: 720 - protein, 12 aa, synthetic construct
  REGION 1..12: note = CPP
VSKQPYYMWNGN

SEQ ID NO: 721 - protein, 12 aa, synthetic construct
  REGION 1..12: note = CPP
NYTTYKSHFQDR

SEQ ID NO: 722 - protein, 12 aa, synthetic construct
  REGION 1..12: note = CPP
AIPNNQLGFPFK

SEQ ID NO: 723 - protein, 12 aa, synthetic construct
  REGION 1..12: note = CPP
NIENSTLATPLS

SEQ ID NO: 724 - protein, 12 aa, synthetic construct
  REGION 1..12: note = CPP
YPYDANHTRSPT

SEQ ID NO: 725 - protein, 12 aa, synthetic construct
  REGION 1..12: note = CPP
DPATNPGPHFPR

SEQ ID NO: 726 - protein, 12 aa, synthetic construct
  REGION 1..12: note = CPP
TLPSPLALLTVH

SEQ ID NO: 727 - protein, 12 aa, synthetic construct
  REGION 1..12: note = CPP
HPGSPFPPEHRP

SEQ ID NO: 728 - protein, 12 aa, synthetic construct
  REGION 1..12: note = CPP
TSHTDAPPARSP

SEQ ID NO: 729 - protein, 12 aa, synthetic construct
  REGION 1..12: note = CPP
MTPSSLSTLPWP

SEQ ID NO: 730 - protein, 12 aa, synthetic construct
  REGION 1..12: note = CPP
VLGQSGYLMPMR

SEQ ID NO: 731 - protein, 12 aa, synthetic construct
  REGION 1..12: note = CPP
QPIIITSPYLPS

SEQ ID NO: 732 - protein, 12 aa, synthetic construct
  REGION 1..12: note = CPP
TPKTMTQTYDFS

SEQ ID NO: 733 - protein, 12 aa, synthetic construct
  REGION 1..12: note = CPP
NSGTMQSASRAT

SEQ ID NO: 734 - protein, 12 aa, synthetic construct
  REGION 1..12: note = CPP
QAASRVENYMHR

SEQ ID NO: 735 - protein, 12 aa, synthetic construct
  REGION 1..12: note = CPP
HQHKPPPLTNNW

SEQ ID NO: 736 - protein, 12 aa, synthetic construct
  REGION 1..12: note = CPP
SNPWDSLLSVST

SEQ ID NO: 737 - protein, 12 aa, synthetic construct
  REGION 1..12: note = CPP
KTIEAHPPYYAS

SEQ ID NO: 738 - protein, 12 aa, synthetic construct
  REGION 1..12: note = CPP
EPDNWSLDFPRR

SEQ ID NO: 739 - protein, 12 aa, synthetic construct
  REGION 1..12: note = CPP
HQHKPPPLTNNW

SEQ ID NO: 740 - protein, 20 aa, synthetic construct
  REGION 1..20: note = CPP
GLWRALWRLLRSLWRLLWKA

SEQ ID NO: 741 - protein, 21 aa, synthetic construct
  REGION 1..21: note = CPP
GLWRALWRALWRSLWKLKRKV

SEQ ID NO: 742 - protein, 20 aa, synthetic construct
  REGION 1..20: note = CPP
GLWRALWRALRSLWKLKRKV

SEQ ID NO: 743 - protein, 20 aa, synthetic construct
  REGION 1..20: note = CPP
GLWRALWRGLRSLWKLKRKV

SEQ ID NO: 744 - protein, 20 aa, synthetic construct
  REGION 1..20: note = CPP
GLWRALWRGLRSLWKKKRKV

SEQ ID NO: 745 - protein, 21 aa, synthetic construct
  REGION 1..21: note = CPP
GLWRALWRALWRSLWKLKWKV

SEQ ID NO: 746 - protein, 21 aa, synthetic construct
  REGION 1..21: note = CPP
GLWRALWRALWRSLWKSKRKV

SEQ ID NO: 747 - protein, 21 aa, synthetic construct
  REGION 1..21: note = CPP
GLWRALWRALWRSLWKKKRKV

SEQ ID NO: 748 - protein, 21 aa, synthetic construct
  REGION 1..21: note = CPP
GLWRALWRALWRSLWKLKRKV

SEQ ID NO: 749 - protein, 27 aa, synthetic construct
  REGION 1..27: note = CPP
GLWRALWRLLRSLWRLLWSQPKKKRKV

SEQ ID NO: 750 - protein, 11 aa, synthetic construct
  REGION 1..11: note = CPP
YARAARRAARR

SEQ ID NO: 751 - protein, 11 aa, synthetic construct
  REGION 1..11: note = CPP
PARAARRAARR

SEQ ID NO: 752 - protein, 11 aa, synthetic construct
  REGION 1..11: note = CPP
YPRAARRAARR

SEQ ID NO: 753 - protein, 11 aa, synthetic construct
  REGION 1..11: note = CPP
YRRAARRAARA

SEQ ID NO: 754 - protein, 11 aa, synthetic construct
  REGION 1..11: note = CPP
YGRAARRAARR

SEQ ID NO: 755 - protein, 11 aa, synthetic construct
  REGION 1..11: note = CPP
YAREARRAARR

SEQ ID NO: 756 - protein, 11 aa, synthetic construct
  REGION 1..11: note = CPP
YEREARRAARR

SEQ ID NO: 757 - protein, 11 aa, synthetic construct
  REGION 1..11: note = CPP
YKRAARRAARR

SEQ ID NO: 758 - protein, 11 aa, synthetic construct
  REGION 1..11: note = CPP
YARKARRAARR

SEQ ID NO: 759 - protein, 11 aa, synthetic construct
  REGION 1..11: note = CPP
YKRKARRAARR

SEQ ID NO: 760 - protein, 11 aa, synthetic construct
  REGION 1..11: note = CPP
YGRRARRAARR

SEQ ID NO: 761 - protein, 11 aa, synthetic construct
  REGION 1..11: note = CPP
YGRRARRRARR

SEQ ID NO: 762 - protein, 11 aa, synthetic construct
  REGION 1..11: note = CPP
YGRRARRRRRR

SEQ ID NO: 763 - protein, 11 aa, synthetic construct
  REGION 1..11: note = CPP
YGRRRRRRRRR

SEQ ID NO: 764 - protein, 11 aa, synthetic construct
  REGION 1..11: note = CPP
YRRRRRRRRRR

SEQ ID NO: 765 - protein, 16 aa, synthetic construct
  REGION 1..16: note = CPP
GKINLKALAALAKKIL

SEQ ID NO: 766 - protein, 16 aa, synthetic construct
  REGION 1..16: note = CPP
RVIRVWFQNKRCKDKK

SEQ ID NO: 767 - protein, 39 aa, synthetic construct
  REGION 1..39: note = CPP
GRKKRRQRRRPPQGRKKRRQRRRPPQGRKKRRQRRRPPQ

SEQ ID NO: 768 - protein, 22 aa, synthetic construct
  REGION 1..22: note = CPP
GEQIAQLIAGYIDIILKKKKSK

SEQ ID NO: 769 - protein, 14 aa, synthetic construct
  REGION 1..14: note = CPP
GRKKRRQRRRPPQC

SEQ ID NO: 770 - protein, 28 aa, synthetic construct
  REGION 1..28: note = CPP
AAVALLPAVLLALLAPRKKRRQRRRPPQ

SEQ ID NO: 771 - protein, 29 aa, synthetic construct
  REGION 1..29: note = CPP
AAVALLPAVLLALLAPRKKRRQRRRPPQC

SEQ ID NO: 772 - protein, 28 aa, synthetic construct
  REGION 1..28: note = CPP
AAVALLPAVLLALLAPRKKRRQRRRPPQ

SEQ ID NO: 773 - protein, 29 aa, synthetic construct
  REGION 1..29: note = CPP
RKKRRQRRRPPQCAAVALLPAVLLALLAP

SEQ ID NO: 774 - protein, 29 aa, synthetic construct
  REGION 1..29: note = CPP
RRRQRRKRGGDIMGEWGNEIFGAIAGFLG

SEQ ID NO: 775 - protein, 29 aa, synthetic construct
  REGION 1..29: note = CPP
RRRQRRKRGGDIMGEWGNEIFGAIAGFLG

SEQ ID NO: 776 - protein, 25 aa, synthetic construct
  REGION 1..25: note = CPP
YGRKKRRQRRRGCYGRKKRRQRRRG

SEQ ID NO: 777 - protein, 13 aa, synthetic construct
  REGION 1..13: note = CPP
GRKKRRQRRRPPQ

SEQ ID NO: 778 - protein, 22 aa, synthetic construct
  REGION 1..22: note = CPP
AAVALLPAVLLALLAPRRRRRR

SEQ ID NO: 779 - protein, 16 aa, synthetic construct
  REGION 1..16: note = CPP
RLWRALPRVLRRLLRP

SEQ ID NO: 780 - protein, 28 aa, synthetic construct
  REGION 1..28: note = CPP
AAVALLPAVLLALLAPSGASGLDKRDYV

SEQ ID NO: 781 - protein, 33 aa, synthetic construct
  REGION 1..33: note = CPP
LLETLLKPFQCRICMRNFSTRQARRNHRRRHRR

SEQ ID NO: 782 - protein, 27 aa, synthetic construct
  REGION 1..27: note = CPP
AAVACRICMRNFSTRQARRNHRRRHRR

SEQ ID NO: 783 - protein, 16 aa, synthetic construct
  REGION 1..16: note = CPP
RQIKIWFQNRRMKWKK

SEQ ID NO: 784 - protein, 16 aa, synthetic construct
  REGION 1..16: note = CPP
RQIKIWFQNRRMKWKK

SEQ ID NO: 785 - protein, 35 aa, synthetic construct
  REGION 1..35: note = CPP
RQIKIWFQNRRMKWKKDIMGEWGNEIFGAIAGFLG

SEQ ID NO: 786 - protein, 37 aa, synthetic construct
  REGION 1..37: note = CPP
SGRGKQGGKARAKAKTRSSRAGLQFPVGRVHRLLRKG

SEQ ID NO: 787 - protein, 38 aa, synthetic construct
  REGION 1..38: note = CPP
SGRGKQGGKARAKAKTRSSRAGLQFPVGRVHRLLRKGC

SEQ ID NO: 788 - protein, 25 aa, synthetic construct
  REGION 1..25: note = CPP
KKDGKKRKRSRKESYSVYVYKVLKQ

SEQ ID NO: 789 - protein, 36 aa, synthetic construct
  REGION 1..36: note = CPP
KGSKKAVTKAQKKDGKKRKRSRKESYSVYVYKVLKQ

SEQ ID NO: 790 - protein, 27 aa, synthetic construct
  REGION 1..27: note = CPP
GWTLNSAGYLLGKINLKALAALAKKIL

SEQ ID NO: 791 - protein, 18 aa, synthetic construct
  REGION 1..18: note = CPP
KLALKLALKALKAALKLA

SEQ ID NO: 792 - protein, 21 aa, synthetic construct
  REGION 1..21: note = CPP
KETWWETWWTEWSQPKKKRKV

SEQ ID NO: 793 - protein, 28 aa, synthetic construct
  REGION 1..28: note = CPP
KETWWETWWTEWSQPGRKKRRQRRRPPQ

SEQ ID NO: 794 - protein, 15 aa, synthetic construct
  REGION 1..15: note = CPP
RVIRWFQNKRCKDKK

SEQ ID NO: 795 - protein, 18 aa, synthetic construct
  REGION 1..18: note = CPP
LGLLLRHLRHHSNLLANI

SEQ ID NO: 796 - protein, 16 aa, synthetic construct
  REGION 1..16: note = CPP
KLWSAWPSLWSSLWKP

SEQ ID NO: 797 - protein, 22 aa, synthetic construct
  REGION 1..22: note = CPP
GLGSLLKKAGKKLKQPKSKRKV

SEQ ID NO: 798 - protein, 12 aa, synthetic construct
  REGION 1..12: note = CPP
FKQQQQQQQQQQ

SEQ ID NO: 799 - protein, 4 aa, synthetic construct
  REGION 1..4: note = CPP
YRFK

SEQ ID NO: 800 - protein, 13 aa, synthetic construct
  REGION 1..13: note = CPP
YRFKYRFKYRLFK

SEQ ID NO: 801 - protein, 10 aa, synthetic construct
  REGION 1..10: note = CPP
WRFKKSKRKV

SEQ ID NO: 802 - protein, 20 aa, synthetic construct
  REGION 1..20: note = CPP
WRFKAAVALLPAVLLALLAP

SEQ ID NO: 803 - protein, 8 aa, synthetic construct
  REGION 1..8: note = CPP
WRFKWRFK

SEQ ID NO: 804 - protein, 12 aa, synthetic construct
  REGION 1..12: note = CPP
WRFKWRFKWRFK

SEQ ID NO: 805 - protein, 36 aa, synthetic construct; identical to SEQ ID NO: 789
  REGION 1..36: note = CPP

SEQ ID NO: 806 - protein, 36 aa, synthetic construct
  REGION 1..36: note = CPP
RGSRRAVTRAQRRDGRRRRRSRRESYSVYVYRVLRQ

SEQ ID NO: 807 - protein, 15 aa, synthetic construct
  REGION 1..15: note = CPP
RVIRWFQNKRSKDKK

SEQ ID NO: 808 - protein, 27 aa, synthetic construct
  REGION 1..27: note = CPP
GWTLNSAGYLLGKINLKALAALAKKIL

SEQ ID NO: 809 - protein, 28 aa, synthetic construct
  REGION 1..28: note = CPP
AAVALLPAVLLALLAPRKKRRQRRRPPQ

SEQ ID NO: 810 - protein, 5 aa, synthetic construct
  REGION 1..5: note = CPP
CWKKK

SEQ ID NO: 811 - protein, 10 aa, synthetic construct
  REGION 1..10: note = CPP
CWKKKKKKKK

SEQ ID NO: 812 - protein, 15 aa, synthetic construct
  REGION 1..15: note = CPP
CWKKKKKKKKKKKKK

SEQ ID NO: 813 - protein, 20 aa, synthetic construct
  REGION 1..20: note = CPP
CWKKKKKKKKKKKKKKKKKK

SEQ ID NO: 814 - protein, 19 aa, synthetic construct
  REGION 1..19: note = CPP
KKKKKKKKKKKKKKKKKKK

SEQ ID NO: 815 - protein, 19 aa, synthetic construct
  REGION 1..19: note = CPP
KKWKMRRGAGRRRRRRRRR

SEQ ID NO: 816 - protein, 12 aa, synthetic construct
  REGION 1..12: note = CPP
APWHLSSQYSRT

SEQ ID NO: 817 - protein, 11 aa, synthetic construct
  REGION 1..11: note = CPP
RRRRRRRRRRR

SEQ ID NO: 818 - protein, 20 aa, synthetic construct
  REGION 1..20: note = CPP
KLALKLALKALKAALKLAGC

SEQ ID NO: 819 - protein, 15 aa, synthetic construct
  REGION 1..15: note = CPP
KLLKLLKLKALKLGC

SEQ ID NO: 820 - protein, 35 aa, synthetic construct
  REGION 1..35: note = CPP
GGGARKKAAKAARKKAAKAARKKAAKAARKKAAKA

SEQ ID NO: 821 - protein, 10 aa, synthetic construct
  REGION 1..10: note = CPP
CRRRRRRRRR

SEQ ID NO: 822 - protein, 10 aa, synthetic construct
  REGION 1..10: note = CPP
CKKKKKKKKK

SEQ ID NO: 823 - protein, 27 aa, synthetic construct
  REGION 1..27: note = CPP
GWTLNSAGYLLGKINLKALAALAKKIL

SEQ ID NO: 824 - protein, 27 aa, synthetic construct
  REGION 1..27: note = CPP
GWTLNSAGYLLGKINLKALAALAKKLL

SEQ ID NO: 825 - protein, 26 aa, synthetic construct
  REGION 1..26: note = CPP
GWTLNSAGYLLGKFLPLILRKIVTAL

SEQ ID NO: 826 - protein, 27 aa, synthetic construct
  REGION 1..27: note = CPP
GWTLNPAGYLLGKINLKALAALAKKIL

SEQ ID NO: 827 - protein, 27 aa, synthetic construct
  REGION 1..27: note = CPP
GWTLNPPGYLLGKINLKALAALAKKIL

SEQ ID NO: 828 - protein, 24 aa, synthetic construct
  REGION 1..24: note = CPP
LNSAGYLLGKINLKALAALAKKIL

SEQ ID NO: 829 - protein, 18 aa, synthetic construct
  REGION 1..18: note = CPP
LLGKINLKALAALAKKIL

SEQ ID NO: 830 - protein, 25 aa, synthetic construct
  REGION 1..25: note = CPP
GWTLNSAGYLLGKLKALAALAKKIL

SEQ ID NO: 831 - protein, 21 aa, synthetic construct
  REGION 1..21: note = CPP
AGYLLGKINLKALAALAKKIL

SEQ ID NO: 832 - protein, 21 aa, synthetic construct
  REGION 1..21: note = CPP
GWTLNSKINLKALAALAKKIL

SEQ ID NO: 833 - protein, 21 aa, synthetic construct
  REGION 1..21: note = CPP
LNSAGYLLGKLKALAALAKIL

SEQ ID NO: 834 - protein, 20 aa, synthetic construct
  REGION 1..20: note = CPP
LNSAGYLLGKALAALAKKIL

SEQ ID NO: 835 - protein, 19 aa, synthetic construct
  REGION 1..19: note = CPP
AGYLLGKLKALAALAKKIL

SEQ ID NO: 836 - protein, 19 aa, synthetic construct
  REGION 1..19: note = CPP
LNSAGYLLGKLKALAALAK

SEQ ID NO: 837 - protein, 27 aa, synthetic construct
  REGION 1..27: note = CPP
GWTLNSAGYLLGKINLKAPAALAKKIL

SEQ ID NO: 838 - protein, 21 aa, synthetic construct
  REGION 1..21: note = CPP
AGYLLGKINLKALAALAKKIL

SEQ ID NO: 839 - protein, 21 aa, synthetic construct
  REGION 1..21: note = CPP
KETWWETWWTEWSQPKKKRKV

SEQ ID NO: 840 - protein, 21 aa, synthetic construct
  REGION 1..21: note = CPP
KETWFETWFTEWSQPKKKRKV

SEQ ID NO: 841 - protein, 15 aa, synthetic construct
  REGION 1..15: note = CPP
KWFETWFTEWPKKRK

SEQ ID NO: 842 - protein, 27 aa, synthetic construct
  REGION 1..27: note = CPP
GALFLGFLGAAGSTMGAWSQPKKKRKV

SEQ ID NO: 843 - protein, 27 aa, synthetic construct
  REGION 1..27: note = CPP
GALFLGFLGAAGSTMGAWSQPKSKRKV

SEQ ID NO: 844 - protein, 20 aa, synthetic construct
  REGION 1..20: note = CPP
ALWKTLLKKVLKAPKKKRKV

SEQ ID NO: 845 - protein, 20 aa, synthetic construct
  REGION 1..20: note = CPP
PKKKRKVALWKTLLKKVLKA

SEQ ID NO: 846 - protein, 20 aa, synthetic construct
  REGION 1..20: note = CPP
VKRKKKPALWKTLLKKVLKA

SEQ ID NO: 847 - protein, 22 aa, synthetic construct
  REGION 1..22: note = CPP
RQARRNRRRALWKTLLKKVLKA

SEQ ID NO: 848 - protein, 13 aa, synthetic construct
  REGION 1..13: note = CPP
EEEAAGRKRKKRT

SEQ ID NO: 849 - protein, 13 aa, synthetic construct
  REGION 1..13: note = CPP
FFFAAGRKRKKRT

SEQ ID NO: 850 - protein, 13 aa, synthetic construct
  REGION 1..13: note = CPP
NNNAAGRKRKKRT

SEQ ID NO: 851 - protein, 13 aa, synthetic construct
  REGION 1..13: note = CPP
YYYAAGRKRKKRT

SEQ ID NO: 852 - protein, 21 aa, synthetic construct
  REGION 1..21: note = CPP
AGYLLGKINLKALAALAKKIL

SEQ ID NO: 853 - protein, 27 aa, synthetic construct
  REGION 1..27: note = CPP
GALFLGFLGAAGSTMGAWSQPKKKRKV

SEQ ID NO: 854 - protein, 27 aa, synthetic construct
  REGION 1..27: note = CPP
GALFLAFLAAALSLMGLWSQPKKKRRV

SEQ ID NO: 855 - protein, 27 aa, synthetic construct
  REGION 1..27: note = CPP
GLLEALAELLEGLRKRLRKFRNKIKEK

SEQ ID NO: 856 - protein, 41 aa, synthetic construct
  REGION 1..41: note = CPP
AAVALLPAVLLALLAPEILLPNNYNAYESYKYPGMFIALSK

SEQ ID NO: 857 - protein, 26 aa, synthetic construct
  REGION 1..26: note = CPP
AAVALLPAVLLALLAPVQRKRQKLMP

SEQ ID NO: 858 - protein, 27 aa, synthetic construct
  REGION 1..27: note = CPP
MGLGLHLLVLAAALQGAWSQPKKKRKV

SEQ ID NO: 859 - protein, 23 aa, synthetic construct
  REGION 1..23: note = CPP
MGLGLHLLVLAAALQGAKKKRKV

SEQ ID NO: 860 - protein, 26 aa, synthetic construct
  REGION 1..26: note = CPP
AAVALLPAVLLALLAKKNNLKDCGLF

SEQ ID NO: 861 - protein, 26 aa, synthetic construct
  REGION 1..26: note = CPP
AAVALLPAVLLALLAKKNNLKECGLY

SEQ ID NO: 862 - protein, 43 aa, synthetic construct
  REGION 1..43: note = CPP
AAVALLPAVLLALLAVTDQLGEDFFAVDLEAFLQEFGLLPEKE

SEQ ID NO: 863 - protein, 21 aa, synthetic construct
  REGION 1..21: note = CPP
AGYLLGKINLKALAALAKKIL

SEQ ID NO: 864 - protein, 38 aa, synthetic construct
  REGION 1..38: note = CPP
AGYLLGKINLKALAALAKKILTYADFIASGRTGRRNAI

SEQ ID NO: 865 - protein, 28 aa, synthetic construct
  REGION 1..28: note = CPP
RRRRRRRRRRRTYADFIASGRTGRRNAI

SEQ ID NO: 866 - protein, 22 aa, synthetic construct
  REGION 1..22: note = R41
  SITE 22: note = MISC_FEATURE - Amide
FILFILFILGGKHKHKHKHKHK

SEQ ID NO: 867 - protein, 22 aa, synthetic construct
  REGION 1..22: note = R8
  SITE 22: note = MISC_FEATURE - Amide
GPPRFPPRFPPRFPPRFPPRFP

SEQ ID NO: 868 - protein, 22 aa, synthetic construct
  REGION 1..22: note = M918
  SITE 22: note = MISC_FEATURE - amide
MVTVLFRRLRIRRASGPPRVRV

SEQ ID NO: 869 - protein, 17 aa, synthetic construct
  REGION 1..17: note = YTA-4
  SITE 1: note = MISC_FEATURE - Acetyl
  SITE 17: note = MISC_FEATURE - amide
IAWVKAFIRKLRKGPLG

SEQ ID NO: 870 - protein, 8 aa, synthetic construct
  REGION 1..8: note = cyclic CPP
  REGION 1..8: note = MISC_FEATURE - cyclic
  REGION 1..8: note = MISC_FEATURE - may be cyclic
RRRRWWWW

SEQ ID NO: 871 - protein, 7 aa, synthetic construct
  REGION 1..7: note = Cyclic CPP
  REGION 1..7: note = MISC_FEATURE - may be cyclic
RRRRWWW

SEQ ID NO: 872 - protein, 8 aa, synthetic construct
  REGION 1..8: note = cyclic CPP
  REGION 1..8: note = MISC_FEATURE - cyclic
EEEEWWWW

SEQ ID NO: 873 - protein, 7 aa, synthetic construct
  REGION 1..7: note = cyclic CPP
  REGION 1..7: note = MISC_FEATURE - cyclic
EEEEWWW

SEQ ID NO: 874 - protein, 6 aa, synthetic construct
  REGION 1..6: note = cyclic CPP
  SITE 1: note = MISC_FEATURE - may comprise a fatty acyl group containing at least 8 carbons
  REGION 1..6: note = MISC_FEATURE - may be cyclic
KRRRRR

SEQ ID NO: 875 - protein, 7 aa, synthetic construct
  REGION 1..7: note = cyclic CPP
  SITE 1: note = MISC_FEATURE - may comprise a fatty acyl group containing at least 8 carbons
  REGION 1..7: note = MISC_FEATURE - cyclic
KRRRRRR

SEQ ID NO: 876 - protein, 10 aa, synthetic construct
  REGION 1..10: note = cyclic CPP
  REGION 1..10: note = MISC_FEATURE - cyclic
  SITE 1: note = MISC_FEATURE - N-acetyl
WWWWKRRRRR

SEQ ID NO: 877 - protein, 4 aa, synthetic construct
  REGION 1..4: note = NLS
RKRH

SEQ ID NO: 878 - protein, 7 aa, synthetic construct
  REGION 1..7: note = SV40 large T antigen NLS
PKKKRKV

SEQ ID NO: 879 - protein, 16 aa, synthetic construct
  REGION 1..16: note = Nucleoplasmin NLS
KRPAATKKAGQAKKKK

SEQ ID NO: 880 - skipped

SEQ ID NO: 881 - protein, 7 aa, synthetic construct
  REGION 1..7: note = NLS variant
  VARIANT 3: note = X can be any amino acid
  VARIANT 3: note = X can be 5 to 20 residues
KRXKKKK

SEQ ID NO: 882 - protein, 16 aa, synthetic construct
  REGION 1..16: note = NLS variant
  VARIANT 3..12: note = X can be any amino acid
KRXXXXXXXXXXKKKK

SEQ ID NO: 883 - protein, 7 aa, synthetic construct
  REGION 1..7: note = NLS variant
  VARIANT 5: note = X can be any amino acid
  VARIANT 5: note = X can be 2 to 10 residues
RKRHXKK

SEQ ID NO: 884 - protein, 8 aa, synthetic construct
  REGION 1..8: note = NLS variant
  VARIANT 5..6: note = X can be any amino acid
RKRHXXKK

SEQ ID NO: 885 - protein, 8 aa, synthetic construct
  REGION 1..8: note = NLS variant
RKRHIIKK

SEQ ID NO: 886 - protein, 9 aa, synthetic construct
  REGION 1..9: note = Oncoprotein c-myc NLS
PAAKRVKLD

SEQ ID NO: 887 - protein, 4 aa, synthetic construct
  REGION 1..4: note = NLS cluster of basic amino acids
KKKK

SEQ ID NO: 888 - protein, 8 aa, synthetic construct
  REGION 1..8: note = NLS variant
PAAKKKLD

SEQ ID NO: 889 - protein, 8 aa, synthetic construct
  REGION 1..8: note = NLS variant
PKKKRKVL

SEQ ID NO: 890 - protein, 5 aa, synthetic construct
  REGION 1..5: note = NLS variant
KKKRK

SEQ ID NO: 891 - protein, 6 aa, synthetic construct
  REGION 1..6: note = NLS variant
KKKRVK

SEQ ID NO: 892 - protein, 7 aa, synthetic construct
  REGION 1..7: note = NLS variant
KKKRKVL

SEQ ID NO: 893 - protein, 7 aa, synthetic construct
  REGION 1..7: note = NLS variant
RKKRKVL

SEQ ID NO: 894 - protein, 5 aa, synthetic construct
  REGION 1..5: note = Linker
IILVI

SEQ ID NO: 895 - protein, 7 aa, synthetic construct
  REGION 1..7: note = Linker
IILVIII

SEQ ID NO: 896 - protein, 4 aa, synthetic construct
  REGION 1..4: note = Linker sequence
GILQ

SEQ ID NO: 897 - protein, 7 aa, synthetic construct
  REGION 1..7: note = Linker sequence
GILQWRK

SEQ ID NO: 898 - protein, 7 aa, synthetic construct
  REGION 1..7: note = linker
WKKKRKI

SEQ ID NO: 899 - protein, 14 aa, synthetic construct
  REGION 1..14: note = APIM Oligopeptidic compound
MDRWLVKRILVATK

SEQ ID NO: 900 - protein, 20 aa, synthetic construct
  REGION 1..20: note = APIM Oligopeptidic compound
MDRWLVKRILKKKRKVATKG

SEQ ID NO: 901 - protein, 34 aa, synthetic construct
  REGION 1..34: note = MDR2
MDRWLVKGAQPKKKRKVLRQIKIWFQNRRMKWKK

SEQ ID NO: 902 - protein, 28 aa, synthetic construct
  REGION 1..28: note = MDR27
MDRWLVKGAWKKKRVKIIRKKRRQRRRK

SEQ ID NO: 903 - protein, 27 aa, synthetic construct
  REGION 1..27: note = MDR26-0
MDRWLVKGAWKKKRKIIRKKRRQRRRG

SEQ ID NO: 904 - protein, 27 aa, synthetic construct
  REGION 1..27: note = MDR26-1
MDRWLVKGAWKKKRKIIRKKRRQRRRK

SEQ ID NO: 905 - protein, 27 aa, synthetic construct
  REGION 1..27: note = MDR26-2
MDRWLVKRIWKKKRKIIRKKRRQRRRK

SEQ ID NO: 906 - protein, 27 aa, synthetic construct
  REGION 1..27: note = MDR26-3
MDRWLVKWWWKKKRKIIRKKRRQRRRK

SEQ ID NO: 907 - protein, 27 aa, synthetic construct
  REGION 1..27: note = MDR26-4
MDRWLVKWWRKRHIIKKRKKRRQRRRK

SEQ ID NO: 908 - protein, 29 aa, synthetic construct
  REGION 1..29: note = MDR26-7
MDRWLVKRIWKKKRKIIRRRRRRRRRRRK

SEQ ID NO: 909 - protein, 33 aa, synthetic construct
  REGION 1..33: note = MDR26-8
MDRWLVKRIWKKKRKIIRQIKIWFQNRRMKWKK

SEQ ID NO: 910 - protein, 26 aa, synthetic construct
  REGION 1..26: note = MDR26-72
MDRWLVKWKKKRKIRRRRRRRRRRRK

SEQ ID NO: 911 - protein, 24 aa, synthetic construct
  REGION 1..24: note = MDR26-32
MDRWLVKWKKKRKIRKKRRQRRRK

SEQ ID NO: 912 - protein, 23 aa, synthetic construct
  REGION 1..23: note = MDR26-42
MDRWLVKWRKRHIRKKRRQRRRK

SEQ ID NO: 913 - protein, 25 aa, synthetic construct
  REGION 1..25: note = MDR24-43
MDRWLVKGAWRKRHIRKKRRQRRRK

SEQ ID NO: 914 - protein, 25 aa, synthetic construct
  REGION 1..25: note = ATX-101
MDRWLVKWKKKRKIRRRRRRRRRRR

SEQ ID NO: 915 - protein, 24 aa, synthetic construct
  REGION 1..24: note = MDR26-72-011
MDRWLVKKKKRKRRRRRRRRRRRK

SEQ ID NO: 916 - protein, 23 aa, synthetic construct
  REGION 1..23: note = MDR26-01
MDRWLVKKKKRKRRRRRRRRRRR

SEQ ID NO: 917 - protein, 35 aa, synthetic construct
  REGION 1..35: note = MDR34
MDRWLVKRIWKKKRKIIRWLVKWWWRKKRRQRRRK

SEQ ID NO: 918 - protein, 23 aa, synthetic construct
  REGION 1..23: note = APIM peptide
RWLVKWKKKRKIRRRRRRRRRRR

SEQ ID NO: 919 - protein, 22 aa, synthetic construct
  REGION 1..22: note = APIM peptide
RWLVKKKKRKRRRRRRRRRRRK

SEQ ID NO: 920 - protein, 21 aa, synthetic construct
  REGION 1..21: note = APIM peptide
RWLVKKKKRKRRRRRRRRRRR